(19) United States
(12) Patent Application Publication
(10) Pub. No.: US 2024/0164355 A1
(43) Pub. Date: May 23, 2024

(54) CYTOPLASMIC INCOMPATIBILITY FACTORS AND METHODS FOR CONTROLLING ANTHROPODS

(71) Applicants: VANDERBILT UNIVERSITY, Nashville, TN (US); YALE UNIVERSITY, New Haven, CT (US)

(72) Inventors: Jason METCALF, Dexter, MI (US); Seth R. BORDENSTEIN, Nashville, TN (US); Daniel LEPAGE, Centertown, MO (US); Sarah BORDENSTEIN, Nashville, TN (US); Mark HOCHSTRASSER, Hamden, CT (US); John F. BECKMANN, New Haven, CT (US); Judith RONAU, Hamden, CT (US)

(21) Appl. No.: 18/494,365

(22) Filed: Oct. 25, 2023

Related U.S. Application Data
(63) Continuation of application No. 16/307,982, filed on Dec. 7, 2018, now Pat. No. 11,832,601.
(60) Provisional application No. 62/347,818, filed on Jun. 9, 2016.

(51) Int. Cl. A01K 67/033; C07K 14/195; C12N 1/20; C12N 9/22; C12N 9/64; C12N 15/87

(52) U.S. Cl. CPC A01K 67/0337; A01K 67/0339; C07K 14/195; C12N 1/205; C12N 9/22; C12N 9/6472; C12N 15/87; A01K 2217/052; A01K 2217/206; A01K 2267/02; C12R 2001/01

(57) ABSTRACT

The disclosure relates to genetically modified bacteria, genetically modified arthropods, and methods for controlling and/or reducing arthropod populations.

### CROSS REFERENCE TO RELATED APPLICATIONS

This application is a national stage application filed under 35 U.S.C. § 371 of PCT/US2017/036693 filed Jun. 9, 2017, which claims the benefit of U.S. Provisional Patent Application Ser. No. 62/347,818 filed Jun. 9, 2016, which are is expressly incorporated herein by reference.

### STATEMENT REGARDING FEDERALLY SPONSORED RESEARCH

This invention was made with government support under 2014-67012-22268 awarded by USDA and under GM 053756, HD086833, GM007347, AI081322, CA068485, DK020593, DK058404, DK059637, and EY008126 awarded by National Institutes of Health and under 151398 and 1456778 awarded by the Nation Science Foundation. The government has certain rights in the invention.

### SEQUENCE LISTING

A Sequence Listing conforming to the rules of WIPO Standard ST. 26 is hereby incorporated by reference. Said Sequence Listing has been filed as an electronic document via PatentCenter encoded as XML in UTF-8 text. The electronic document, created on Jan. 10, 2024, is entitled “10644-022US2”, and is 49,445 bytes in size.

### FIELD

The disclosure relates to genetically modified bacteria, genetically modified arthropods, and methods for controlling and/or reducing arthropod populations.

### BACKGROUND

The genus Wolbachia is an archetype of maternally inherited intracellular bacteria that infect the germline of millions of invertebrate species worldwide and parasitically alter arthropod sex ratios and reproductive strategies to increase the proportion of infected females (the transmitting sex) in the population. The most common of these reproductive manipulations is cytoplasmic incompatibility (CI), typically expressed as embryonic lethality in crosses between infected males and uninfected females. This lethality is completely rescued by females infected with the same or a similar Wolbachia strain.

Cytoplasmic incompatibility (CI) has important applications in disease vector control, and is currently being used in field trials to drive the spread of Dengue-resistant mosquitoes in wild populations through the release of Wolbachia-infected females, and as a biological control mechanism to depress mosquito populations by releasing Wolbachia-infected males incompatible with wild females. Despite more than 40 years of research, the genes by which Wolbachia cause CI remain unknown.

### SUMMARY

Disclosed herein are genetically modified bacteria and genetically modified arthropods useful for controlling and/or reducing populations of arthropods (for example, insects). For the first time, the inventors have identified the genes that encode the cytoplasmic incompatibility factors capable of reproducing the phenomena of cytoplasmic incompatibility. These genes are used to genetically modify bacteria and/or arthropods in order to produce sterile male arthropods and/or to replace a population of target arthropods.

In one aspect, provided herein is a genetically modified arthropod, said arthropod comprising:
a bacterial operon encoding a cytoplasmic incompatibility factor or a variant thereof; and
a promoter operably linked to the bacterial operon;
wherein the expression of the cytoplasmic incompatibility factor in a male arthropod causes a reduction in viable offspring in comparison to a male arthropod lacking the cytoplasmic incompatibility factor.

In another aspect, provided herein is a method for controlling a population of target arthropods, comprising:
providing a bacterial operon encoding a cytoplasmic incompatibility factor or a variant thereof, and a promoter operably linked to the bacterial operon;
transforming a population of male arthropods with the bacterial operon; and
releasing the male arthropods amongst a population of target arthropods, wherein the release of the male arthropods reduces the population of target arthropods.

In one aspect, provided herein is a genetically modified bacterium comprising:
a bacterial operon encoding a cytoplasmic incompatibility factor or a variant thereof; and
a promoter operably linked to the bacterial operon;
wherein the bacterial operon occurs at a non-naturally occurring genomic location in the bacterium.

In another aspect, provided herein is an arthropod infected with a bacterium, wherein the bacterium comprises:
a bacterial operon encoding a cytoplasmic incompatibility factor or a variant thereof; and
a promoter operably linked to the bacterial operon;
wherein the bacterial operon occurs at a non-naturally occurring genomic location in the bacterium.

In an additional aspect, provided herein is a method for controlling a population of target arthropods, comprising:
providing a genetically modified bacterium comprising:
   a bacterial operon encoding a cytoplasmic incompatibility factor or a variant thereof, and
   a promoter operably linked to the bacterial operon;
infecting a population of replacement arthropods with the genetically modified bacterium; and
releasing the replacement arthropods amongst a population of target arthropods, wherein the release of the replacement arthropods reduces the population of target arthropods.

### BRIEF DESCRIPTION OF THE DRAWINGS

The accompanying figures, which are incorporated in and constitute a part of this specification, illustrate several aspects described below.

FIG. 1. Cytoplasmic incompatibility (CI) gene candidate selection and evolution. (a) Venn diagram illustrating unique and shared gene sets from four CI-inducing Wolbachia strains. The number of gene families in common between strains is indicated for each combination. (b) Venn diagram illustrating the number of unique wMel genes matching each criteria combination. Bayesian phylogenies of WD0631 (c) and WD0632 (e) and their homologs are shown based on a 256-aa alignment of WD0631 reciprocal BLASTp hits and a 462-aa alignment of WD0632 reciprocal BLASTp hits. When multiple similar copies of the same operon exist in the same strain, only one copy is shown. Consensus support values are shown at the nodes. Both trees are based on the JTT+G model of evolution and are unrooted. (d) CI patterns correlate with WD0631/WD0632 operon homology. wRi rescues wMel and both share a similar operon (*). The inability of wMel to rescue wRi correlates with an operon type (†) that is present in wRi but absent in wMel. Likewise, bidirectional incompatibility of all other crosses correlates to divergent operons. This diagram was adapted from Bossan et. al⁵¹. (f) Protein architecture of WD0631/WD0632 homologs is conserved for each clade and is classified according to the WD0632-like domain: Type I features Peptidase_C48; Type II lacks an annotated functional domain; and Type III features DUF1703. TM stands for transmembrane domain. For (c) and (e), the WO-prefix indicates a specific phage WO haplotype and the w-prefix refers to a “WO-like island,” a small subset of conserved phage genes, within that specific Wolbachia strain.

FIG. 2. Expression of CI effector candidates decrease as males age. (a-f) Expression of each gene in one-day-old and seven-day-old wMel-infected D. melanogaster testes, as determined by quantitative RT-PCR, is shown relative to groEL. Error bars indicate standard deviation. *=P<0.05, **=P<0.01 by Mann-Whitney U test.

FIG. 3. Dual expression of WD0631 and WD0632 induces CI. Hatch rate assays are shown with either single-gene transgenic (a,c), or dual WD0631/WD0632 transgenic (b,c) D. melanogaster. Infection status is designated with filled-in symbols for a wMel-infected parent or open symbols for an uninfected parent. Transgenic flies are labeled with their transgene to the right of their gender symbol. Unlabeled gender symbols represent wild type flies. Data points are colored according to the type of cross, with blue indicating no Wolbachia infection, red indicating a CI cross with male-only wMel infections, and purple indicating a rescue cross with wMel-infected females. Error bars indicate standard deviation. *=P<0.05, **=P<0.01, ***=P<0.001, ****=P<0.0001 by ANOVA with Kruskal-Wallis test and Dunn's multiple test correction. Statistical comparisons are between all groups for panels a and b; comparisons for panel c are between CI crosses (red) only.

FIG. 4. Dual expression of WD0631 and WD0632 recapitulates cytological defects associated with CI. Representative embryo cytology is shown for (a) unfertilized embryos, (b) normal embryos at one hour of development, (c) normal embryos at two hours of development, and three different mitotic abnormalities: (d) failure of cell division after two to three mitoses, (e) chromatin bridging, and (f) regional mitotic failure. (g) The number of embryos with each cytological phenotype resulting from crosses of dual-expressing WD0631/WD0632 males and uninfected females along with control crosses were counted. Infection status is designated with filled in symbols for a wMel-infected parent or open symbols for an uninfected parent. Transgenic flies are labeled with their transgene to the right of their gender symbol. Unlabeled gender symbols represent wild type flies. Black lines on each graph indicates mean hatch rate for the cross. *=P<0.05, ****=P<0.0001 by two-tailed Fisher's exact test comparing normal (phenotypes b and c) to abnormal (phenotypes a, d, e, and f) for each cross. (h) Quantitation of cytological defects in crosses utilizing WD0508, WD0631, or WD0632 uninfected males.

FIG. 5. CI and the evolution of Wolbachia or phage WO. (a) Diagram showing the effect of parental Wolbachia infection on progeny viability and infection status. CI occurs when males are Wolbachia-infected but females are not. Wolbachia-infected females are able to rescue the viability defect seen in CI crosses and favor spread of the infection through a population of mixed infection status. (b) Bayesian phylogenies based on a 393-aa alignment of WD0723, the wMel ftsZ gene, and its homologs and (c) a 70-aa alignment of WD0640, the phage WO gpW gene, and its homologs. Trees are based on JTT+G and CpRev+I models of evolution, respectively, and are unrooted. Consensus support values are shown at the nodes. (*) indicates that the CI operon is not included in FIG. 1. The WOPip5 operon is truncated while the WOPip2 and second wAlbB operons are highly divergent from WD0632.

FIG. 6. WD0631/WD0632 operon is always associated with prophage WO regions. CI operons are labeled and colored pink. Structural modules are labeled as host adsorption, head or tail. The WD0611-WD0621 label highlights a conserved gene cluster that is often associated with the CI operon. Only one phage haplotype is shown per Wolbachia strain when multiple copies of the same operon type are present.

FIG. 7. Wolbachia CI patterns correlate with WD0631/WD0632 operon similarity and copy number. (a) The % amino acid (aa) identity between homologs for each cif protein correlates with Wolbachia compatibility patterns. The only compatible cross, wMel males×wRi females, features a shared operon between WOMelB and WORiB. All other crosses are greater than 30% divergent and are bidirectionally incompatible. Each “% aa identity” value is based on the region of query coverage in a 1:1 BLASTp analysis. (b) CI strength, protein architecture and operon type are listed for each of the Wolbachia strains shown in FIG. 1d. (*) indicates the proteins are disrupted and not included in comparison analyses.

FIG. 8. Wolbachia titers in wild type and transgenic lines. (a) Relative Wolbachia titers do not decrease with age. DNA copy number of wMel groEL gene is shown normalized to D. melanogaster Rp49 gene copy number in testes at the indicated ages. (b) Absolute Wolbachia titers do not decrease with male age. (c-e) Relative Wolbachia titers are increased in WD0508, WD0631, or WD0632 transgenic lines. This does not occur in the WD0625 transgenic line nor does there appear to be an additive effect. Titers determined by real-time PCR detecting absolute copy number of wMel groEL gene compared to absolute copy number of the D. melanogaster Rp49 gene. Error bars show standard deviation. *=P<0.05, ***=P<0.001, ****=P<0.0001 by ANOVA with Kruskal-Wallis test and Dunn's multiple test correction. Two-tailed Mann-Whitney U test used for (c).

FIG. 9. WD0625 expression does not induce CI. Expression of WD0625 in uninfected males does not affect egg hatch rates (a) or sex ratios (b). Error bars indicate standard deviation. *=P<0.05, ***=P<0.001 by ANOVA with Kruskal-Wallis test and Dunn's multiple test correction.

FIG. 10. Expression of CI effector candidates does not alter sex ratios. (a-c) Graphs correspond to the same crosses as FIG. 3. Infection status is designated with filled in symbols for a wMel-infected parent or open symbols for an uninfected parent. Transgenic flies are labeled with their transgene to the right of their gender symbol. Unlabeled gender symbols represent WT flies. Data points are colored according to the type of cross, with blue indicating no Wolbachia infection, red indicating a CI cross with male-only wMel infections, and purple indicating a rescue cross with wMel-infected females. Error bars indicate standard deviation.

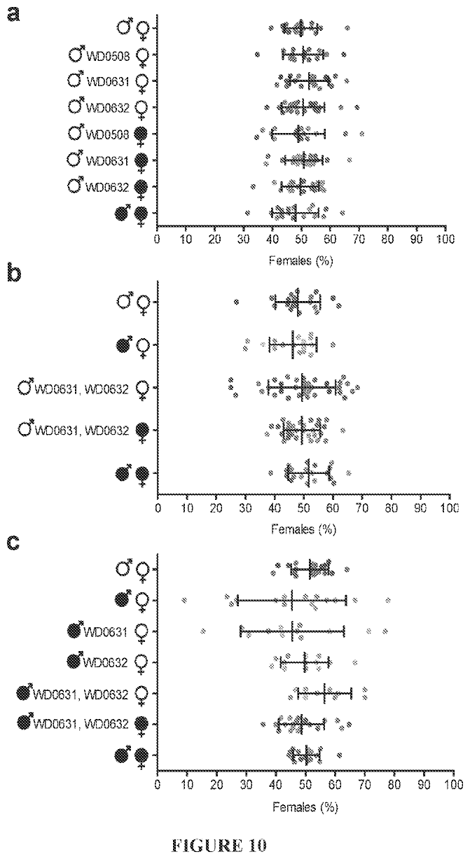

FIG. 11. CI effector candidates are expressed in testes from transgenic flies. WD0508 (a) and WD0625 (b) are expressed in testes as evident by PCR performed against cDNA generated from dissected males utilized in FIG. 3a. (c,d) WD0631 and WD0632 are expressed in the testes from transgenic males inducing high CI, no CI, or rescued CI. Testes were removed from males used in FIG. 3b. (e,f) WD0631 and WD0632 are expressed in ovaries from transgenic females. Ovaries were dissected from females utilized in FIG. 14a.

FIG. 12. Transgenic expression of genes other than WD0631/WD0632 has no effect on hatch rates. (a) The WD0508 transgene does not increase CI in infected males. (b) Addition of WD0625 to WD0632 in wMel-infected males does not lower hatch rates further than WD0632 alone. (c) WD0625/WD0632 dual expression cannot induce CI. Infection status is designated with filled in symbols for a wMel-infected parent or open symbols for an uninfected parent. Transgenic flies are labeled with their transgene to the right of their gender symbol. Unlabeled gender symbols represent wild type flies. Data points are colored according to the type of cross, with blue indicating no Wolbachia infection, red indicating a CI cross with male-only wMel infections, and purple indicating a rescue cross with wMel-infected females. Error bars indicate standard deviation. **=P<0.01, ***=P<0.001, ****=P<0.0001 by ANOVA with Kruskal-Wallis test and Dunn's multiple test correction.

FIG. 13. Transgenic expression of genes other than WD0631/WD0632 has no effect on sex ratios. (a-c) Infection status is designated with filled in symbols for a wMel-infected parent or open symbols for an uninfected parent. Transgenic flies are labeled with their transgene to the right of their gender symbol. Unlabeled gender symbols represent wild type flies. Data points are colored according to the type of cross, with blue indicating no Wolbachia infection, red indicating a CI cross with male-only wMel infections, and purple indicating a rescue cross with wMel-infected females. Error bars indicate standard deviation. Statistics performed by ANOVA with Kruskal-Wallis test and Dunn's multiple test correction.

FIG. 14. WD0631/WD0632 expression in females cannot rescue CI. (a, b) Hatch rates and (c) sex ratios for the indicated crosses are shown. Single expression or dual expression of WD0631 and WD0632 in uninfected females does not reduce embryo hatching or rescue wild-type or induced CI defects. Infection status is designated with shading for a wMel-infected parent or no shading for an uninfected parent. Transgenic flies are labeled with their transgene to the right of their gender symbol. Unlabeled gender symbols represent WT flies. Data points are colored according to the type of cross, with blue indicating no Wolbachia infection, red indicating a CI cross with male-only wMel infections, and purple indicating a rescue cross with wMel-infected females. Error bars indicate standard deviation. Statistics performed by ANOVA with Kruskal-Wallis test and Dunn's multiple test correction.

FIG. 15. Toxin-antidote hypothesis for CI. a. Crossing Wolbachia-infected males (red) with uninfected females (black) yields nonviable embryos due to a sperm-derived toxin. b. Crossing infected males and infected females rescues viability due to antidote in the infected egg. c. Operon from Wolbachia (wPip strain) proposed to induce CI through a toxin-antidote mechanism with CidA (wPa_0282) acting as antidote and CidB (wPa_0283) as toxin. d. Paralogous operon from wPip in which a putative DUF1703 nuclease, CinB (wPa_0295) is the toxin. e. Orthologous cidA-cidB operon from wMel. f. Pulldown assays of operon partners reveal interaction specificity. His6-tagged beta-galactosidase (LacZ) is a negative control.

FIG. 16. Test of the toxin-antidote hypothesis in Saccharomyces cerevisiae. a. Expression of Wolbachia proteins from a galactose-inducible GAL1 promoter on minimal medium lacking uracil and containing galactose or glucose. Controls pYES2 (empty vector) and LacZ (negative control) cause no defects. Both CidB and CinB expression blocks yeast growth at high temperature. Inactivation of the Ulp1-like protease by a C1025A mutation (CidB*) or the putative DUF1703 nuclease by mutation of the D-E-K triad to A-A-A (CinB*) eliminates toxicity. b. Coexpression of “toxins” with different “antidotes” on minimal media lacking uracil and leucine shows growth rescue only with cognate partners. Vector is pRS425.

FIG. 17. CidB is a DUB. a. Anti-HA immunoblot examining DUB reactivity with the HA-tagged suicide inhibitor, Ub-VME. Ub-VME reacts with wild-type and truncated CidB proteins but not the C1025A catalytic mutant (CidB*). TsUCH37 is a positive control.⁴⁰ CidA at 100-fold molar excess does not inhibit Ub-VMe reactivity. (#) is putative UbVME2 b. Anti-ubiquitin (Ub) immunoblot of K48- and K63-linked ubiquitin chains showing cleavage by CidB. Usp2 is a positive control.⁴¹ Enzyme and substrate were 50 nM and 500 nM, respectively, and reactions were at 37° C. for 1 h. c. CidB has a ˜4-fold preference for K63-Ub dimers compared to K48-linked dimers.

FIG. 18. Induction of CI by transgenic cidA-cidB. a. D. melanogaster males carrying transgenic cidA-cidB are sterile when mated to WT (^{w}CS) females (n=30). Males with transgenic cidA-cidB* harboring a CidB active-site mutation C1025A (operon*) are fully fertile (n=27). Females with the transgenic operon are fertile. CidA by itself has no effect on fertility, while no strain singly transgenic for cidB could be isolated. EGFP is a negative control. Error bars are standard deviations. b. CI-like defects in the male pronucleus initially appear in late prophase, during the first division of the apposed female and male pronuclei, and accrue through mitosis.

FIG. 19. Homology analysis of putative toxins and antidotes. a. Percent similarity using BLAST of the wPip CidA antidote and CidB toxin as queries against other antidotes and toxins. DUB-based operons from wPip and wMel Wolbachia strains share higher similarity and are more closely related than they are to the nuclease-type operon from wPip. b. Secondary structure predictions by Psipred shows an underlying conserved architecture between CidB and CinB. Both toxins show a homologous αβββαβ fold characteristic of the predicted DUF1703 nuclease. However, only CinB maintains a complete catalytic D-E-K triad (black boxes), and in CidB the αβββαβ fold is interrupted by an insertion. In CidB this nuclease-like fold is N-terminal to the additional DUB catalytic domain (FIG. 15c, dotted lines). This evidence is consistent with a duplication and divergence from a common CinB-like ancestral operon.

FIG. 20. His6 pulldowns of recombinant CidA shows C-terminal cleavage by E. coli Lon protease but not Wolbachia Lon protease. a. Coomassie SDS-PAGE analysis of recombinant CidA protein expression. Lane 1, pBadB vector; 2, N-terminally tagged His6-CidA (#AX1); 3, doubly tagged His6-CidA-His6 (#AW1); 4, C-terminally tagged CidA-His6 (#Y10); 5, a codon-optimized variant of N-terminally tagged His6-CidA (#AS1); 6, N-terminally tagged His6-CidA in BL21-AI cells with a deletion of the lon protease gene (#N15); and 7, N-terminally tagged His6-CidA coexpressed with His6-tagged Wolbachia Lon Protease (#BN5). Switching expression of recombinant proteins from TOP1OF′ cells to BL21-AI cells (which lack Lon protease) eliminated the doublet (Lane 6). Because Lon often regulates toxin-antidote systems, tested Wolbachia's own Lon protease was tested, but it did not cleave CidA (lane 7). Subsequent expression of CidA and other proteins was always performed in BL21-AI or Rosetta cells lacking Lon. b. Anti-His6 immunoblot corroborates the Coomassie staining patterns.

FIG. 21. Interaction of toxin-antidote proteins. a. His6 pulldowns reveal binding interactions of operon partners. Lanes 1, 2 and 3 are His6-CidA, His6-CidB, and His6-CidA-CidB (full operon), respectively. b. Western blot analysis verifying that the co-pelleted species is CidB. CidB is C-terminally FLAG-tagged in lanes 4 and 6. c. His6 pulldowns show interactions of CinA to CinB. Lanes 1, 2 and 3 are His6-CinA, His6-CinB, and His6-CinA-CinB (full operon), respectively. d. Western blot analysis verifying that the co-pelleted species is CinB. FLAG tags are analogous to panel b.

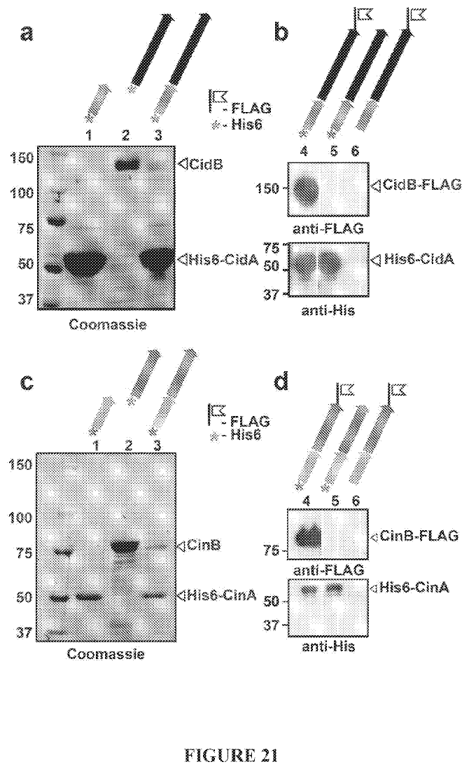

FIG. 22. Yeast heterologous protein expression controls. A. Western immunoblotting of FLAG-tagged CidB and CinB proteins expressed from the yeast 2-micron plasmid pYES2 (GAL1 promoter). Closed green circles indicate 2% galactose (induced) in the growth medium, open green circles, 2% glucose (repressed). CinB and the catalytically inactivated CidB* (C1025A) are expressed at similar levels. The catalytically inactivated mutant (D614A; E634A; K636A), CinB*, does appear to be expressed at lower levels, and this could account at least in part for decreased toxicity. However, when CinB was expressed from a low-copy (CEN) expression plasmid (FIG. 22b), the protein level is lower than CinB* expression from the high-copy vector, yet toxicity was still observed for CinB. This suggests that enzyme inactivation, rather than reduced protein amount, caused the reduced toxicity of the inactive nuclease. b. Western blotting of FLAG-tagged CidB and CinB proteins expressed from the low-copy pRS416 (GAL1) plasmid. Genes for the co-expressed putative antidotes were cloned into the high-copy 2-micron pRS425 (GAL1) vector. PGK is a loading control.

FIG. 23. Cleavage of ubiquitin dimers. a. Full length CidB cleaves all forms of lysine-linked (isopeptide-linked) diubiquitin, albeit with variable efficiency, but is inactive on linear Met1-linked diubiquitin. Digests of diubiquitin were performed overnight at 37° C. with enzyme and substrate both at 1 μM concentration. Similar results were observed with shorter digests of 1 or 4 h. b. Representative kinetic assay of diubiquitin cleavage. Lanes 1-3 are ubiquitin standards of 6, 20, and 40 μM, respectively. In lanes 4-8, 400 nM CidB (762-1143) was incubated with Lys48-linked diubiquitin ranging in concentration from 20 μM (lane 4) to 120 μM (lane 8). All Lys48-linked diubiquitin reactions were carried out at room temperature for 15 min. The amount of ubiquitin produced from each reaction was quantified by densitometry using ImageJ software. c. Total cellular ubiquitylation as measured by anti-ubiquitin immunoblotting in yeast extracts. Induction of GAL1-driven CidB expression did not change the pattern of ubiquitin conjugates when compared to cells with induced CidA or CidB* (negative controls). These results suggest CidB activity is likely limited to a small number of cellular substrates rather than affecting gross protein ubiquitylation. The same sample was run on two separate gradient gels of 4-10% and 10-15% gels to create maximal separation of high and low molecular weight ubiquitylated species. Induction temperatures of 30° ° C. and 37° C. in the presence of galactose for 4 h were utilized because toxicity is most apparent at 37° C.

FIG. 24. Cleavage of Ub-AMC and Nedd8-AMC by wPip and wMel CidB enzymes. a. Progress curves of AMC release from Ub-AMC and Nedd8-AMC catalyzed by CidB^{wPip} and CidB^{wMel} are depicted. Enzyme (5 nM) was mixed with 400 nM of substrate, and the reactions proceeded at 30° C.; the enzymes share a similar preference for ubiquitin over the UBL Nedd8. The activity of CidB^{wMel} is comparable to CidB^{wPip}. b. CidB^{wPip} catalytic efficiency for hydrolyzing Ub-AMC is 11-fold greater than for Nedd8-AMC. c. As the kinetics from FIG. 24b exhibited a linear response over the substrate concentration range tested, which is typical of other DUBs toward the Ub-AMC substrate, the k_{cat}/K_{M} values were determined by fitting the data to the equation: v/[E]=k_{cat}/K_{M}[S]. d. Steady-state kinetic parameters for CidB^{wPip} cleavage of Lys63- and Lys48-linked ubiquitin dimers indicate a modest preference for Lys63-linked diubiquitin. This suggests that the physiological targets of CidB^{wPip} might bear Lys63-polyubiquitin linkages and are less likely to be targets of Lys48-polyubiuqitin-based proteasomal degradation.

FIG. 25. Creation of transgenic D. melanogaster strains. a. pUASp-attB vector.^{32,33} UAS is the GAL4 upstream activating element; P is the P-element basal germline promoter; and MCS is the multi-cloning site. K10 has 3′UTR sequences from the K10 terminator, and attB is the ΦC31 integrase recombination site. b. Five transgene injection constructs were created by heterologous gene insertion into pUASp-attB: four cidA-cidB-derived constructs and an EGFPC1 negative control. T2A is a viral peptide sequence that causes translation of two separate polypeptides from the fused ORFs by ribosome skipping, mimicking the bicistronic bacterial operon; no efficient IRES system has been described for D. melanogaster. No transgenic lines expressing CidB^{wPip} alone could be established after 3 trials totaling 600 embryo microinjections, whereas all other constructs readily recombined into the Drosophila chromosome-3 attP site. c. Transgenic fly lines were created and screened for proper attB/attP recombination by PCR. “AttL” is a PCR product indicating correct recombination. Rps3 is a positive PCR control. The ^{w}CS and YW lanes are negative controls using genomic DNA from these two untransformed fly strains. Multiple transgenic fly lines were created for each construct. A total of four sterile “Operon” (cidA-cidB) lines were created in two different fly backgrounds bearing independent attP insertion sites (#9744 and #9750). Four independent lines with the catalytically inactive DUB (Operon*) were isolated in the #9744 background. All replicate lines showed the same phenotypes. d. Verification that lines used in transgenic crosses (#9744, 9750, and ^{w}CS) were uninfected with native Wolbachia strains. CidA^{wMel} (WD_0631) is the antidote protein from the wMel Wolbachia strain. e. Reverse transcriptase-PCR analysis confirming transcription of the transgenic operons from the basal P-element promoter despite the absence of a Gal4 driver. DNA is a positive PCR control to show correct band size. RNA samples from pooled adult males were assayed with reverse transcriptase “(+) RT”-PCR to verify the presence of transcript; omission of reverse transcriptase, “(−) RT,” served as a negative control for DNA contamination. The cDNAs were amplified with primers specific for CidB^{wPip}. Analysis of the fly Rps3 transcript was a positive control for RNA quality. As in panel c, the Operon and Operon* (cidA-cidB wPip) fly lines express active CidB and catalytically inactive CidB* (C1025A), respectively.

FIG. 26. Quantification of transgenic cidA-cidB embryo cytology. a. Developmental progress of transgenic (“CI”) embryos. After 24 h, embryos were classified into three categories. Early, pre-blastoderm formation; Mid, blastoderm until segmentation stages; and Late, segmented stages. Quantification is based on three samples of approximately 200 embryos each. 60% of CI embryos arrested development in the early stage compared to 12% from the wild-type (WT) control. Significant p values <0.005 are indicated by (*). b. Quantification of transgenic cidA-cidB (CI) embryos' mitotic defects including uncondensed paternal chromosomes, delayed segregation of paternal chromosomes, or chromosomal bridging during the first cell cycle. 88% of CI embryos fixed and characterized during this stage exhibited these CI-like defects as compared to 3% in the WT control. Sample sizes of observed transgenic and WT embryos were 63 and 29, respectively. c. Examples of late-stage embryos; transgenic embryos that develop to the late stage show significant deformations of segmentation patterns including pinching, gaps in segmentation, and asymmetry. d. Of the 20% of transgenic CI embryos that develop to the late stage, 69% showed deformations and abnormal segmentation.

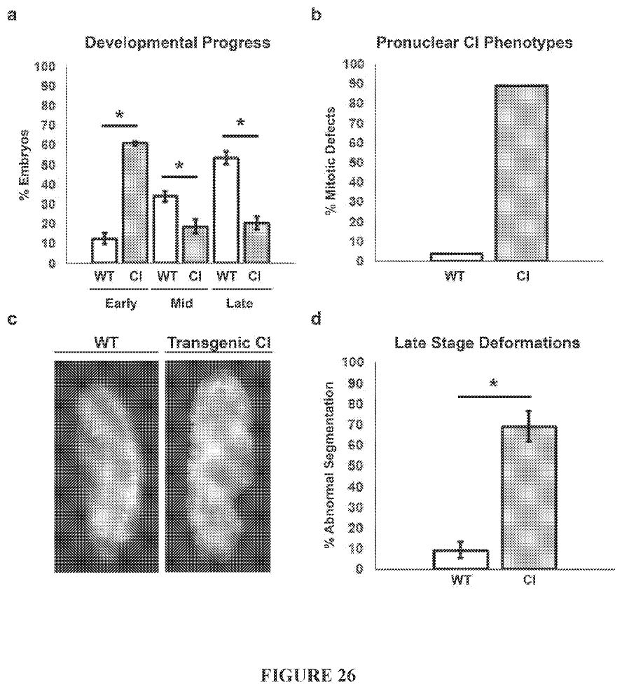

### DETAILED DESCRIPTION

Disclosed herein are genetically modified bacteria and genetically modified arthropods useful for controlling and/or reducing populations of arthropods (for example, insects). For the first time, the inventors have identified the genes that encode the cytoplasmic incompatibility factors capable of reproducing the phenomena of cytoplasmic incompatibility. These genes are used to genetically modify bacteria and/or arthropods in order to produce sterile male arthropods and/or to replace a population of target arthropods.

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this invention belongs. The following definitions are provided for the full understanding of terms used in this specification.

### Terminology

As used herein, the article “a,” “an,” and “the” means “at least one,” unless the context in which the article is used clearly indicates otherwise.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett., 22:1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. In some embodiments, the polynucleotide is composed of nucleotide monomers of generally greater than 100 nucleotides in length and up to about 8,000 or more nucleotides in length.

The term “polypeptide” refers to a compound made up of a single chain of D- or L-amino acids or a mixture of D- and L-amino acids joined by peptide bonds.

The term “complementary” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The term “anneal” refers to the process by which a single-stranded nucleic acid sequence pairs by hydrogen bonds to a complementary sequence, forming a double-stranded nucleic acid sequence, including the reformation (renaturation) of complementary strands that were separated by heat (thermally denatured).

The term “melting” refers to the denaturation of a double-stranded nucleic acid sequence due to high temperatures, resulting in the separation of the double strand into two single strands by breaking the hydrogen bonds between the strands.

The term “target” refers to a molecule that has an affinity for a given probe. Targets may be naturally-occurring or man-made molecules. Also, they can be employed in their unaltered state or as aggregates with other species.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein.

A polynucleotide sequence is “heterologous” to a second polynucleotide sequence if it originates from a foreign species, or, if from the same species, is modified by human action from its original form. For example, a promoter operably linked to a heterologous coding sequence refers to a coding sequence from a species different from that from which the promoter was derived, or, if from the same species, a coding sequence which is different from naturally occurring allelic variants.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above. The term “expression cassette” refers to a nucleic acid construct, which when introduced into a host cell, results in transcription and/or translation of a RNA or polypeptide, respectively. In embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning—A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In some embodiments, an expression cassette comprising a terminator (or termination sequence) operably linked to a second nucleic acid (e.g. polynucleotide) may include a terminator that is heterologous to the second nucleic acid (e.g. polynucleotide) as the result of human manipulation. In some embodiments, the expression cassette comprises a promoter operably linked to a second nucleic acid (e.g. polynucleotide) and a terminator operably linked to the second nucleic acid (e.g. polynucleotide) as the result of human manipulation. In some embodiments, the expression cassette comprises an endogenous promoter. In some embodiments, the expression cassette comprises an endogenous terminator. In some embodiments, the expression cassette comprises a synthetic (or non-natural) promoter. In some embodiments, the expression cassette comprises a synthetic (or non-natural) terminator.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The phrase “codon optimized” as it refers to genes or coding regions of nucleic acid molecules for the transformation of various hosts, refers to the alteration of codons in the gene or coding regions of polynucleic acid molecules to reflect the typical codon usage of a selected organism without altering the polypeptide encoded by the DNA. Such optimization includes replacing at least one, or more than one, or a significant number, of codons with one or more codons that are more frequently used in the genes of that selected organism. For example, the sequence of a heterologous gene expressed in Wolbachia may be “codon optimized” to optimize gene expression based on the preferred codon usage in Wolbachia.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

“Transformation” refers to the transfer of a nucleic acid molecule into a host organism (e.g. Wolbachia cell). In embodiments, the nucleic acid molecule may be a plasmid that replicates autonomously or it may integrate into the genome of the host organism. Host organisms containing the transformed nucleic acid molecule may be referred to as “transgenic” or “recombinant” or “transformed” organisms. A “genetically modified” organism (e.g. genetically modified arthropod) is an organism that includes a nucleic acid that has been modified by human intervention. Examples of a nucleic acid that has been modified by human intervention include, but are not limited to, insertions, deletions, mutations, expression nucleic acid constructs (e.g. over-expression or expression from a non-natural promoter or control sequence or an operably linked promoter and gene nucleic acid distinct from a naturally occurring promoter and gene nucleic acid in an organism), extra-chromosomal nucleic acids, and genomically contained modified nucleic acids.

The term “bacterial operon” as used herein refers to a gene or multiple genes transcribed from a single promoter which leads to the production of a single transcript in which one or more coding regions are linked.

The term “cytoplasmic incompatibility (CI) factor” or “cytoplasmic incompatibility (CI) gene” refers to the genes or the factors encoded by the genes from bacteria which provide a function that is required and/or beneficial to produce the natural genetic drive mechanism of cytoplasmic incompatibility (CI) used by various, unrelated bacterial infections (e.g., Wolbachia and Cardinium endosymbionts). “Cytoplasmic incompatibility (CI) factors” can include those factors that induce the CI and can also include those rescue factors that counteract the CI. In some embodiments, a single bacterial operon may encode multiple cytoplasmic incompatibility (CI) factors. In some embodiments, a single bacterial operon may encode a factor that induces the CI and can also encode a factor that can counteract the CI (for example, a rescue factor).

The term “variant” or “derivative” as used herein refers to an amino acid sequence derived from the amino acid sequence of the parent protein having one or more amino acid substitutions, insertions, and/or deletions. For example, a “cytoplasmic incompatibility (CI) factor variant” includes cytoplasmic incompatibility (CI) factor that may have a number of amino acid changes. In some embodiments, the variants may be greater than about 20%, greater than about 30%, greater than about 40%, greater than about 50%, greater than about 60%, greater than about 70%, greater than about 80%, greater than about 90%, or greater than about 95%, identical to the parent nucleic acid sequence or amino acid sequence.

Wolbachia and Cytoplasmic Incompatability

Wolbachia pipientis is an obligate, intracellular a-proteobacteria and a member of the Rickettsiales family. These gram-negative bacteria are not culturable outside of host cells and, as a result, knowledge on Wolbachia symbiosis has only surged in the last two decades owing to readily available molecular techniques. Once considered an obscure bacterium in a few insect species, the most recent meta-analysis estimates that ˜40% of all arthropod species are infected with Wolbachia as well as 47% of the Onchocercidae family of filarial nematodes.

The genus Wolbachia is an archetype of maternally inherited intracellular bacteria that infect the germline of millions of invertebrate species worldwide and parasitically alter arthropod sex ratios and reproductive strategies to increase the proportion of infected females (the transmitting sex) in the population. The most common of these reproductive manipulations is cytoplasmic incompatibility (CI), typically expressed as embryonic lethality in crosses between infected males and uninfected females. This lethality is completely rescued by females infected with the same or a similar Wolbachia strain. Despite more than 40 years of research, the genes by which Wolbachia cause CI remained unknown until the inventors isolated the genes encoding cytoplasmic instability factors from several strains of Wolbachia.

For the first time, the inventors have determined the genes encoding the cytoplasmic incompatibility factors capable of reproducing the phenomena of cytoplasmic incompatibility. These genes are used to genetically modify bacteria and/or arthropods in order to produce sterile male arthropods and/or to replace a population of target arthropods (for example, replacement of a target population with arthropods that are less susceptible to infectious agents or have a reduced capacity to transmit an infectious agent (for example, dengue virus or Zika virus)).

In one embodiment, the genes encoding the cytoplasmic incompatibility factors are from wMel, for example, WD0631 (SEQ ID NO: 1) and/or WD0632 (SEQ ID NO:3).

In one embodiment, the bacterial operon encodes the cytoplasmic incompatibility factor WD0631 (SEQ ID NO:2). In one embodiment, the bacterial operon encodes the cytoplasmic incompatibility factor WD0632 (SEQ ID NO:4). In one embodiment, the bacterial operon encodes the cytoplasmic incompatibility factors WD0631 and WD0632.

In one embodiment, the bacterial operon encodes a cytoplasmic incompatibility factor of the amino acid sequence SEQ ID NO:2. In one embodiment, the bacterial operon encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:2. In one embodiment, the bacterial operon encodes a cytoplasmic incompatibility factor of the amino acid sequence SEQ ID NO:4. In one embodiment, the bacterial operon encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:4.

In one embodiment. the genes encoding the cytoplasmic incompatibility factors are from Wolbachia pipientis, for example, CidA^{wPip} (wPa_0282; SEQ ID NO:5), CidB^{wPip} (wPa_0283; SEQ ID NO:7), CinA^{wPip} (wPa_0294; SEQ ID NO:17), and/or CinB^{wPip} (wPa_0295; SEQ ID NO:19).

In one embodiment, the bacterial operon encodes the cytoplasmic incompatibility factor CidA^{wPip} (wPa_0282; SEQ ID NO:6). In one embodiment, the bacterial operon encodes the cytoplasmic incompatibility factor CidB^{wPip} (wPa_0283; SEQ ID NO:8). In one embodiment, the bacterial operon encodes the cytoplasmic incompatibility factors CidA^{wPip} (wPa_0282) and CidB^{wPip} (wPa_0283). In one embodiment, the bacterial operon encodes the cytoplasmic incompatibility factor CinA^{wPip} (wPa_0294; SEQ ID NO:18). In one embodiment, the bacterial operon encodes the cytoplasmic incompatibility factor CinB^{wPip} (wPa_0295; SEQ ID NO:20). In one embodiment, the bacterial operon encodes the cytoplasmic incompatibility factors CinA^{wPip} (wPa_0294) and CinB^{wPip} (wPa_0295).

In one embodiment, the bacterial operon encodes a cytoplasmic incompatibility factor of the amino acid sequence SEQ ID NO:6. In one embodiment, the bacterial operon encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:6. In one embodiment, the bacterial operon encodes a cytoplasmic incompatibility factor of the amino acid sequence SEQ ID NO:8. In one embodiment, the bacterial operon encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:8.

In one embodiment, the bacterial operon encodes a cytoplasmic incompatibility factor of the amino acid sequence SEQ ID NO:18. In one embodiment, the bacterial operon encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:18. In one embodiment, the bacterial operon encodes a cytoplasmic incompatibility factor of the amino acid sequence SEQ ID NO:20. In one embodiment, the bacterial operon encodes a cytoplasmic incompatibility factor at least 60% identical (for example, at least 60%, at least 65%, at least 70%, at least 75%, at least 80%, at least 85%, at least 90%, at least 95%, at least 99%) to the amino acid sequence SEQ ID NO:20.

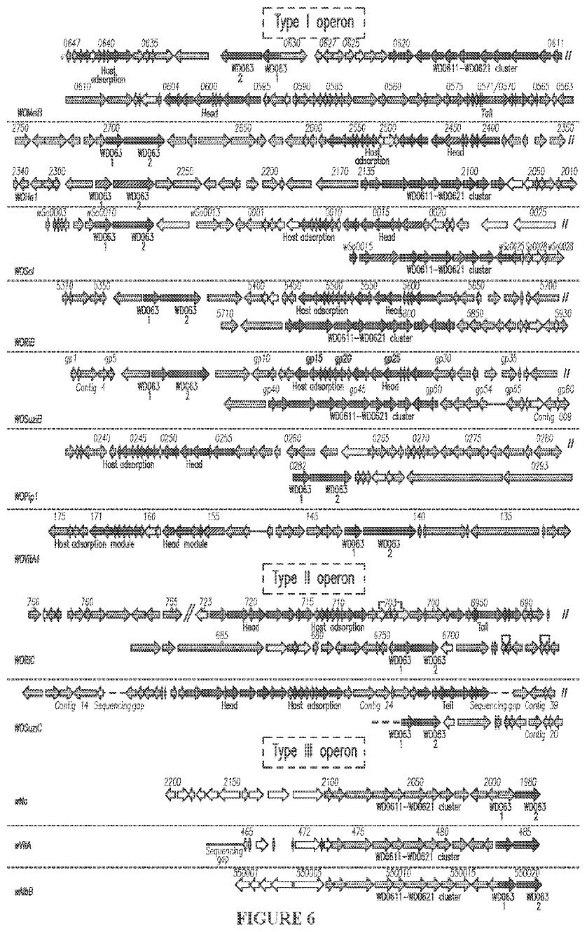

Additional examples of cytoplasmic incompatibility factors include homologues of WD0631 and WD0632 in additional Wolbachia strains including, but not limited to, WOMelB, WOHa1, WOSol, WORIB, WOSuziB, WOPip1, WOVitA4, WORIC, WOSuziC, wNo, wVitA, and/or wAlbB (See FIG. 6).

In some embodiments, a bacterial operon or a gene encoding a cytoplasmic incompatibility factor may be codon optimized, without changing the resulting polypeptide sequence. In some embodiments, the codon optimization includes replacing at least one, or more than one, or a significant number, of codons with one or more codons that are more frequently used in the genes of that selected arthropod. For example, the sequence of a bacterial operon or a gene encoding a cytoplasmic incompatibility expressed in, for example, an Aedes mosquito, may be “codon optimized” to optimize gene expression based on the preferred codon usage in Aedes.

Non-limiting examples of Type I bacterial operons, Type II bacterial operons, Type III bacterial operons, and additional homologues are listed in Table 1, Table 2, Table 3, and Table 4, respectively. Type I CI bacterial operons are Ulp1 type operons and were queried using WP0283 in Table 1. Type II CI bacterial operons are “No CTD” type operons and were queried using wRi_006170 in Table 2. Type III CI bacterial operons are DUF1703 type operons and were queried using wNo_01980 in Table 3. Additional homologues in Table 4 were queried by the gene listed in the table. Additional chromosomal Ulp1 containing loci are listed in Table 5.

TABLE 1
Non-limiting Examples of Type I CI Bacterial Operons

| | cidB Locus | | Wolbachia | | Query | |
|---|---|---|---|---|---|---|
| cidA | tag | cidB Accession | strain | Insect Host | coverage | Identity |
| WP0282 | WP0283 | WP_012481788.1 | wPip | Culex pipiens | 100% | 100% |
| | | | | quinquefasciatus | | |
| | | | | Pel | | |
| | WP1291/ | WP_012482028.1/ | wPip | Culex pipiens | 30% | 99% |
| | WP1292 | WP_012481788.1 | | quinquefasciatus | | |
| | | | | Pel | | |
| (incomplete contig) | C1A_1260 | EEB55318.1 | wPipJHB | Culex | 51% | 100% |
| | | | | quinquefasciatus | | |
| | | | | JHB | | |
| C1A_1298 | C1A_1299 | EEB55179.1 | wPipJHB | Culex | 61% | 97% |
| | | | | quinquefasciatus | | |
| | | | | JHB | | |
| C1A_1302 | C1A_1300/ | EEB55246.1 | wPipJHB | Culex | 28/40% | 96/98% |
| | C1A_1301 | | | quinquefasciatus | | |
| | | | | JHB | | |
| C1A_1344 | C1A_1343 | EEB55169 & | wPipJHB | Culex | 23% | 99% |
| | | WP_007301817.1 | | quinquefasciatus | | |
| | | | | JHB | | |
| | C1A_1356 | EEB55171.1 | wPipJHB | Culex | 14% | 100% |
| | | | | quinquefasciatus | | |
| | | | | JHB | | |
| (gap in contig) | C1A_RS07400 | WP_050751958.1 | wPipJHB | Culex | 21% | 55% |
| | | | | quinquefasciatus | | |
| | | | | JHB | | |
| WPM_01119 | WPM_01122 | CQD10800 & | wPipMol | Culex pipiens | 30% | 99% |
| | | WP_012481788.1 | | Molestus | | |
| WD0631 | WD0632 | WP_010962722.1 | wMel | Drosophila | 99% | 76% |
| | | | | melanogaster | | |
| WMELPOP_03523 | WMELPOP_ | ERN55637.1 & | wMelPopcorn | Drosophila | 99% | 76% |
| | 03528 | WP_038228284.1 | | melanogaster | | |
| wRec0566 | wRec0567 | JQAM01000018.1* | wRec | Drosophila | | |
| | | | | recens | | |
| wHa_02700 | wHa_02690 | YP_007888921.1 & | wHa | Drosophila | 100% | 67% |
| | | WP_015588932.1 | | simulans | | |
| wHa_02280 | wHa_02270 | WP_041573504.1 | wHa | Drosophila | 35% | 74% |
| | | | | simulans | | |
| wRi_05370 | wRi_p05380 | WP_041582611.1 | wRi | Drosophila | 16% | 82% |
| | | | | simulans | | |
| wRi_010030 | wRi_p10040 | WP_041582611.1 | wRi | Drosophila | 16% | 82% |
| | | | | simulans | | |
| WwSim0304 | WwSim0305/ | EAL59981.1/ | wSim | Drosophila | 16%/20% | 81%/66% |
| | WwSim0306 | EAL59982.1 | | simulans | | |
| (incomplete contig) | WwSim0307 | EAL59913.1 | wSim | Drosophila | 46% | 82% |
| | | | | simulans | | |
| (contig 004 - | | WP_044471243.1 | wSuzi | Drosophila | 99% | 76% |
| CAOU02000034.1*) | | | | suzukii | | |
| (contig 005 - | | WP_044471243.1 | wSuzi | Drosophila | 99% | 76% |
| CAOU02000021.1*) | | | | suzukii | | |
| (incomplete contig) | wAna0112 | EAL58062.1 | wAna | Drosophila | 16% | 78% |
| | | | | ananassae | | |
| gwv_142 | gwv_141 | PRJDB1504* | wVitA | Nasonia | | |
| | | | | vitripennis | | |
| wSo0010 | wSo0010 | AGK87078.1 | wSol | Ceratosolen | 100% | 67% |
| | | | | solmsi | | |
| wBol1_RS02195 | wBol1_ | WP_019236548 | wBol1-b | Hypolimnas | 100% | 93% |
| | RS02190 | | | bolina | | |
| TV41_RS03095 | TV41_ | WP_064085577.1 | wDacB | Dactylopius | 100% | 93% |
| | RS03090 | | | coccus | | |
| (incomplete contig) | wGmm_0957/ | KDB19421.1/ | wGmm | Glossina | 22%/39% | 74%/44% |
| | wGmm_0598 | KDB19420.1 | | morsitans | | |
| | | | | morsitans | | |

TABLE 2
Non-limiting Examples of Type II CI Bacterial Operons

| | | | Wolbachia | Insect | Query | |
|---|---|---|---|---|---|---|
| cixA | cixB Locus tag | cixB Accession | strain | Host | coverage | Identity |
| wRi_006720 | wRi_006710 | YP_002727222.1 & | wRi | Drosophila | 100% | 100% |
| | | WP_012673227.1 | | simulans | | |
| (incomplete contig) | WwSim0727 | EAL59553 | wSim | Drosophila | 34% | 100% |
| | | | | simulans | | |
| (incomplete contig) | WwSim0783 | EAL59591.1 | wSim | Drosophila | 28% | 100% |
| | | | | sinulans | | |
| (contig 20 - | (contig 20 - | WP_044471251.1 | wSuzi | Drosophila | 100% | 99% |
| CAOU02000023.1*) | CAOU02000023.1*) | | | suzukii | | |
| (incomplete contig) | wAna_0864/ | WP_007549286.1/ | wAna | Drosophila | 25%/74% | 100/100% |
| | wAna_0865 | WP_007549287.1 | | ananassae | | |

TABLE 3
Non-limiting Examples of Type III CI Bacterial Operons

| | | | Wolbachia | | Query | |
|---|---|---|---|---|---|---|
| cinA | cinB Locus tag | cinB Accession | strain | Insect Host | coverage | Identity |
| wNo_01990 | wNo_01980 | YP_007885511.1 & | wNo | Drosophila | 100% | 100% |
| | | WP_015587805.1 | | simulans | | |
| WP0294 | WP0295 | YP_001975096.1 & | wPip | Culex pipiens | 98% | 31% |
| | | WP_007302979.1 & | | quinquefasciatus | | |
| | | CAQ54403.1 | | Pel | | |
| C1A_1254 | C1A_1253 | EEB55311.1 & | wPipJHB | Culex | 98% | 31% |
| | | WP_007302979.1 | | quinquefasciatus | | |
| | | | | JHB | | |
| | WPM_01094 | CQD10626 & | wPipMol | Culex pipiens | 98% | 31% |
| | | WP_007302979.1 | | Molestus | | |
| wAlbB_120005 | wAlbB_120006 | CCE77108.1 & | wAlbB | Aedes | 98% | 31% |
| | | WP_006012795.1 | | albopictus | | |
| wAlbB_550019 | wAlbB_550020 | CCE77513.1 & | wAlbB | Aedes | 100% | 99% |
| | | WP_006014164.1 | | albopictus | | |
| wBol1_RS01705 | wBol1_RS01710 | WP_019236480 | wBol1-b | Hypolimnas | 98% | 31% |
| | (or WBOL1_01725) | | | bolina | | |
| gwv_484 | gwv_485 | PRJDB1504* | wVitA | Nasonia | | |
| | | | | vitripennis | | |
| wUni_598 | wUni_599 | * | wUni | Muscidifurax | | |
| | | | | uniraptor | | |

TABLE 4
Non-limiting Examples of Additional CI Bacterial Operons

| | | WP0283-like | | Bacterial | | Query | |
|---|---|---|---|---|---|---|---|
| WP0282-like | WP0283-Iike | Accession | Description | strain | Query | coverage | Identity |
| RGRA_ | RGRA_ | WP_024547315.1 | polymorphic, | Candidatus | wwNo_01980 | 41% | 28% |
| RS0104630 | RS0104635 | | DUF1703 | Rickettsia | | | |
| | | | | gravesii | | | |
| JS61_08060 | JS61_08070 | WP_024547315.1 | polymorphic | Rickettsia | wNo_01980 | 67% | 26% |
| | | | | felis | | | |
| | CHV_RS01335 | WP_034576592.1 | | Cardinium | wNo_01980 | 41% | 27% |
| | | | | endosymbiont | | | |
| | | | | of Bemisia | | | |
| | | | | tabaci | | | |
| WSTR_ | WSTR_05300 | WP_063631194.1 | polymorphic | Wolbachia | wNo_01980 | 95% | 24% |
| 05295 | | | | of Laodelphax | | | |
| | | | | striatella | | | |
| TV41_ | TV41_ | WP_064085536.1 | polymorphic, | Wolbachia | wNo_01980 | 87% | 25% |
| RS02770 | RS02775 | | Ulp1 | of Dactylopius | | | |
| | | | | coccus | | | |
| | AlI_06850 | ABV79684.1 | polymorphic, | Rickettsia | wPa_0283 | | |
| | | | Ulp1 | bellii | (894-1177) | | |
| | RPR_06665 | ACR47821.1 | polymorphic, | Rickettsia | wPa_0283 | | |
| | | | Ulp1 | peacockii | (894-1177) | | |

TABLE 5
Non-limiting Examples of Chromosomal Ulp1 Containing Loci

| Ulp1 containing locus tag | Accession | Wolbachia strain | Insect Host |
|---|---|---|---|
| WP1291 | YP_001976023.1 | wPip | Culex pipiens |
| | | | quinquefasciatus Pel |
| WPM_001053c | CDH88846.1 | wPipMol | Culex pipiens Molestus |
| wBm_0463 | YP_198293.1 | wBm | Brugia malayi |
| WRi_000250 | YP_002726686.1 | wRi | D. simulans |
| wHa_00230 | YP_007888701.1 | wHa | D. simulans |
| WD0027 | WP_010962314.1 | wMel | D. melanogaster |
| WMELPOP_00953 | ERN56121.1 | wMelPopcorn | D. melanogaster |
| wPAU_0024 | CDR78424.1 | wAu | D. Simulans |
| wNo_02560 | WP_015587858.1 | wNo | D. simulans |

Methods of Controlling Arthropod Populations: Sterile Insect Technique (SIT)

The concept of the sterile insect technique (SIT) was first discovered by Knipling in 1955 (Knipling, E. F. J Econ Entomol 48, 459-462 (1955)). SIT is the use of sterile males to suppress populations of insects. SIT works by periodic controlled releases of vast numbers of sterile male insects into wild populations. In principle, these sterile males outnumber and outcompete wild males for matings with wild females. If a female mates with a sterile male she will lay eggs that do not hatch. If the proportion of sterile males consistently exceeds the proportion of fertile males then each new generation's reproduction is suppressed. As the wild population numbers dwindle, SIT becomes more and more effective creating a negative feedback loop that ultimately eradicates the species in an area. One major advantage of SIT population suppression versus traditional insecticide treatment is that it is species specific and environmentally safe. Three major processes are necessary for the implementation of SIT: 1) a method of sterilization; 2) a method of sex separation; and 3) a method of dispersal. The invention herein relates to the first point and represents a unique method of sterilization.

The historical example of SIT is Knipling's and the USDA's rearing of irradiated sterile males to eradicate the New World Screwworm (Cochliomya hominivorax) in North America and Mexico (Bushland, R. C., et al. Science 122, 287-288 (1955)). Screwworm is a deadly livestock pest which causes myiasis (an infestation of parasitic fly larvae that feed on host tissues)(Lindquist, D. A., et al. Med Vet Entomol 6, 2-8 (1992)). Initial field tests were carried out in Florida starting in 1951 and later in 1954 on the island of Curacao (Baumhover, A. H. et al. J Econ Entomol 48, 462-466 (1955)). This initial program utilized gamma rays of cobalt to sterilize male pupae (Bushland, R. C. & Hopkins, D. E. J Econ Entomol 44, 725-731 (1951)). Adult flies were then dispersed over the island by weekly release from an airplane. After 6 months of releases, screwworm was completely eradicated from the island (Baumhove. Ah. J Amer Med Assoc 196, 240 (1966)). Using the same technique, screwworm was eradicated from Florida and the Southeast USA by 1959 (Baumhove. Ah. J Amer Med Assoc 196, 240 (1966); Baumhover, A. H., et al. J Econ Entomol 52, 1202-1206 (1959)) and entirely from North and Central America by 1995 (Baumhover, A. H. Baumhover: A Personal Account of Screwworm Eradication. Pioneer Lecture presentation (1997)). SIT based eradication of the screwworm was later replicated in Libya (1990) when a shipment of contaminated livestock caused an outbreak; the technique has been proven to be a useful suppression tactic for many insects (Lindquist, D. A., et al. Med Vet Entomol 6, 2-8 (1992)).

The physical quality or “fitness” of sterile insects produced for SIT is of paramount importance for the application.⁹ One downside of canonical sterilization by irradiation is that many insects are not as resilient to this treatment as screwworm. For example, mosquitoes are more sensitive to irradiation and cannot be irradiated without significant fitness reductions and lethality (Benedict, M. Q. & Robinson, A. S. Trends Parasitol 19, 349-355 (2003); Dame, D. A., et al. Historical applications of induced sterilization in field populations of mosquitoes. Malaria J 8 (2009)). Thus, alternative means of sterilization are useful inventions for the development and application of SIT. Other methods of inducing sterility in insects include cytoplasmic incompatibility (CI), chromosomal disruptions, chemical sterilization, and sex ratio distortion (Benedict, M. Q. & Robinson, A. S. Trends Parasitol 19, 349-355 (2003)). CI is a conditional sterility induced by a secreted bacterial sperm toxin produced from Wolbachia infections in insect gonads (described above). Hannes Laven was the first to pioneer research on Wolbachia as a tool for SIT. He described how Culex pipiens mosquito isolates were sterile when mated with isolates from different regions of Europe (Laven, H. Chapter 7: Speciation and Evolution in Culex pipiens. 251 (Elselvier, 1967)). Realizing the potential, Laven isolated a strain of Culex pipiens fatigans (major vector of filariasis) which would be sterile when mated to the same species in Burma. Unbeknownst to Laven, his mosquito strain was infected with a corresponding strain of Wolbachia incompatible with the wild type populations in Burma. Despite not understanding the functionality of the sterility, Laven was able to use Wolbachia sterilized male mosquitoes to eradicate populations of the local mosquito vector in Burma (Laven, H. Nature 216, 383 (1967)).

Although the proof of principle has existed in the public domain with respect to Wolbachia mediated CI and SIT, it is important to note that the molecular mechanism and genetic system by which this happens had not been understood for over 60 years until the experiments described in this application were performed. Thus, the important distinction to be made between the invention disclosed herein and Wolbachia mediated SIT is that the inventors have identified the minimal molecular components from the Wolbachia genome that are sufficient to induce sterility by a transgenic means, independent of the Wolbachia bacterium. This last point importantly distinguishes the present invention from the invention described in U.S. Pat. No. 9,090,911 which describes a line of mosquito adapted by infection of variants of the Wolbachia strain wMel.

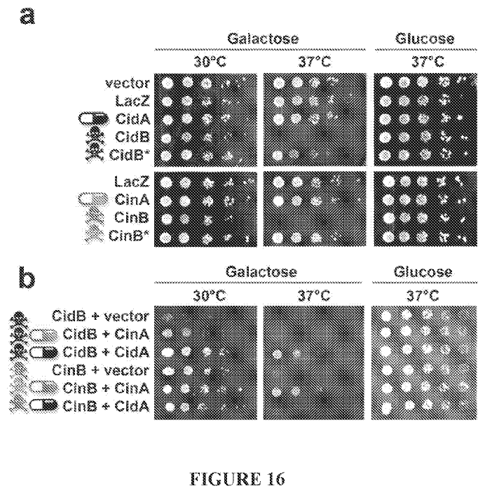

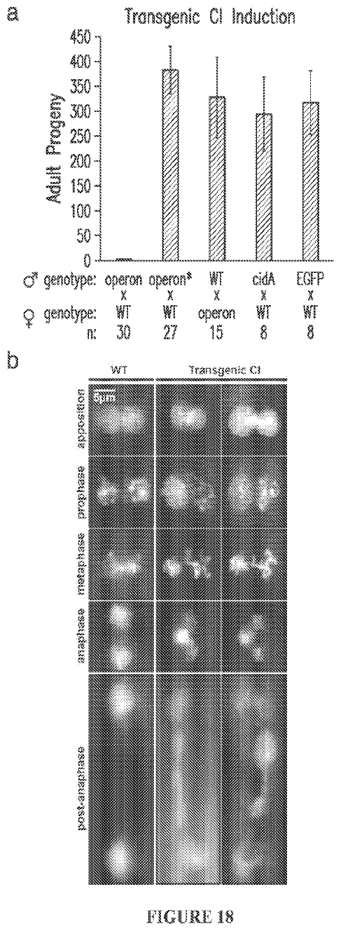

Therefore, the present application of these “bacterial operons” utilizes the cytoplasmic incompatibility genes or their derivatives within a construct able to be transgenically inserted into a pest insect for the purposes of inducing sperm sterilization. In one embodiment, the CidA/B^{wPip} operon is used as it induces extremely high levels of CI nearing 100% sterility in D. melanogaster (FIG. 18; See Example 2). In some embodiments, the cidA/B^{wMel} operon is used for application of SIT. Achieving a perfect 100% sterility is not entirely necessary for application of SIT (Dame, D. A., et al. Historical applications of induced sterilisation in field populations of mosquitoes. Malaria J 8 (2009)). Furthermore other “bacterial operons” including but not limited to CinA/B can be used in applications of SIT as the CinA/B operon was shown to exhibit toxin like properties in yeast comparable to the CidA/B operon (FIG. 16). In additional embodiments, many derivative “bacterial operons,” including but not limited to, the examples seen in FIGS. 6, 7, and 15, can be used for sterilization of insects.

After sterilization, male insects could then be separated from female insects, delivered to the target site, and released for mating with wild females to eradicate a pest population.

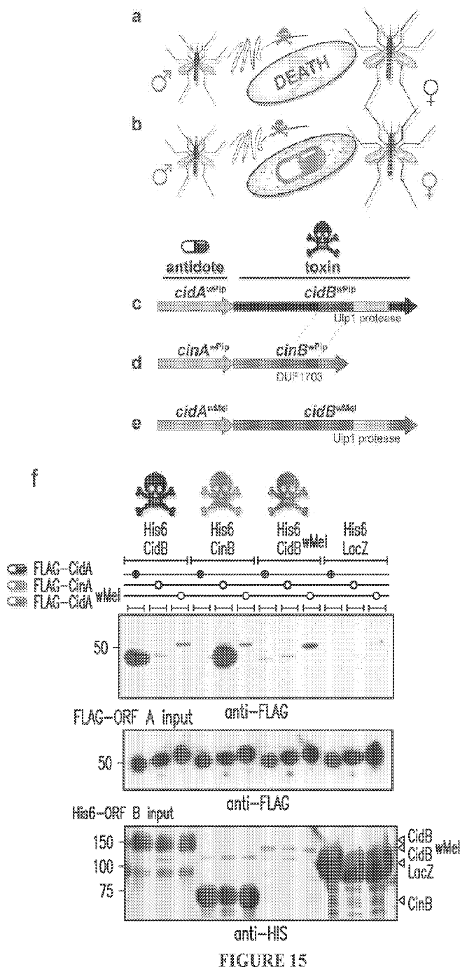

Alterations to the system can be made to optimize sterilization effectiveness of the “bacterial operon”. However, these optimizations do not change the essential composition of the “bacterial operon.” These changes might include but are not limited to: 1) alterations of gene regulatory sequences as sterility was induced utilizing various promoters such as the nanos promoter of the Gal4/UAS system described in Example 1 or the P-element promoter described in Example 2; 2) the insertion of protein affinity tags, post/pre-translational modifications, or untranslated exons altering detectability, stability, localization, or structure of the “bacterial operon” proteins or their transcripts as evidenced by sterility induction by a His6-V5 tagged version of CidA with a FLAG tagged version of CidB in the Example 2. Furthermore, In this case the mRNA of the bacterial operon was also stabilized and localized into the germline by the K10 3′ untranslated region of the last exon of the K10 gene (Rorth, P. Mech Dev 78, 113-118 (1998)); 3) Amino acids mutations/variants altering binding affinities between cognate operon pair proteins as FIG. 15F indicates that different amino acids regulate binding affinities of the cognate partner proteins; 4) alternative methods of driving expression of duel cognate partner proteins as two independent chromosomal insertions were utilized in Example 1 in contrast to an engineered eukaryotic operon with both open reading frames separated by the T2A insect peptide, which directs ribosomal translation of two separate proteins (Diao, F. & White, B. H. Genetics 190, 1139-1144 (2012)). 5) known transcriptional regulators including Gal4/UAS or others such as the tetracycline promoter and repressor for timed induction of expression.

The present method is uniquely different from other available genetic methods of sterilization such as Oxitec's patented RIDL technology (U.S. Pat. No. 9,125,388). In the cited patent and its published literature (Harris, A. F. et al. Nat Biotechnol 29, 1034-1037 (2011); Waltz, E. Nat Biotechnol 33, 792-793 (2015) an invention is described whereby biological control of an insect is achieved by the release of a dominant negative lethal gene under the control of transcriptional regulators. The unique difference with the method herein is the fact that the “bacterial operon” does not encode a dominant lethal gene. The “bacterial operons” instead sterilize sperm alone and effectively inhibit embryonic development and hatching of eggs. Evidence for this is provided in FIG. 18 (See Example 2) where sterility only occurs in “bacterial operon” transgenic males but not in transgenic females; also embryos resulting in crosses to “bacterial operon” transgenic males all die before egg hatch and exhibited extreme developmental deformities before larval emergence indicative of sperm defects. These phenotypes are in contrast to the dominant lethal approach which allows growth and development and only acts by killing larvae in a later developmental instar. Another distinction is that dominant lethal genes for SIT in one insect might not translate effectively into application for another genus of insect. The methods herein describe a cross compatible example of a platform technology capable of being applied to any insect genus. Proof of this lies in the fact that the “bacterial operon” CidA/B^{wPip} derived from Wolbachia which infects and induces CI in mosquitoes was able to induce sterility in fruit flies as well as toxicity in a model eukaryote yeast. Thus, the invention's effectiveness and application is not limited by species of insect and need not be restricted to Drosophila.

Once released, the transgenic insertion can be used as a tracking marker distinguishing the modified sterile insects from wild insects. Thus the “bacterial operons” would provide an additional tool to monitor and characterize the spread or incompatibility of the released populations or other populations of insects containing the “bacterial operons.” These markers can be detected by means such as standard polymerase chain reaction or antibody based detection. Furthermore, because the “bacterial operons” described underlie the reproductive barriers induced by wild strains of variant Wolbachias in insects, these markers can be used for determining and assessing mating compatibilities of any intraspecies insect strains in general. Thus, commercial testing, research, and reproductive compatibility assessment by characterization of these “bacterial operons” can be used as a pest management tool for agricultural companies seeking to eradicate or monitor the spread of a particular pest.

A separate application independent of SIT, but inherently related to sterilization of insect sperm is the sterilization of transgenic strains of insects for safety testing. Newly created strains of genetically modified organisms (GMOs) are able to prevent or repress the transmission of diseases like malaria (Ito, J., et al. Nature 417, 452-455 (2002); Jacobs-Lorena, M. J Vector Borne Dis 40, 73-77 (2003)). However, release of such GMO insects cannot be performed without substantial field and safety testing. Initial field tests are often first administered by sterilizing the GMO insects before release such that they will not pass on modified chromosomes onto the next generation in the wild (Benedict, M. Q. & Robinson, A. S. Trends Parasitol 19, 349-355 (2003)). This allows safe examination of off-target effects of GMOs. Thus, in additional methods disclosed herein, the bacterial operons can be used to sterilize the GMO for safety tests.

In one aspect, provided herein is a genetically modified arthropod, said arthropod comprising:
a bacterial operon encoding a cytoplasmic incompatibility factor or a variant thereof; and
a promoter operably linked to the bacterial operon;
wherein the expression of the cytoplasmic incompatibility factor in a male arthropod causes a reduction in viable offspring in comparison to a male arthropod lacking the cytoplasmic incompatibility factor.

In another aspect, provided herein is a method for controlling a population of target arthropods, comprising:
providing a bacterial operon encoding a cytoplasmic incompatibility factor or a variant thereof, and a promoter operably linked to the bacterial operon;
transforming a population of male arthropods with the bacterial operon; and
releasing the male arthropods amongst a population of target arthropods, wherein the release of the male arthropods reduces the population of target arthropods.

In one embodiment, the bacterial operon is from Wolbachia. In one embodiment, the bacterial operon is from wMel. In one embodiment, the bacterial operon encodes the cytoplasmic incompatibility factor WD0631. In one embodiment, the bacterial operon encodes the cytoplasmic incompatibility factor WD0632. In one embodiment, the bacterial operon encodes the cytoplasmic incompatibility factors WD0631 and WD0632.

In one embodiment, the bacterial operon is from Wolbachia pipientis. In one embodiment, the bacterial operon encodes the cytoplasmic incompatibility factor CidA^{wPip} (wPa_0282). In one embodiment, the bacterial operon encodes the cytoplasmic incompatibility factor CidB^{wPip} (wPa_0283). In one embodiment, the bacterial operon encodes the cytoplasmic incompatibility factors CidA^{wPip} (wPa_0282) and CidB^{wPip} (wPa_0283). In one embodiment, the bacterial operon encodes the cytoplasmic incompatibility factor CinA^{wPip} (wPa_0294). In one embodiment, the bacterial operon encodes the cytoplasmic incompatibility factor CinB^{wPip} (wPa_0295). In one embodiment, the bacterial operon encodes the cytoplasmic incompatibility factors CinA^{wPip} (wPa_0294) and CinB^{wPip} (wPa_0295).

In one embodiment, the bacterial operon is from Cardinium. In one embodiment, the bacterial operon is from Rickettsia.

In one embodiment, the bacterial operon encodes a deubiquitylase. In one embodiment, the bacterial operon encodes a nuclease.

In one embodiment, the reduction in viable offspring is greater than 50%. In one embodiment, the reduction in viable offspring is greater than 60%. In one embodiment, the reduction in viable offspring is greater than 70%. In one embodiment, the reduction in viable offspring is greater than 80%. In one embodiment, the reduction in viable offspring is greater than 90%. In one embodiment, the reduction in viable offspring is greater than 95%.

In one embodiment, the arthropod is an insect. In one embodiment, the insect is selected from the genera consisting of Aedes, Culex and Anopheles. In one embodiment, the insect is selected from the group consisting of Aedes albopictus, Aedes aegypti and Aedes polynesiensis. In one embodiment, the insect is Drosophila suzukii.

### Methods of Controlling Arthropod Populations: Population Replacement

Another method for controlling pest and disease vector populations is a Population Replacement Strategy (PRS). Its goal is to replace wild pest or vector populations with those that are not competent to function as pests or vectors of human disease (Sinkins, 2004, Insect Biochem Mol Biol, 34, 723-9; Dobson, Brelsfoard and Dobson, 2009, AsPac J. Mol. Biol. Biotechnol., 17, 55-63). Population Replacement is dependent on two pieces of technology:
1) A beneficial trait that is desired in the target arthropod
2) A genetic drive mechanism to spread the desired trait through the arthropod population (Sinkins and Gould, 2006, Nat Rev Genet, 7, 427-35).
   The technology described by the inventors in the present disclosure addresses the second problem by utilizing bacterial operons that induce cytoplasmic incompatibility (CI), which is a natural genetic drive mechanism used by various, unrelated bacterial infections (e.g., Wolbachia and Cardinium endosymbionts).

A previously used approach involving population replacement in the control of disease vectors is the Eliminate Dengue project. This method uses the naturally occurring Wolbachia strain wMel to introduce both a desirable trait, the inhibition of mosquito vector competence for Dengue virus and other human pathogens (Walker et al., 2011, Nature, 476, 450-3; Aliota et al., 2016, PLOS Negl Trop Dis, 10, e0004677; Dutra et al., 2016, Cell Host Microbe), and the genetic drive mechanism of CI. This technique has had limited success in field trials, but requires massive mosquito releases (Hoffmann et al., 2011, Hoffmann et al., 2014) and the horizontal transfer of Wolbachia into hosts that are frequently inhospitable to stable infection (Hughes et al., 2011, PLOS Pathog, 7, e1002043; Hughes et al., 2014, Proc Natl Acad Sci U S A, 111, 12498-503).

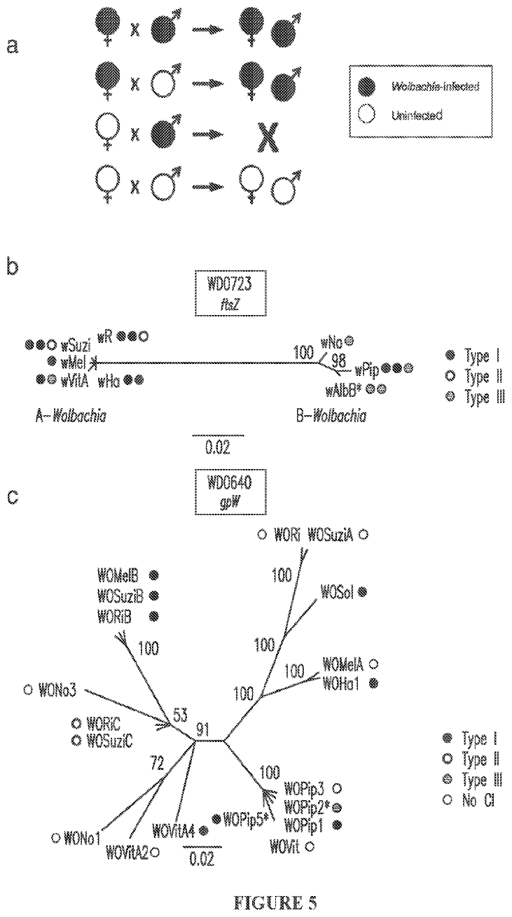

Disclosed herein are methods for population replacement. The first includes generating CI-inducing males that do not harbor the classical bacteria required for CI such as Wolbachia (Zabalou et al., 2004, Proc Natl Acad Sci USA, 101, 15042-5) or Cardinium (Gotoh et al., 2007, Heredity (Edinb), 98, 13-20; Penz et al., 2012, PLOS Genet, 8, e1003012) species. Historically, one of the major hurdles to utilizing a population replacement strategy has been the difficulty of transferring these CI-inducing organisms into new host species (Hughes et al., 2011, PLOS Pathog, 7, e1002043; Hughes et al., 2014, Proc Natl Acad Sci USA, 111, 12498-503). In fact, only three in 2,541 attempts led to transinfected Aedes aegypti after two years of cell-line adaptation (Walker et al., 2011, Nature, 476, 450-3). Moreover, once released in the population, the infection has to efficiently vertically transmit itself to the next generation and avoid the evolution of host suppression traits that eliminate the bacteria (Rasgon, 2008, Adv Exp Med Biol, 627, 114-25). The proposed technology circumvents these concerns by transgenically inserting the bacterial operons or their derivatives directly into the host nuclear genome, cytoplasmic genome (e.g., mitochondria), or into the genomes of various host-associated microorganisms (i.e., bacteria, viruses, archaea, protists) that are vertically inherited from parents to offspring. This technology would not be limited to just a handful of species, as Example 2 shows that bacterial operons derived from the Wolbachia infection of Culex pipiens are also effective in Drosophila. Further, FIGS. 5, 6, and 15 show examples of closely related bacterial operons that could be utilized in a broad range of animal species. This alleviates the current issues with inducing CI in novel hosts. It is important to note that utilizing CI bacterial operons instead of the CI-inducing bacteria relies on transgenic insertion of both the CI and “rescue” genes.

Some current uses of a PRS rely on one factor, such as an infection by Wolbachia pipientis, to provide both the beneficial trait and the genetic drive mechanism required for population replacement. The use of bacterial operons to induce CI, however, is an improvement to this approach as it de-couples the genetic drive mechanism from the desired trait being spread. This allows for a larger assortment of traits to be spread through PRS as they do not have to be provided by a technology or organism, such as a naturally occurring Wolbachia infection, that also induces CI. The bacterial operons could thus be utilized in conjunction with other technologies that may alter host fitness, lifespan, or disease resistance to propagate different desired traits through a population.

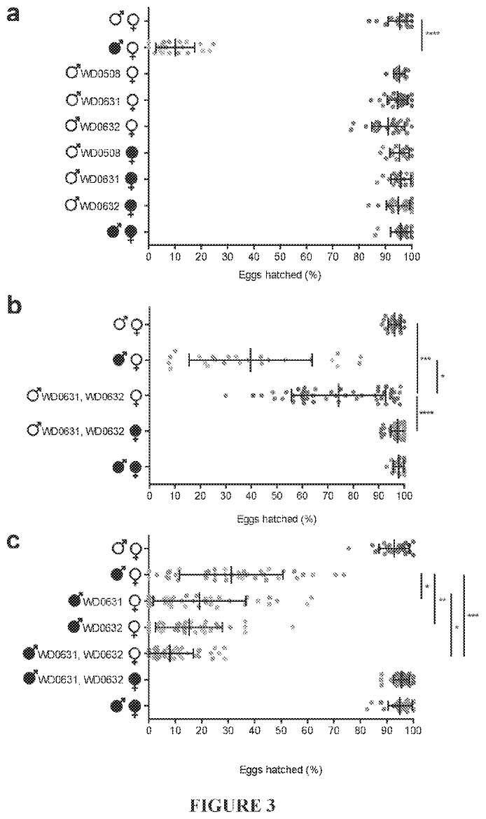

A second option disclosed herein is to utilize the bacterial operons in conjunction with current approaches. The current technology is described in U.S. Pat. No. 9,090,911 but, importantly, is reliant upon CI induced by Wolbachia strains that are also detrimental to the host (wMel-Pop (Nguyen et al., 2015, Parasit Vectors, 8, 563; Ritchie et al., 2015, PLOS Negl Trop Dis, 9, e0003930)) or which induce incomplete CI (wMel (Reynolds and Hoffmann, 2002, Genet Res, 80, 79-87)). In one embodiment, bacterial operon products are expressed within these animals by inserting the genes into the host nuclear genome, cytoplasmic genome (e.g., mitochondria), or into the genomes of various host-associated microorganisms, including Wolbachia. FIG. 3 (See Example 1) shows that expression of bacterial operon genes in Wolbachia-infected insects is able to increase the rate of CI. Further, bacterial operon genes have not been shown to be detrimental to hosts. This means that current approaches can be enhanced through usage of bacterial operons as CI would be stronger without sacrificing host health. This can greatly increase the rate of population replacement and reduce the number of released animals required (Chan and Kim, 2013, Bull Math Biol, 75, 1501-23; Engelstadter and Telschow, 2009, Heredity (Edinb), 103, 196-207).

Several unfavorable aspects of current population replacement strategies are that the new population may lose its beneficial qualities (through mutation, adaptation, or some other process) or may become actively harmful (through mosquito over proliferation, enhancement of replication of other microbes including malaria (Hughes et al., 2014, PLOS Pathog, 10, e1004182) or West Nile virus (Dodson et al., 2014, PLOS Negl Trop Dis, 8, e2965), or acquired traits). In this situation the proposed bacterial operons can also be utilized to spread a new replacement strain. It is well established that, in the natural context, CI induced by Wolbachia pipientis is strain specific (Sinkins, 2004, Insect Biochem Mol Biol, 34, 723-9). It becomes possible then to utilize differential versions of the bacterial operons, such as those in FIGS. 6, 7, and 15, to perform multiple rounds of population replacement with new CI/rescue factor combinations, wherein the new strains induce a version of CI that cannot be rescued by the incumbent population. This provides a measure of control over the previously released populations and also allows for new benefits to be introduced as technology advances.

Alterations to the system can be made to optimize effectiveness of the “bacterial operon”, as discussed in the section above discussing the sterile insect technique. Additionally, once released, the transgenic insertion can be used as a tracking marker distinguishing the insects containing the genetically modified bacterium from wild insects, as further discussed in the section above discussing the sterile insect technique.

Previous examples of population replacement strategies include using wMel or wMel-Pop in mosquitoes (U.S. Pat. No. 9,090,911), recombinant insect with dominant lethal gene (U.S. Pat. No. 9,125,388): wMel provided disease resistance (WO2013026994): transferring Wolbachia to induce CI (WO2006008652): using transformed Wolbachia for similar techniques (WO1994002591): see also U.S. Pat. No. 7,868,222.

Additional patents that discuss methods for gene drives and population replacement strategies include for example, WO2015105928, and WO2013131920A1, which include methods using homing endonucleases such as zinc finger nucleases (ZFNs), transcription activator-like effector nucleases (TALENs), and CRISPR systems, such as the CRISPR/Cas9 and CRISPR/Cpf1 systems. These gene drive systems may be used in combination with the bacterial operons disclosed herein (for example, encoding cytoplasmic incompatibility factors).

In one aspect, provided herein is a genetically modified bacterium comprising:
a bacterial operon encoding a cytoplasmic incompatibility factor or a variant thereof; and
a promoter operably linked to the bacterial operon;
wherein the bacterial operon occurs at a non-naturally occurring genomic location in the bacterium.

In another aspect, provided herein is an arthropod infected with a bacterium, wherein the bacterium comprises:
a bacterial operon encoding a cytoplasmic incompatibility factor or a variant thereof; and
a promoter operably linked to the bacterial operon;
wherein the bacterial operon occurs at a non-naturally occurring genomic location in the bacterium.

In an additional aspect, provided herein is a method for controlling a population of target arthropods, comprising:
providing a genetically modified bacterium comprising:
   a bacterial operon encoding a cytoplasmic incompatibility factor or a variant thereof, and
   a promoter operably linked to the bacterial operon;
infecting a population of replacement arthropods with the genetically modified bacterium; and
releasing the replacement arthropods amongst a population of target arthropods, wherein the release of the replacement arthropods reduces the population of target arthropods.

In one embodiment, the bacterial operon is from Wolbachia. In one embodiment, the bacterial operon is from wMel. In one embodiment, the bacterial operon encodes the cytoplasmic incompatibility factor WD0631. In one embodiment, the bacterial operon encodes the cytoplasmic incompatibility factor WD0632. In one embodiment, the bacterial operon encodes the cytoplasmic incompatibility factors WD0631 and WD0632.

In one embodiment, the bacterial operon is from Wolbachia pipientis. In one embodiment, the bacterial operon encodes the cytoplasmic incompatibility factor CidA wPip (wPa_0282). In one embodiment, the bacterial operon encodes the cytoplasmic incompatibility factor CidB^{wPiP} (wPa_0283). In one embodiment, the bacterial operon encodes the cytoplasmic incompatibility factors CidA^{wPip} (wPa_0282) and CidB^{wPip} (wPa_0283). In one embodiment, the bacterial operon encodes the cytoplasmic incompatibility factor CinA wPip (wPa_0294). In one embodiment, the bacterial operon encodes the cytoplasmic incompatibility factor CinB^{wPip} (wPa_0295). In one embodiment, the bacterial operon encodes the cytoplasmic incompatibility factors CinA^{wPip} (wPa_0294) and CinB^{wPip} (wPa_0295).

In one embodiment, the bacterial operon is from Cardinium. In one embodiment, the bacterial operon is from Rickettsia.

In one embodiment, the bacterial operon encodes a deubiquitylase. In one embodiment, the bacterial operon encodes a nuclease.

In one embodiment, the bacterium is Wolbachia. In one embodiment, the bacterium is Cardinium. In one embodiment, the bacterium is Rickettsia.

In one embodiment, the arthropod is an insect. In one embodiment, the insect is selected from the genera consisting of Aedes, Culex and Anopheles. In one embodiment, the insect is selected from the group consisting of Aedes albopictus, Aedes aegypti and Aedes polynesiensis. In one embodiment, the insect is Drosophila suzukii.

### Arthropods and Infectious Disease Vectors

The inventors have identified a primary mechanism for CI involving cytoplasmic incompatibility factor proteins secreted into germline cells by resident bacteria and disclose herein new methods for control of arthropod (for example, insects) pests and disease vectors, such as mosquitoes transmitting the Dengue fever and Zika viruses.

In one embodiment, the arthropod is an insect. In one embodiment, the arthropod is a mosquito. In one embodiment, the mosquito is selected from the genera consisting of Aedes, Culex and Anopheles. In one embodiment, the mosquito is an Aedes mosquito. In one embodiment, the mosquito is an Anopheles mosquito. In one embodiment, the mosquito is a Culex mosquito. In one embodiment, the Aedes mosquito species is selected from the group consisting of Aedes albopictus, Aedes aegypti and Aedes polynesiensis. In one embodiment, the Anopheles mosquito species is Anopheles gambiae. In one embodiment, the Culex mosquito species is Culex pipiens.

In one embodiment, disclosed herein are methods for controlling or reducing populations of insects that transmit human or veterinary pathogens. In one embodiment, disclosed herein are methods for replacing a population of arthropods that transmit human or veterinary pathogens with a replacement arthropod population that is infected with a genetically modified bacteria (for example Wolbachia) that reduces the ability of the insect to transmit the pathogen. In one embodiment, the pathogen is selected from dengue virus, Zika virus, a malaria parasite (Plasmodium genus), West Nile virus, yellow fever virus, chikungunya virus, Japanese encephalitis, St. Louis encephalitis and Western and Eastern Equine Encephalitis viruses.

In one embodiment, disclosed herein are methods for controlling or reducing populations of insects that transmit trypanosomes including African sleeping sickness, Chagas disease, and Nagana. In one embodiment, the pathogen is Trypanosoma cruzi. In one embodiment, the pathogen is Trypanosoma brucei. In one embodiment, the insect is of the genus Glossina. In one embodiment, the insect is Glossina morsitans. In one embodiment, the insect is a Tsetse fly. In one embodiment, the insect is a kissing bug. In one embodiment, the insect is of the genus Rodnius. In one embodiment, the insect is Rhodnius prolixus.

In one embodiment, disclosed herein are methods for controlling or reducing populations of arthropods that transmit rickettsioses and pathogens within Anaplasmatacea including Rickettsias rickettsii, africae, parkeri, sibirica, conorii, slovaca, peacockii, philipii, rickettsii Hlp2, heilongjiangensis, japonica, montanensis, massiliae, rhipicephali, amblyommii, helvetica, monacensis, buchneri, hoogstralli, felis, akari, australis, canadensis, prowazekii, typhi, bellii. In one embodiment, the arthropod is a tick. In one embodiment, the arthropod is a tick of the genera Amblyomma, Ixodes, or Rhipicephalus. In one embodiment, the disease is epidemic typhus. In one embodiment, the disease is scrub typhus. In one embodiment, the disease is an Ehrlichiosis. In one embodiment, the pathogen is of the genus Ehrlichia. In one embodiment, the pathogen is of the genus Anaplasma. In one embodiment, the pathogen is of the genus Orientia. In one embodiment, the arthropod is a chigger of the genus Leptotrombidium. In one embodiment, the arthropod is a louse of the genus Pediculus. In one embodiment, the arthropod is a flea of the genus Pulex.

In one embodiment, the invention is useful for controlling sandflies that transmit leishmaniasis. In one embodiment, the insect is of the genus Phlebotomus. In one embodiment, the pathogen is of the genus Leishmania. In one embodiment, the pathogen is Leishmania donovani, Leishmania infantum, or Leishmania Chagasi.

In one embodiment, the insect is of various aphids including: Acyrthosiphon kondoi, Brevicoryne brassicae, Rhopalosiphum maidis, Aphis gossypii, Aphis craccivora, Myzus persicae, Rhopalosiphum padi, Acyrthosiphon pisum, Rhopalosiphum rufiabdominalis, Metopolophium dirhodum, Aphis glycine, Therioaphis trifolii, Lipaphis erysimi, Rhopalosiphum padi.

In one embodiment, the invention is useful for controlling the armyworm agricultural pest species including Leucania convecta, Spodoptera exempta, Spodoptera Mauritia, Spodoptera exigua, Mythimna separate, Leucania stenographa.

In one embodiment, the invention is useful for controlling pests of beans and beets. In one embodiment, the insect is either the Bean fly (Ophiomyia phaseoli), the Bean leafroller (Omiodes diemenalis), the Bean looper or Mocis (Mocis alterna), the Bean podborer (Maruca vitrata), the Bean spider mite (Tetranychus ludeni), the Beet webworm (Spoladea recurvalis), the Large Brown bean bug (Riptortus serripes), the Small Brown bean bug (Melanacanthus scutellaris)

In one embodiment, the invention is useful for controlling the Blue oat mite (Penthaleus major). In one embodiment, the invention is useful for controlling the Brown flea beetle (Chaetocnema sp.). In one embodiment, the invention is useful for controlling the Brown mirid (Creontiades pacificus). In one embodiment, the invention is useful for controlling the Brown shield bug (Dictyotus caenosus). In one embodiment, the invention is useful for controlling the Brown wheat mite (Petrobia latens). In one embodiment, the invention is useful for controlling the Bruchid, Cowpea (Callosobruchus maculatus).

In one embodiment, the invention is useful for controlling pests of Corn including: the Corn aphid (Rhopalosiphum maidis), and the Corn earworm (Helicoverpa armigera).

In one embodiment, the invention is useful for controlling pests of cotton including the Cotton aphid (Aphis gossypii), Cotton bollworm (Helicoverpa armigera), the Cotton harlequin bug (Tectocoris diophthalmus), the Cotton leafhopper (Amrasca terraereginae), the Cotton leafperforator (Bucculatrix gossypii), the Cotton looper (Anomis flava), the Cottonseed bug (Oxycarenus luctuosus), the Cotton seedling thrip (Thrips tabaci), the Cotton tipworm (Crocidosema plebejana), and the Cotton webspinner (Achyra affinitalis).

In one embodiment, the invention is useful for controlling the Diamondback moth (Plutella xylostella). In one embodiment, the invention is useful for controlling the Dried fruit beetle (Carpophilus spp.). In one embodiment, the invention is useful for controlling the Eastern false wireworm (Pterohelaeus spp.). In one embodiment, the invention is useful for controlling the Etiella moth (Etiella behrii). In one embodiment, the invention is useful for controlling the False wireworm (Pterohelaeus and Gonocephalum spp.). In one embodiment, the invention is useful for controlling the Flea beetles, Brown and Redheaded (Chaetocnema and Nisostra sp.). In one embodiment, the invention is useful for controlling the Flower beetle (Carpophilus spp.).

In one embodiment, the invention is useful for controlling various Grasshoppers and locusts including the Grasshopper, Wingless (Phaulacridium vittatum), the Locust, Australian plague (Chortoicetes terminifera), the Locust, Migratory (Locusta migratoria), the Locust, Yellow-winged (Gastrimargus musicus), the Locust, Spur-throated (Austracris (Noamdacris) guttulosa).

In one embodiment, the invention is useful for controlling the Greenhouse whitefly (Trialeurodes vaporariorum). In one embodiment, the invention is useful for controlling the Green peach aphid (Myzus persicae). In one embodiment, the invention is useful for controlling the Green mirid (Creontiades dilutus). In one embodiment, the invention is useful for controlling the Green vegetable bug (Nezara viridula). In one embodiment, the invention is useful for controlling the Green stink bug (Plautia affinis). In one embodiment, the invention is useful for controlling the Grey cluster bug (Nysius clevelandensis). In one embodiment, the invention is useful for controlling the Helicoverpa species (armigera and punctigera).

In one embodiment, the invention is useful for controlling planthoppers. In one embodiment, the insect is the small brown planthopper (Laodelphax striatellus). In one embodiment, the invention is useful for preventing the transmission of crop diseases like Rice White Stripe Virus. In one embodiment, the invention is useful for controlling vectors of plant pathogens.

In one embodiment, the invention is useful for controlling the Jassids and various leafhoppers including the Leafhopper, cotton (Amrasca terraereginae), the Leafhopper, lucerne (Austroasca alfalfae), the Leafhopper, maize (Cicadulina bimaculata), the Leafhopper, vegetable (Austroasca viridigrisea).

In one embodiment, the invention is useful for controlling the Loopers including the Looper, Brown pasture (Ciampa arietaria), the Looper, Castor oil (Achaea janata), the Looper, Cotton (Anomis flava), the Looper, Sugarcane (Mocis frugalis), the Looper, Soybean (Thysanoplusia orichalcea), the Looper, Tobacco (Chrysodeixis argentifera), the Looper, Vegetable (Chrysodeixis eriosoma).

In one embodiment, the invention is useful for controlling various Thrip pests including the Onion Thrip (Thrips tabaci), the Cotton seedling Thrip (Thrips tabaci), the Maize Thrip (Frankliniella williamsi), the Plague Thrip (Thrips imaginis), the tobacco Thrip (Thrips tabaci), the Tomato Thrip (Frankliniella schultzei), the Western flower Thrip (Frankliniella orientalis)

In one embodiment, the invention is useful for controlling various Mite pests including the Mite, Bean spider (Tetranychus ludeni), Mite, Brown wheat (Petrobia latens), Mite, Blue oat (Penthaleus major), Mite, Peanut (Paraplonobia spp.), Mite, Redlegged earth (Halotydeus destructor), Mite, Strawberry spider (Tetranychus lambi), and the Two-spotted mite (Tetranychus urticae).

In one embodiment, the invention is useful for controlling various whitefly pests including the Greenhouse whitefly (Trialeurodes vaporariorum), the Silverleaf whitefly (Bemisia tabaci biotype B and Australian native AN), and the Silverleaf whitefly (Bemisia tabaci biotype Q).

In one embodiment, the inventions is useful for controlling various fruit pests. In one embodiment, the arthropod is from the genera Drosophila. In one embodiment, the arthropod is Drosophila suzukii. Drosophila suzukii, commonly called the spotted-wing drosophila, is a vinegar fly closely related to Drosophila melanogaster. Unlike its vinegar fly relatives who are primarily attracted to rotting or fermented fruit, D. suzukii attacks fresh, ripe fruit by laying eggs under the soft skin. The larvae hatch and grow in the fruit, destroying the fruit's commercial value. The pest particularly (but not limited to) infests cherries, apples, apricots, persimmons, tomatoes, blueberries, grapes, nectarines, pears, plums, peaches, figs, raspberries and strawberries. Although D. suzukii is native to Southeast Asia, the fruit pest has recently invaded North and Central America as well as Europe, where it is expanding rapidly. Effective management of this pest is a challenge owing to the wide host range and short generation time. Therefore, monitoring and controlling D. suzukii is of great economic importance. However, traps and baits containing for instance apple cider vinegar, which are typically used for attracting vinegar flies such as D. melanogaster, are less efficient for attracting and trapping D. suzukii. In one embodiment, the insect is the Mexican Fruit Fly (Anastrepha ludens). In one embodiment, the insect is the Mediterranean Fruit Fly (Ceratitis capitata). In one embodiment, the insect is of the genus Anastrepha, Bactrocera, or Ceratitis. In one embodiment, the insect is a tephritid.

In one embodiment, the invention is useful for controlling various other agricultural pests including: the red-houldered leaf beetle (Monolepta australis), Native budworm (Helicoverpa punctigera), Native whitefly (Bemisia tabaci), Northern armyworm (Mythimna separata), Oat aphid (Rhopalosiphum padi), Onion thrip (Thrips tabaci), Pale cotton stainer bug (Dysdercus sidae), Pea aphid (Acyrthosiphon pisum), Pea blue butterfly (Lampides boeticus),

Peanut mite (Paraplonobia spp.), Peanut scarab (Heteronyx spp.), Pea weevil (Bruchus pisorum), Pinkspotted bollworm (Pectinophora scutigera), Plague thrip (Thrips imaginis), Podsucking bugs (Nezara viridula), Redbanded shield bug (Piezodorus oceanicus), Redheaded flea beetle (Nisotra sp.), Redlegged earth mite (Halotydeus destructor), Redshouldered leaf beetle (Monolepta australis), Rice root aphid (Rhopalosiphum rufiabdominalis), Rose grain aphid (Metopolophium dirhodum), Rough bollworm (Earias huegeliana), Rutherglen bug (Nysius vinitor), Seed harvesting ants (Pheidole spp.), Scarab, Black sunflower (Pseudoheteronyx sp.), Scarab, Peanut (JPG, 20.4 KB) (Heteronyx sp.), Shoot flies (Atherigona sp.), Silverleaf whitefly (Bemisia tabaci biotype B and Australian native AN), Silverleaf whitefly (Bemisia tabaci biotype Q), Sitona weevil (Sitona discoideus), Solenopsis mealybug (Phenacoccus solenopsis), Sorghum midge (Stenodiplosis sorghicola), Sorghum head caterpillar (Cryptoblabes adoceta), Soybean leafminer (Porphyrosela aglaozona), Soybean looper (Thysanoplusia orichalcea), Soybean moth (Aproaerema simplexella), Spotted alfalfa aphid (Therioaphis trifolii), Spur-throated locust (Austracris (Nomadacris) guttulosa), Strawberry spider mite (Tetranychus lambi), Swarming leaf beetle (Rhyparida spp.), Tortrix (Epiphyasa postvittana), True wireworm (Agrypnus spp.), Vegetable weevil (Listroderes difficilis), Weed web moth (Achyra affinitalis), Whitegrub (Heteronyx spp.), Wingless cockroaches (Calolampra spp.), Wireworm, False (Pterohelaeus and Gonocephalum spp.), Wireworm, True (Agrypnus spp.), Yellow peach moth (Conogethes punctiferalis). In one embodiment, the insect is Heteronychus arator. In one embodiment, the insect is of the genus Amnemus. In one embodiment, the insect is of the genus Pheidole. In one embodiment, the invention is useful for controlling the Black field cricket (Teleogryllus commodus, T. oceanicus, Lepidogryllus parvulus), the Black field earwig (Nala lividipes), the Black leaf beetle (Rhyparida nitida), the Black sunflower scarab (Pseudoheteronyx sp.). In one embodiment, the invention is useful for controlling the Cowpea bruchid (Callosobruchus maculatus). In one embodiment, the invention is useful for controlling the Cricket, Black field (Teleogryllus commodus, T. oceanicus, Lepidogryllus parvulus). In one embodiment, the invention is useful for controlling the Crop mirid (Sidnia kinbergi). In one embodiment, the invention is useful for controlling the Cutworm (Agrotis spp.). In one embodiment, the invention is useful for controlling the Cabbage moth (Plutella xylostella). In one embodiment, the invention is useful for controlling the Castor oil looper (Achaea janata). In one embodiment, the invention is useful for controlling the Click beetle (Agrypnus spp.). In one embodiment, the invention is useful for controlling the Clover springtail (Sminthurus viridis). In one embodiment, the invention is useful for controlling the Cluster caterpillar (Spodoptera litura). In one embodiment, the invention is useful for controlling the Cockroach, Wingless (Calolampra spp.). In one embodiment, the invention is useful for controlling the Common grass blue butterfly (Zizina labradus). In one embodiment, the invention is useful for controlling the Legume webspinner (Omiodes diemenalis). In one embodiment, the invention is useful for controlling the Light brown apple moth (Epiphyas postvittana). In one embodiment, the invention is useful for controlling Mocis trifasciata. In one embodiment, the invention is useful for controlling Pantydia spp. In one embodiment, the invention is useful for controlling the Lucerne crownborer (Zygrita diva). In one embodiment, the invention is useful for controlling the Lucerne flea (Sminthurus viridis). In one embodiment, the invention is useful for controlling the Lucerne leafhopper (Austroasca alfalfae). In one embodiment, the invention is useful for controlling the Lucerne leafroller (Merophyas divulsana). In one embodiment, the invention is useful for controlling the Lucerne seed wasp (Bruchophagus roddi). In one embodiment, the invention is useful for controlling the Lucerne seed web moth (Etiella behrii).

In one embodiment, the invention is useful for controlling forestry and wildlife pests such as the emerald ash borer. In one embodiment, the insect is of the genus Agrilus or specifically Agrilus planipennis. In one embodiment, the invention is useful for pests of trees and lumber.

### Examples

The following examples are set forth below to illustrate the results and methods according to the disclosed subject matter. These examples are not intended to be inclusive of all aspects of the subject matter disclosed herein, but rather to illustrate representative results and methods. These examples are not intended to exclude equivalents and variations of the present invention which are apparent to one skilled in the art.

### Example 1. Wolbachia-Induced Cytoplasmic Incompatibility is Caused by Prophage WO Genes

The genus Wolbachia is an archetype of maternally inherited intracellular bacteria that infect the germline of millions of invertebrate species worldwide and parasitically alter arthropod sex ratios and reproductive strategies to increase the proportion of infected females (the transmitting sex) in the population. The most common of these reproductive manipulations is cytoplasmic incompatibility (CI), typically expressed as embryonic lethality in crosses between infected males and uninfected females. This lethality is completely rescued by females infected with the same or a similar Wolbachia strain. Despite more than 40 years of research¹, the genes by which Wolbachia cause CI remain unknown. Here, we use comparative genomic, transcriptomic, proteomic and transgenic approaches to elucidate two genes that are CI effectors. In the Wolbachia strain wMel, the phage WO2-encoded operon consisting of WD0631 and WD0632 recapitulates significant degrees of CI in transgenic male Drosophila melanogaster that express both genes. The transgene-induced CI causes cytological defects similar to wild type CI, and it is fully rescued by wMel-infected females. The discovery of these two cytoplasmic incompatibility factor genes (cifA and cifB) represents an important step forward in understanding the genetics of reproductive parasitism and has implications for symbiont-induced speciation^{3,4} and control of agricultural pests⁵ and disease vectors that spread dengue virus^{6,7}, Zika virus⁸, and other human pathogens.

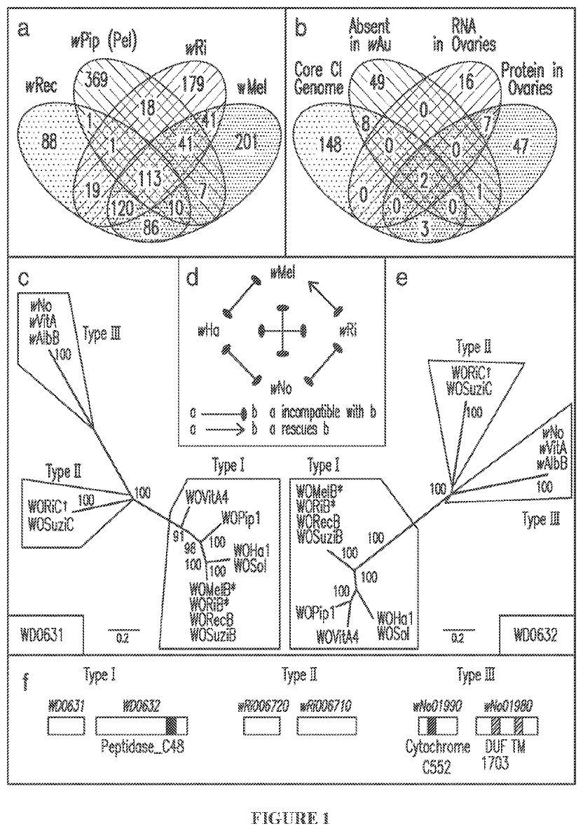

We hypothesized that the genes responsible for CI (FIG. 5a) would be present in all CI-inducing Wolbachia strains but absent or divergent in strains that are mutualists or that do not induce CI; we also predicted that these genes would be relatively highly expressed in the gonads of infected insects. To elucidate CI effector candidates, we determined the core genome shared by the CI-inducing Wolbachia strains wMel, wRi, wPip (Pel), and the recently sequenced wRec, which helped narrow the list of candidate prophage WO genes associated with reproductive parasitism⁹, while excluding the pan-genome of the mutualistic strain wBm. This analysis yielded 113 gene families representing 161 unique wMel genes (FIG. 1a, Supplementary Information 1a). Next we streamlined this candidate list by comparing it to (i) homologs of genes previously determined by comparative genomic hybridization to be absent or divergent in the strain wAu¹⁰, which does not induce CI, (ii) homologs to genes that are highly expressed at the RNA level in wVitA-infected Nasonia vitripennis ovaries, and (iii) homologs detected at the protein level in wPip (Buckeye)-infected ovaries of Culex pipiens mosquitoes. Remarkably, only two genes, those whose wMel locus tags are WD0631 and WD0632, were shared among all four gene subsets (FIG. 1b, Supplementary Information 1b-d). Notably, the homolog of WD0631 in the Wolbachia strain wPip, wPa_0282, was found at the protein level in the fertilized spermathecae of infected mosquitoes, lending support to the gene's role in reproductive manipulation¹¹.

We analyzed the evolution and predicted protein domains of these two genes and found that homologs of both genes are always associated with prophage WO in the Wolbachia chromosome¹², and they codiverged into three distinct phylogenetic groups that we designate type I, II, and III (FIG. 1c, e, Supplementary Information 1e). These relationships are not recapitulated in the phylogeny of the Wolbachia cell division gene ftsZ, which exhibits the typical bifurcation of A and B Wolbachia (FIG. 5b), or in the phylogeny of phage WO baseplate assembly gene gpW (FIG. 5c). This suggests that WD0631 and WD0632 are evolving under different evolutionary pressures than the core Wolbachia genome and active phage WO haplotypes.

Type I genes are the most prevalent amongst sequenced Wolbachia strains, and are always associated with large but incomplete phage WO regions that are missing important tail genes likely needed for active phage (FIG. 6). Although the function of type I WD0631 homologs are unknown, type I WD0632 homologs contain a peptidase_C48 domain (FIG. 1f), a key feature of Ulp1 (ubiquitin-like-specific protease) proteases¹¹, which catalyze the maturation of small ubiquitin-like modifier (SUMO) propeptides and can play a role in regulating cell cycle progression in eukaryotes¹³. A number of bacteria and viruses are known to usurp SUMOylation pathways in the manipulation of their hosts^{14,15}. Type II WD0631 and WD0632 homologs are located within more complete phage haplotypes (FIG. 6), but the WD0632 homologs are truncated and lack recognized protein domains (FIG. 1f). Notably, all Wolbachia strains that contain type II homologs invariably contain at least one other copy of the operon that is type I and intact. Type III WD0631 homologs possess a cytochrome C552 domain involved in nitrate reduction, while type III WD0632 homologs contain a domain of unknown function (DUF1703) and a transmembrane domain (FIG. 1f). The functions of these domains are less well understood, but DUF1703 likely possesses nuclease activity¹⁶ and was previously found in a selfish genetic element that mediates embryonic lethality in Tribolium beetles¹⁷.

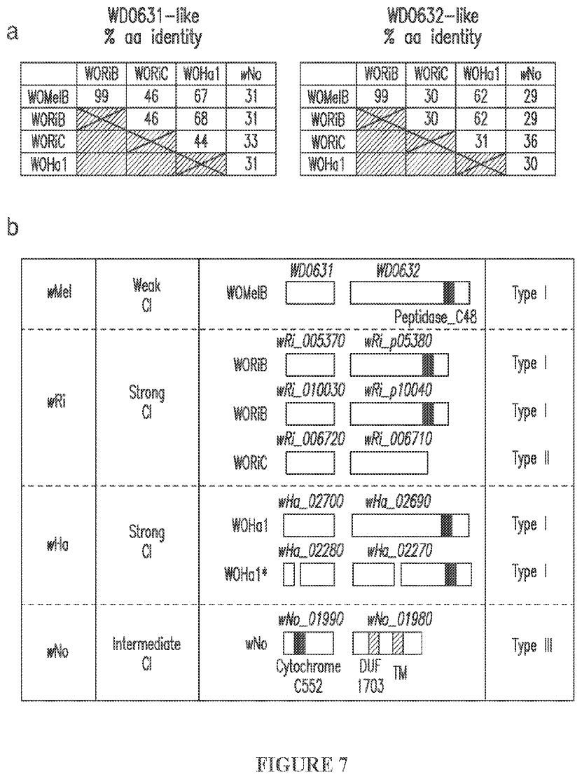

Consistent with these genes' role in CI, the degree of relatedness and presence or absence of shared operons of WD0631 and WD0632 between Wolbachia strains correlates with known patterns of bidirectional incompatibility (FIG. 1d). Among the strains wRi, wHa, and wNo, only wRi is able to rescue wMel-induced CI^{18,19}. We postulate that this is due to the fact that wRi and wMel share a highly related type I operon (99% amino acid identity), and thus likely also have a shared rescue factor, while wRi has an additional type II operon that may explain its ability to induce CI against wMel. Meanwhile, wHa has at most a 67% identity in the amino acid sequence of these proteins when compared to wMel, while wNo contains a type II operon that is only 31% identical (FIG. 7a). Additionally, the strength of CI varies considerably between different Wolbachia strains, and the relative degree of offspring lethality correlates with the number of copies of the WD0631/WD0632 operon that are present in each strain (FIG. 7b). Those strains with only one copy, such as wMel, have a comparatively weak CI phenotype, while those with two or three copies of the operon, such as wRi and wHa, cause strong CI¹⁹.

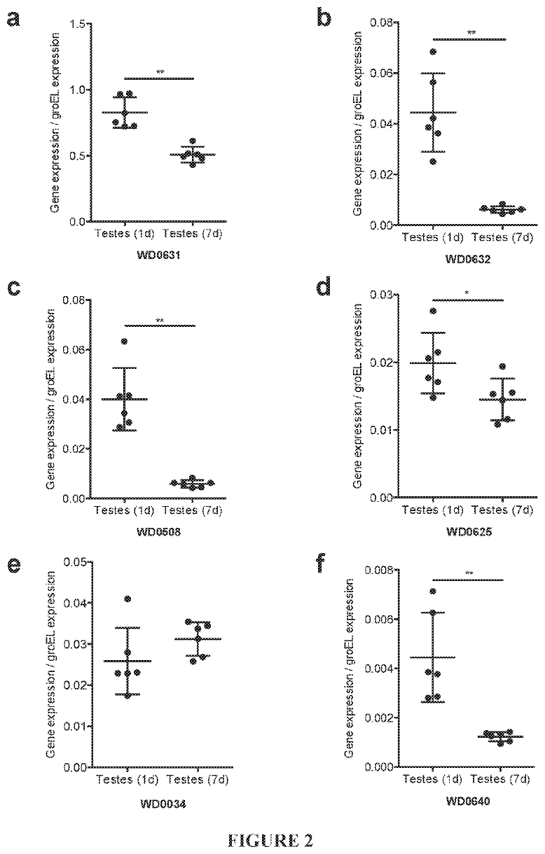

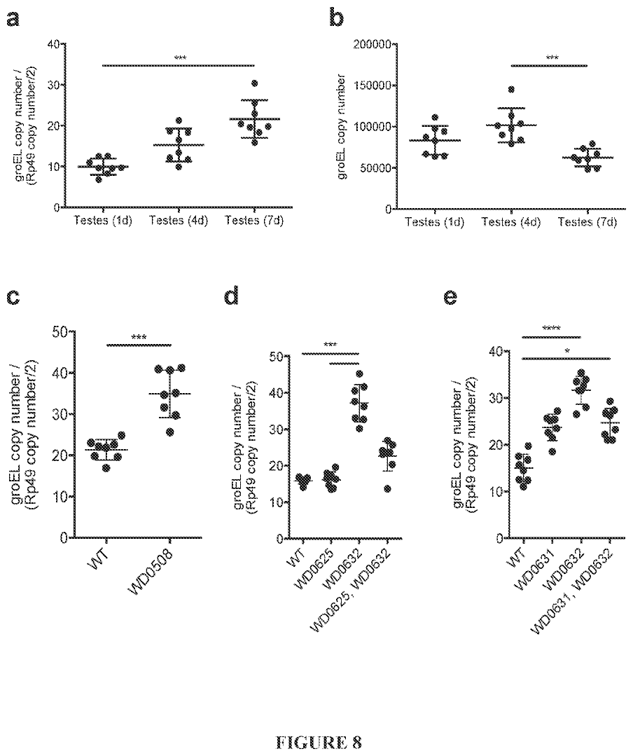

Given the many lines of evidence in support of these two genes, we next examined WD0631 and WD0632 for their functional role in CI, as well as control wMel genes that were not correlated with CI. These control genes are WD0034, which encodes a PAZ (Piwi, Argonaut, and Zwille) domain containing protein, and two prophage WO genes—WD0508, which encodes a putative transcriptional regulator, and WD0625, which encodes a DUF2466 domain likely acting as a nuclease or regulatory protein. We first examined the expression of CI effector candidates in the testes of wMel-infected, one-day-old and seven-day-old D. melanogaster males. Since the magnitude of CI is known to decrease dramatically between newly emerged and one-week-old males²⁰, we predicted that a CI effector would be expressed at a lower level in older male testes. Indeed, while WD0631 and WD0632 are expressed at different levels, both show a significantly lower transcription level in older versus younger males (FIG. 2a,b), as measured relative to the Wolbachia housekeeping gene groEL. Both phage-encoded control genes, WD0508 and WD0625, also exhibited this pattern, but the non-phage gene WD0034, did not (FIG. 2c-e). WD0640, which encodes phage WO structural protein gpW, was also reduced in older males, suggesting that phage genes in general are relatively downregulated in seven-day-old testes (FIG. 2f). The phenomenon of decreased CI in older males is not due to decreases in Wolbachia titer over time, as the copy number of Wolbachia groEL relative to D. melanogaster Rp49 increases as males age, and there is no significant difference in the absolute Wolbachia gene copies between one-day-old and seven-day-old males (FIG. 8a,8b).

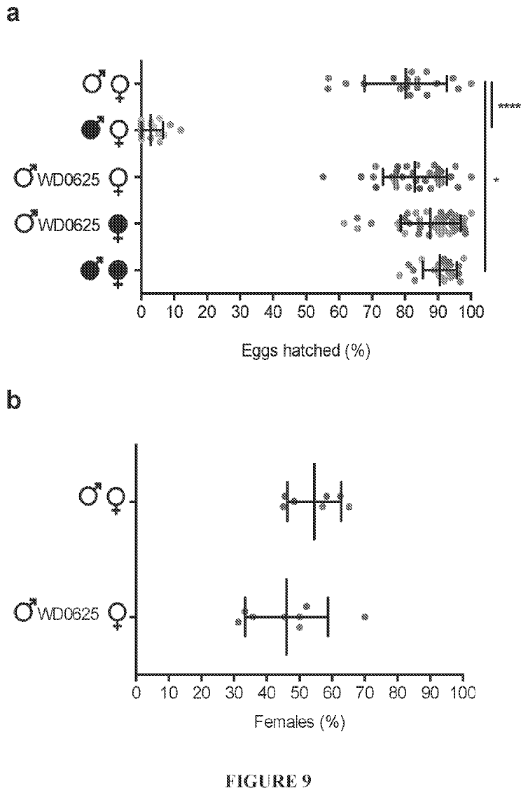

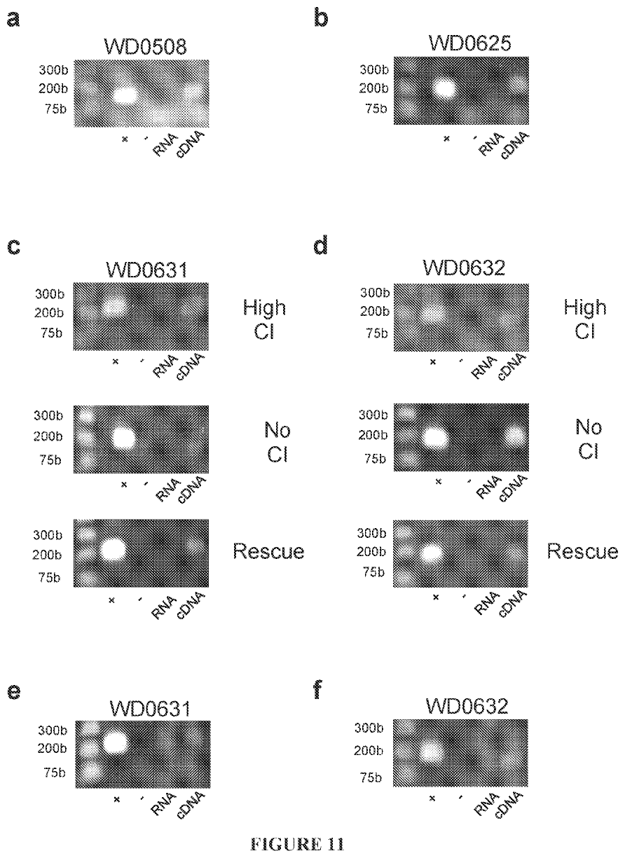

To directly test the function of these genes in CI, we generated transgenic D. melanogaster that express the candidate genes alone under the direction of an upstream activating sequence (UAS), since Wolbachia itself cannot be genetically transformed. We utilized a nanos-Gal4 driver line for tissue-specific expression predominantly in the germline^{21,22}. CI was determined by measuring the percentage of embryos that hatched into larvae. While wild type (WT) CI between infected males (less than one day old) and uninfected females led to significantly reduced hatch rates, transgene-expressing, uninfected males with each of the four candidate genes did not affect hatch rates when crossed to uninfected females (FIG. 3a, FIG. 9a). In addition, none of the four genes had an effect on sex ratios (FIG. 9b, 6). There are no phenotypic effects despite confirmed expression of each transgene in the testes (FIG. 11a-d).

As WD0631 and WD0632 are adjacent genes natively expressed as an operon¹¹, we reasoned that dual transgene expression of WD0631 and WD0632 in males may be required to induce CI. Indeed, dual expression significantly reduced hatch rates (74.2±18.5%) in comparison to that of uninfected males (96.2±2.5%) when mated to uninfected females (FIG. 3b). While this level of CI is incomplete, several crosses with transgenic males yielded hatch rates at levels comparable to the median hatch rate of WT CI (39.8±24.2%). It is possible that full induction of CI requires other factors or that our transgenic system does not express the genes at the ideal time, place, or amount to induce complete CI, though the genes do have confirmed expression in adult testes (FIG. 11c,d). Importantly, the observed defects are fully rescued by wMel-infected females (FIG. 3b), indicating that these genes are bona fide Wolbachia-induced CI genes rather than genes that artificially reduce hatch rates through off target effects. We provisionally name them here cytoplasmic incompatibility factors, cifA and cifB, for WD0631 and WD0632, respectively.

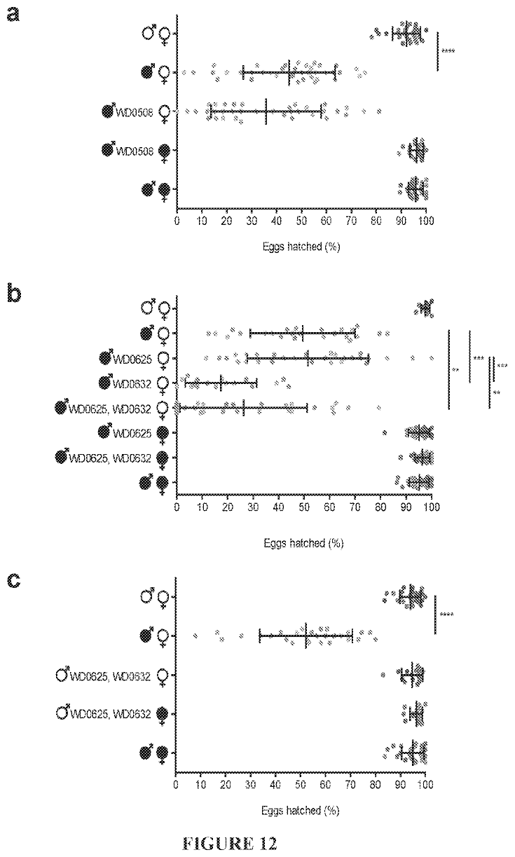

To test if the genes enhance WT CI levels that are naturally incomplete in D. melanogaster, we expressed WD0631 or WD0632 separately in wMel-infected male flies and found that hatch rates decreased significantly compared to WT CI crosses (FIG. 3c). In this context, we reason that both genes are adding to the quantity of CI effector molecules in wMel-infected tissues. This effect is not seen when control genes are expressed in wMel-infected males (FIG. 12a,b). Moreover, dual expression of the genes in wMel-infected flies reduces hatch rates still further than either gene alone, yet remains fully rescuable by wMel-infected females (FIG. 3c). Adding WD0625 to WD0632 in wMel-infected males does not increase CI beyond WD0632 alone (FIG. 12b), and the combination of WD0625 and WD0632 in uninfected males has no effect on hatching (FIG. 12c), indicating that the combination of WD0631 and WD0632 is uniquely required for induction of CI and that these findings are not an artifact of the transgenic system.

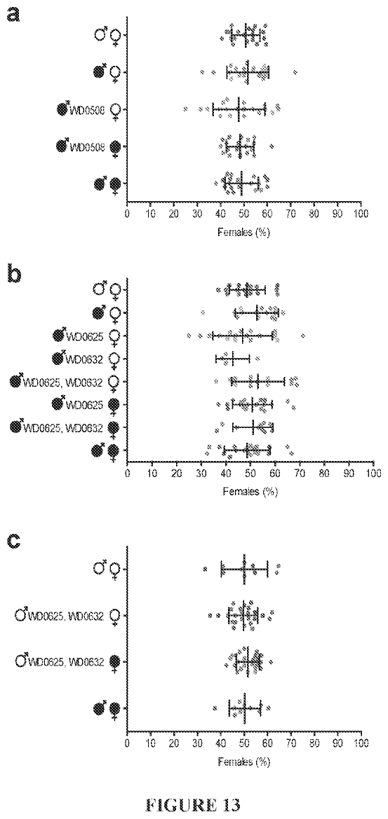

To rule out the possibility that enhancement of CI in the infected transgenic lines is due to an increase in Wolbachia titers, we monitored symbiont densities by measuring amplicons of single copy genes from Wolbachia and D. melanogaster. Although there were some differences in Wolbachia titers between the infected transgenic lines (FIG. 12c-e), these differences did not correlate with changes in the magnitude of CI, suggesting that decreased offspring viability was due to the direct effect of the transgenes rather than increased Wolbachia proliferation. Most notably, densities are significantly increased in control transgene WD0508 lines (FIG. 12c), but there is no effect on CI (FIG. 3a). Finally, none of these gene combinations had any effect on the sex ratios of offspring (FIG. 13).

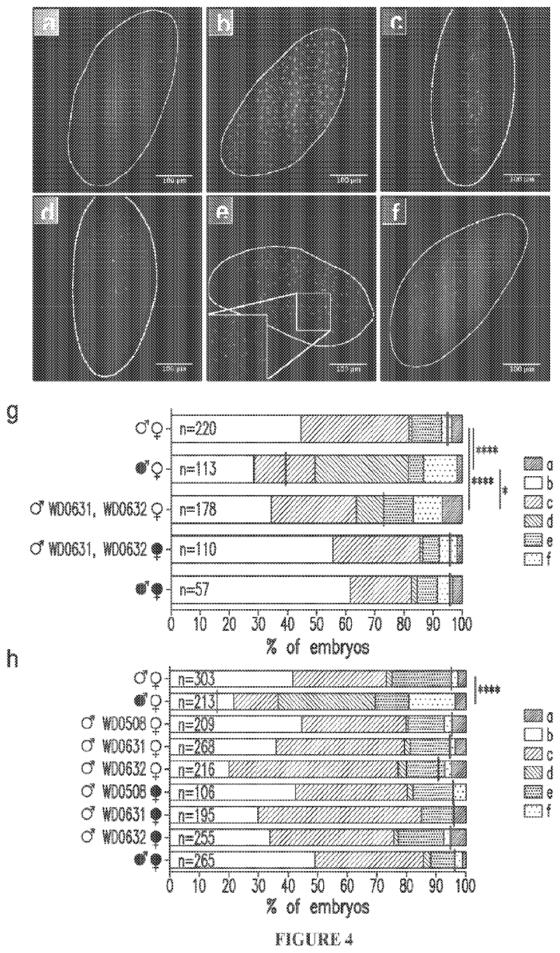

Next, we determined the similarity between the cytological defects observed during embryonic development in Wolbachia-induced CI versus CI from dual WD0631/WD0632 expressing transgenic flies. Although CI is classically recognized to cause failure of the first mitotic division^{23,24}, nearly half of the embryonic arrest in incompatible crosses occurs during advanced developmental stages in Drosophila simulans²⁵, a result that was first reported in Aedes polinesiensis mosquitoes²⁶. We examined embryos resulting from uninfected, wMel-induced CI, and transgenic crosses after one to two hours of development and binned their cytology into one of six phenotypes. While a few embryos in each cross were unfertilized (FIG. 4a), most embryos in WT crosses were either in normal late-stage preblastoderm (FIG. 4b), or in the syncytial blastoderm stage (FIG. 4c)²⁷. In the CI induced by wMel, embryos had one of three defects: arrest of cellular division after two to three mitotic divisions (FIG. 4d), arrest throughout development associated with moderate to extensive chromatin bridging as is classically associated with strong CI in D. simulans²⁴ (FIG. 4e), or arrest associated with regional failure of division in one segment of the embryo (FIG. 4f). After blindly scoring the number of embryos demonstrating each phenotype, we determined that arrest phenotypes d, e, and f were significantly more common in the offspring of dual WD0631/WD0632 transgenic males mated to uninfected females, but that these abnormalities were rescued in embryos from wMel-infected females (FIG. 4g). These effects were not seen with control gene WD0508 or with singular expression of WD0631 or WD0632 (FIG. 4h). These data again validate that Wolbachia-induced CI is recapitulated in dual WD0631/WD0632 transgenic flies.

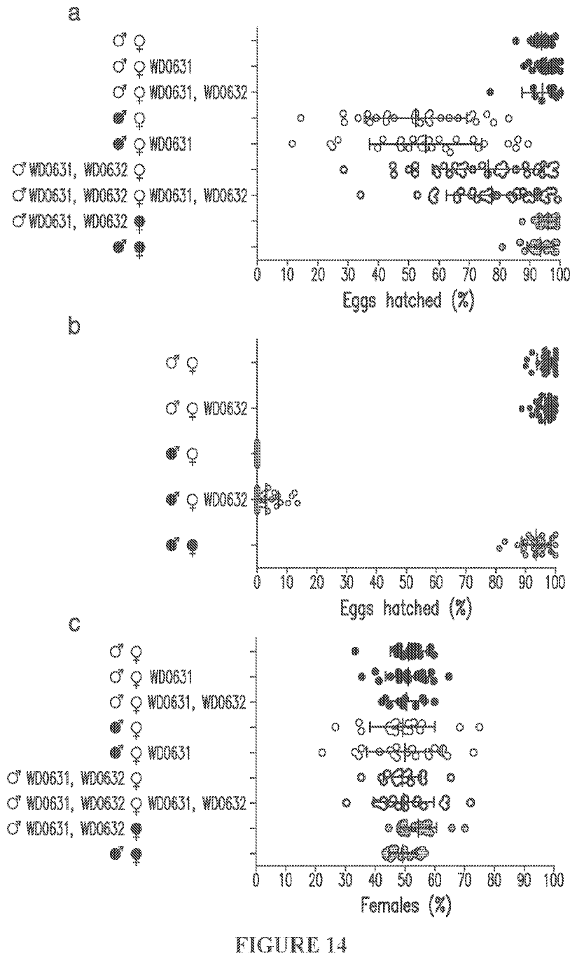

Finally, we evaluated whether WD0631 and WD0632 can rescue CI. Neither WD0631 nor WD0632, whether alone or combined, had an effect on hatch rates when expressed in uninfected females (FIG. 14a,b). WD0631- or WD0632-expressing females could not rescue wMel-induced CI, nor could WD0631/WD0632 dual-expressing females rescue CI induced by dual transgenic males (FIG. 14a,b), despite confirmed expression in ovaries (FIG. 11e,f). Transgene expression also had no effect on sex ratios (FIG. 14c). These data suggest that different genes underlie CI and rescue.

This study identifies, for the first time, genes that are responsible for inducing CI. While protein domain predictions suggest that the mechanism may involve nuclease or ubiquitin-modifying activity, the molecular basis of CI is further elucidated in a companion publication by Beckmann, et al, co-submitted with this manuscript. The discovery of CI effector genes is the first inroad to solving the genetic basis of reproductive parasitism, a phenomenon induced worldwide in an estimated hundreds of thousands to millions of arthropod species²⁸. The genes also have major implications for studying microbe-assisted speciation, because these genes likely underlie the CI-induced hybrid lethality observed between closely related species of Nasonia and Drosophila^{29,30}. Finally, these genes are important for arthropod pest or vector control strategies, as they could potentially be used as an alternative or adjunctive strategy to current Wolbachia-based paradigms aimed at controlling agricultural pests or curbing arthropod-borne transmission of infectious diseases⁵⁻⁸.

Methods

Comparative Genomics and Transcriptomics

MicroScope³¹ was used to select the set of genes comprising the core genomes of CI-inducing Wolbachia strains wMel [NC_002978.6]³², wRi [NC_012416.1]³³, wPip (Pel) [NC_010981.1]³⁴, and the recently sequenced wRec [RefSeq 1449268]⁹, while excluding the pan-genome of the mutualistic strain wBm [NC_006833.1]³⁵, using cutoffs of 50% amino acid identity and 80% alignment coverage. wAu microarray data were obtained from the original authors¹⁰ and genes that were present in CI-inducing strains wRi and wSim but absent or divergent in the non-CI strain wAu were selected.

For ovarian transcriptomics, one-day old females from wVitA infected-Nasonia vitripennis 12.1 were hosted as virgins on Sarcophaga bullata pupae for 48 hours to stimulate feeding and oogenesis. Females were then dissected in RNase-free 1X PBS buffer, and their ovaries were immediately transferred to RNase-free Eppendorf tubes in liquid nitrogen. Fifty ovaries were pooled for each of three biological replicates. Ovaries were manually homogenized with RNase-free pestles, and their RNA was extracted using the RNeasy Mini Kit (Qiagen) according to the manufacturer's protocol for purification of total RNA from animal tissues. After RNA purification, samples were treated with RQ1 RNase-free DNase (Promega), and ethanol precipitation was performed. PCR of RNA samples with Nasonia primers NvS6KQTF4 and NVS6KQTR4³⁶ confirmed that all samples were free of DNA contamination. RNA concentrations were measured with a Qubit 2.0 Fluorometer (Life Technologies) using the RNA HS Assay kit (Life Technologies), and approximately 5 μg of total RNA from each sample was used as input for the MICROBEnrich Kit (Ambion) in order to enrich for Wolbachia RNA in the samples. Microbially-enriched RNA was then ethanol-precipitated, and rRNA was depleted from the samples using the Ribo-Zero Magnetic kit (Illumina) according to manufacturer's protocol. Approximately 1.5 μg of microbially-enriched, rRNA-depleted RNA for each replicate was shipped to the University of Rochester Genomics Research Center for sequencing. Library preparation was performed using the Illumina ScriptSeq v2 RNA-Seq Library Preparation kit, and all samples were run multiplexed on a single lane of the Illumina HiSeq2500 (single-end, 100 bp reads). Raw reads were trimmed and mapped to the wVitA genome (PRJDB1504) in CLC Genomics Workbench 8.5.1 using a minimum length fraction of 0.9, a minimum similarity fraction of 0.8, and allowing one gene hit per read. With all three replicates combined, a total of 364,765 reads out of 41,894,651 (0.87%) mapped to the wVitA genome with the remaining reads mapping to the N. vitripennis host genome (GCF_000002325.3). All Wolbachia genes with greater than or equal to five RNA-seq reads, with the exception of the 16S and 23S RNA genes, were selected. For non-wMel data sets, the closest homologs in wMel were found using blastp in Geneious Pro v5.5.6³⁷.

### Protein Extraction and Mass Spectrometry

Protein was extracted from Culex pipiens tissues as described previously¹¹. Ovaries from 30 wPip (Buckeye)-infected mosquitoes were dissected in 100% ethanol and collected in a 1.5 ml tube filled with 100% ethanol. Pooled tissues were sonicated at 40 mA for 10 seconds in a Kontes GE 70.1 ultrasonic processor, and trichloroacetic acid (TCA) was added to a final concentration of 10% (v/v). After centrifugation at 13,000 rpm in a microcentrifuge, pellets were washed with acetone:water (9:1), dried, and stored at −20° C. Samples were directly submitted to the University of Minnesota's Center for Mass Spectrometry and Proteomics for iTRAQ (isobaric tagging for relative and absolute quantification) analysis. Proteins were sorted according to their relative abundance as determined by the number of spectra from the single most abundant peptide. Because proteins can often produce varying amounts of detectable tryptic peptides depending upon protein size and lysine/arginine content, we counted only the single most abundant peptide for each protein. This quantification is justified by previous reports¹¹ showing that the two most abundant proteins are the Wolbachia surface protein (WSP; gil190571332) and another putative membrane protein (gil190570988). Only proteins with at least three unique peptides (95% confidence) detected were reported, and using this criterion the false discovery rate was zero.

### Gene Expression Assays

Expression of CI candidates was tested with RT-qPCR on pools of 20 pairs of testes from one-day-old and seven-day-old virgin males. RNA was extracted with the Qiagen RNeasy mini kit, DNase treated with TURBO DNase (Life Technologies) and cDNA was generated with Superscript III Reverse Transcriptase (Invitrogen). Delta delta Ct analysis against the housekeeping gene groEL was used to determine relative gene expression.

### Evolutionary Analyses

WD0631 and WD0632 were used as queries to perform a BLASTp search of NCBI's nonredundant (nr) protein sequence database with algorithm parameters based on a word-size of six and BLOSUM62 scoring matrix³⁸. Homologs were selected based on the satisfaction of three criteria: (i) E-value≤10⁻²⁰, (ii) query coverage greater than 60%, and (iii) presence in fully sequenced Wolbachia and/or phage WO genomes. FtsZ and gpW proteins were identified for all representative Wolbachia and phage WO genomes, respectively. Protein alignments were performed using the MUSCLE plugin³⁹ in Geneious Pro v8.1.7³⁷; the best models of selection, according to the corrected Akaike Information Criteria (AICc)⁴⁰, were estimated using the ProtTest server⁴¹; and phylogenetic trees were built using the MrBayes plugin⁴² in Geneious. Putative functional domains were identified using NCBI's BLASTP, Wellcome Trust Sanger Institute's PFAM database⁴³ and EMBL's Simple Modular Architecture Research Tool (SMART)⁴⁴.

Fly Rearing

D. melanogaster were reared on standard cornmeal and molasses based media. Stocks were maintained at 25C while virgin flies were stored at room temperature. During virgin collections, stocks were kept at 18C overnight and 25C during the day. Wolbachia uninfected lines were generated through tetracycline treatment for three generations. Briefly, tetracycline was dissolved in ethanol and then diluted in water to a final concentration of 1 mg/mL. 1 mL of this solution was added to 50 mL of media (final concentration of 20 μg/mL). Freshly treated media was used for each generation. Infection status was confirmed with PCR using Wolb_F and Wolb_R3 primers⁴⁵, and flies were reared on untreated media for at least three additional generations before being utilized.

Transgenic Flies

Each CI candidate gene was cloned into the pTIGER plasmid for transformation and expression in D. melanogaster⁴⁶. pTIGER was designed for targeted integration into the D. melanogaster genome using PhiC31 integrase⁴⁷ and tissue-specific, inducible expression through the Gal4-UAS system⁴⁸. Cloning was performed using standard molecular biology techniques and plasmids were purified and sequence-confirmed before injection. At least 200 D. melanogaster embryos were injected per gene by Best Gene, Inc (Chino Hills, CA), and transformants were selected based on w+eye color. Isogenic, homozygous lines were maintained when possible, or isogenic heterozygous flies were maintained when homozygous transgenics were inviable (WD0625/CyO). WD0508 and WD0631 insertion was carried out with the y¹ M{vas-int. Dm}ZH-2A w *; P{CaryP}attP40 line. WD0625 was inserted into BSC9723 with the genotype: y¹ M{vas-int. Dm}ZH-2A w *; PBac{y+-attP-3B} VK00002. WD0632 insertion was done using BSC8622 with the genotype: y¹ w^{67c23}; P{CaryP}attP2.

Wolbachia titers

For FIG. 8c-e, brothers of those used in the corresponding hatch rates were utilized. Testes were dissected from males in cold PBS. Pools of testes from 15 males were used for each sample, and DNA was extracted using the Gentra Puregene Tissue kit (Qiagen). Quantitative PCR was performed on a Bio-Rad CFX-96 Real-Time System using iTaq Universal SYBR Green Supermix (Bio-Rad). Absolute quantification was achieved by comparing all experimental samples to a standard curve generated on the same plate. The Rp49 standard template was generated using the same primers as those used to determine quantity while the groEL standard template was generated using groELstd_F and groELstd_R primers that we designed. qPCR conditions: 50C 10 min, 95C 5 min, 40×(95C 10 sec, 55C 30 sec), 95C 30 sec. Followed by melt curve analysis (0.5C steps from 65-95C for 5 sec each). To obtain a more accurate Wolbachia:host cell ratio, it was assumed that each host cell has two copies of Rp49 and each Wolbachia cell has one copy of groEL.

### Hatch Rate Assays

Parental females, unless expressing a transgene, were WT y¹w* flies (wMel-infected or uninfected) and aged for 2-5 days before crossing. Parental males were created by crossing nanos-Gal4 virgin females (wMel-infected or uninfected) with either WT or UAS-candidate gene-transgenic males. Only the first males emerging from these crosses were used to control for the older-brother effect associated with CI²⁰. In assays to determine whether CI was increased, virgin males were aged for 3-4 days before crossing to reduce the level of WT CI. In these experiments, care was taken to match the age of males between experimental and control crosses. In all other assays, virgin males were used within 30 hours of emergence. 32-64 individual crosses were used for each crossing condition. To perform the hatch rate assays, a single male and single female were placed in an 8 oz, round bottom, polypropylene Drosophila stock bottle. A grape juice-agar plate with a small amount of yeast mix (1 part water: 2 parts dry yeast) smeared on top was placed in the bottle opening and affixed with tape. Grape juice-agar plates consist of the lids from 35×10 mm culture dishes (CytoOne). 12.5 g of agar is mixed in 350 mL of ddH2O and autoclaved. In a separate flask, 10 mL of ethanol is used to dissolve 0.25 g tegosept (methyl 4-hyrdoxybenzoate). 150 mL of Welch's grape juice is added to the tegosept mix, combined with the agar, and poured into plates.

Hatch rate bottles were placed in a 25C incubator overnight (˜16 hours). After this initial incubation the grape plates were discarded and replaced with freshly yeasted plates. After an additional 24 hours the adult flies were then removed and frozen for expression analysis and the embryos on each plate were counted. These plates were then incubated at 25C for 36 hours before the number of unhatched embryos was counted. Larvae were moved from these plates and placed in vials of fly media with one vial being used for each individual grape plate to be assayed for sex ratios at adulthood. A total of 10-20 vials were used for each cross type. Any crosses with fewer than 25 embryos laid were discarded from the hatching analysis while vials with fewer than 10 adults emerging were discarded from the sex ratio analysis. Statistical analysis and outlier removal, utilizing the ROUT method, were performed using Graphpad Prism v6 software.

### Transgene RT-PCR

Pools of six pairs of testes or ovaries were dissected from parents utilized in hatch rate assays. In samples designated “High CI” and “No CI”, the males correspond to crosses that had low or normal hatch rates, respectively. For all other samples the flies utilized were chosen at random. RNA was extracted using the Direct-zol RNA MiniPrep Kit (Zymo), DNase treated with DNA-free (Ambion, Life Technologies) and cDNA was generated with SuperScript VILO (Invitrogen). 30 cycles of PCR were performed against positive controls (extracted DNA), negative controls (water), RNA, and cDNA with the following conditions: 95C 2 min, 30×(95C 15 sec, 56C 30 sec, 72C 30 sec), 72C 5 min.

### Embryo Imaging

Embryos were collected in a fashion similar to hatch rate assays except bottles contained 60-80 females and 15-20 males. After an initial 16 hours of mating, fresh grape plates were added and embryos were removed after 60 minutes. The embryo-covered plates were then placed in the incubator at 25C for a further 60 minutes to ensure each embryo was at least 1 hour old. Embryos were then moved to a small mesh basket and dechorionated in 50% bleach for 1-3 minutes. These were then washed in embryo wash solution (7% NaCl, 0.5% Triton X-100) and moved to a small vial with ˜2 mL heptane. An equal amount of methanol was added to the vial and then vigorously shaken for 15 seconds. The upper heptane layer, and most of the methanol, was then removed and the embryos moved to fresh methanol in a 1.5 mL microcentrifuge tube. Embryos were stored overnight at 4° C. for clearing. The old methanol was then removed and replaced with 250 μL of fresh methanol along with 750 μL of PBTA (1x PBS, 1% BSA, 0.05% Triton X-100, 0.02% sodium azide). After inverting the tube several times, the solution was removed and replaced with 500 μL PBTA. Embryos were then rehydrated for 15 minutes on a rotator at room temperature. After rehydrating, the PBTA was replaced with 100 μL of a 10 mg/mL RNase solution and incubated at 37ºC for 2 hours. The RNase was then removed and embryos were washed several times with PBS followed by a final wash with PBS-Azide (1x PBS, 0.02% sodium azide). After removing the PBS-Azide, embryos were mounted on glass slides with ProLong Diamond Antifade (Life Technologies) spiked with propidium iodide (Sigma-Aldrich) to a final concentration of 1 ug/mL. Imaging was performed at the Vanderbilt Cell Imaging Shared Resource using a Zeiss LSM 510 META inverted confocal microscope. All scores were performed blind and image analysis was done using ImageJ software49.

### REFERENCES CITED IN EXAMPLE 1

1 Yen, J. H. & Barr, A. R. New hypothesis of the cause of cytoplasmic incompatibility in Culex pipiens L. Nature 232, 657-658 (1971).
2 Kent, B. N. & Bordenstein, S. R. Phage WO of Wolbachia: lambda of the endosymbiont world. Trends in microbiology 18, 173-181 (2010).
3 Brucker, R. M. & Bordenstein, S. R. Speciation by symbiosis. Trends in ecology & evolution 27, 443-451 (2012).
4 Shropshire, J. D. & Bordenstein, S. R. Speciation by Symbiosis: the Microbiome and Behavior. MBio 7 (2016).
5 Zabalou, S. et al. Wolbachia-induced cytoplasmic incompatibility as a means for insect pest population control. Proceedings of the National Academy of Sciences of the United States of America 101, 15042-15045 (2004).
6 O'Connor, L. et al. Open release of male mosquitoes infected with a wolbachia biopesticide: field performance and infection containment. PLOS Negl Trop Dis 6, e1797 (2012).
7 Walker, T. et al. The wMel Wolbachia strain blocks dengue and invades caged Aedes aegypti populations. Nature 476, 450-453 (2011).
8 Dutra, Heverton Leandro C. et al. Wolbachia Blocks Currently Circulating Zika Virus Isolates in Brazilian Aedes aegypti Mosquitoes. Cell Host & Microbe, doi: 10.1016/j.chom.2016.04.021.
9 Metcalf, J. A., Jo, M., Bordenstein, S. R., Jaenike, J. & Bordenstein, S. R. Recent genome reduction of Wolbachia in Drosophila recens targets phage WO and narrows candidates for reproductive parasitism. PeerJ 2, e529 (2014).
10 Ishmael, N. et al. Extensive genomic diversity of closely related Wolbachia strains. Microbiology 155, 2211-2222 (2009).
11 Beckmann, J. F. & Fallon, A. M. Detection of the Wolbachia protein WPIP0282 in mosquito spermathecae: implications for cytoplasmic incompatibility. Insect biochemistry and molecular biology 43, 867-878 (2013).
12 Bordenstein, S. R. & Bordenstein, S. R. Novel eukaryotic association module in phage WO genomes from Wolbachia. Nature Communications (2016).
13 Li, S. J. & Hochstrasser, M. A new protease required for cell-cycle progression in yeast. Nature 398, 246-251 (1999).
14 Wimmer, P. & Schreiner, S. Viral Mimicry to Usurp Ubiquitin and SUMO Host Pathways. Viruses 7, 4854-4872 (2015).
15 Wimmer, P., Schreiner, S. & Dobner, T. Human pathogens and the host cell SUMOylation system. Journal of virology 86, 642-654 (2012).
16 Knizewski, L., Kinch, L. N., Grishin, N. V., Rychlewski, L. & Ginalski, K. Realm of PD-(D/E)XK nuclease superfamily revisited: detection of novel families with modified transitive meta profile searches. BMC Struct Biol 7, 40 (2007).
17 Lorenzen, M. D. et al. The maternal-effect, selfish genetic element Medea is associated with a composite Tc1 transposon. Proceedings of the National Academy of Sciences of the United States of America 105, 10085-10089 (2008).
18 Zabalou, S. et al. Multiple rescue factors within a Wolbachia strain. Genetics 178, 2145-2160 (2008).
19 Poinsot, D., Bourtzis, K., Markakis, G., Savakis, C. & Mercot, H. Wolbachia transfer from Drosophila melanogaster into D. simulans: Host effect and cytoplasmic incompatibility relationships. Genetics 150, 227-237 (1998).
20 Yamada, R., Floate, K. D., Riegler, M. & O'Neill, S. L. Male development time influences the strength of Wolbachia-induced cytoplasmic incompatibility expression in Drosophila melanogaster. Genetics 177, 801-808 (2007).
21 Rorth, P. Gal4 in the Drosophila female germline. Mechanisms of development 78, 113-118 (1998).
22 White-Cooper, H. Tissue, cell type and stage-specific ectopic gene expression and RNAi induction in the Drosophila testis. Spermatogenesis 2, 11-22 (2012).
23 Serbus, L. R., Casper-Lindley, C., Landmann, F. & Sullivan, W. The genetics and cell biology of Wolbachia-host interactions. Annual review of genetics 42, 683-707 (2008).
24 Landmann, F., Orsi, G. A., Loppin, B. & Sullivan, W. Wolbachia-mediated cytoplasmic incompatibility is associated with impaired histone deposition in the male pronucleus. PLOS pathogens 5, e1000343 (2009).
25 Callaini, G. & Riparbelli, M. G. Fertilization in Drosophila melanogaster: centrosome inheritance and organization of the first mitotic spindle. Dev Biol 176, 199-208 (1996).
26 Wright, J. D. & Barr, A. R. Wolbachia and the normal and incompatible eggs of Aedes polynesiensis (Diptera: Culicidae). Journal of invertebrate pathology 38, 409-418 (1981).
27 Bate, M. & Arias, A. M. The Development of Drosophila Melanogaster. (Cold Spring Harbor Laboratory Press, 1993).
28 Zug, R. & Hammerstein, P. Still a host of hosts for wolbachia: analysis of recent data suggests that 40% of terrestrial arthropod species are infected. PloS one 7, e38544 (2012).
29 Jaenike, J., Dyer, K. A., Cornish, C. & Minhas, M. S. Asymmetrical reinforcement and Wolbachia infection in Drosophila. PLOS biology 4, e325 (2006).
30 Bordenstein, S. R., O'Hara, F. P. & Werren, J. H. Wolbachia-induced incompatibility precedes other hybrid incompatibilities in Nasonia. Nature 409, 707-710 (2001).
31 Vallenet, D. et al. MicroScope: a platform for microbial genome annotation and comparative genomics. Database: the journal of biological databases and curation 2009, bap021 (2009).
32 Wu, M. et al. Phylogenomics of the reproductive parasite Wolbachia pipientis wMel: a streamlined genome overrun by mobile genetic elements. PLOS biology 2, E69 (2004).
33 Klasson, L. et al. The mosaic genome structure of the Wolbachia wRi strain infecting Drosophila simulans. Proc Natl Acad Sci USA 106, 5725-5730 (2009).
34 Klasson, L. et al. Genome evolution of Wolbachia strain wPip from the Culex pipiens group. Mol Biol Evol 25, 1877-1887 (2008).
35 Foster, J. et al. The Wolbachia genome of Brugia malayi: endosymbiont evolution within a human pathogenic nematode. PLOS biology 3, e121 (2005).
36 Bordenstein, S. R. & Bordenstein, S. R. Temperature affects the tripartite interactions between bacteriophage WO, Wolbachia, and cytoplasmic incompatibility. PloS one 6, e29106 (2011).
37 Kearse, M. et al. Geneious Basic: an integrated and extendable desktop software platform for the organization and analysis of sequence data. Bioinformatics 28, 1647-1649 (2012).
38 Johnson, M. et al. NCBI BLAST: a better web interface. Nucleic acids research 36, W5-9 (2008).
39 Edgar, R. C. MUSCLE: multiple sequence alignment with high accuracy and high throughput. Nucleic acids research 32, 1792-1797 (2004).
40 Hurvich, C. M. & Tsai, C.- L. A Corrected Akaike Information Criterion for Vector Autoregressive Model Selection. Journal of Time Series Analysis 14, 271-279 (1993).
41 Abascal, F., Zardoya, R. & Posada, D. ProtTest: selection of best-fit models of protein evolution. Bioinformatics 21, 2104-2105 (2005).
42 Ronquist, F. et al. MrBayes 3.2: efficient Bayesian phylogenetic inference and model choice across a large model space. Systematic biology 61, 539-542 (2012).
43 Finn, R. D. et al. The Pfam protein families database: towards a more sustainable future. Nucleic acids research 44, D279-285 (2016).
44 Letunic, I., Doerks, T. & Bork, P. SMART 7: recent updates to the protein domain annotation resource. Nucleic acids research 40, D302-305 (2012).
45 Casiraghi, M., Anderson, T. J., Bandi, C., Bazzocchi, C. & Genchi, C. A phylogenetic analysis of filarial nematodes: comparison with the phylogeny of Wolbachia endosymbionts. Parasitology 122 Pt 1, 93-103 (2001).
46 Ferguson, S. B., Blundon, M. A., Klovstad, M. S. & Schupbach, T. Modulation of gurken translation by insulin and TOR signaling in Drosophila. Journal of cell science 125, 1407-1419 (2012).
47 Groth, A. C., Fish, M., Nusse, R. & Calos, M. P. Construction of transgenic Drosophila by using the site-specific integrase from phage phiC31. Genetics 166, 1775-1782 (2004).
48 Southall, T. D., Elliott, D. A. & Brand, A. H. The GAL4 System: A Versatile Toolkit for Gene Expression in Drosophila. CSH protocols 2008, pdb top49 (2008).
49 Schneider, C. A., Rasband, W. S. & Eliceiri, K. W. NIH Image to ImageJ: 25 years of image analysis. Nature methods 9, 671-675 (2012).
50 Vizcaino, J. A. et al. 2016 update of the PRIDE database and its related tools. Nucleic acids research 44, D447-456 (2016).
51 Bossan, B., Koehncke, A. & Hammerstein, P. A new model and method for understanding Wolbachia-induced cytoplasmic incompatibility. PloS one 6, e19757 (2011).

### Example 2. A Wolbachia Deubiquitylating Enzyme Induces Cytoplasmic Incompatibility

Wolbachia are obligate intracellular bacteria¹ that infect many arthropods, including nearly two-thirds of all insect species.² These symbionts often manipulate host reproduction to enhance their inheritance through the female germline. The most common reproductive alteration is cytoplasmic incompatibility (CI),³⁻⁵ wherein eggs from uninfected females fail to develop when fertilized by sperm from Wolbachia-infected males. By contrast, if female and male partners are both infected, the resulting embryos are viable. CI is a potent gene-drive mechanism that impacts population structure⁶ and speciation,⁷ but its molecular mechanism remained unknown. In this example, we show a Wolbachia deubiquitylating enzyme (DUB) contributes directly to CI. The CI-inducing DUB, CidB, is a cysteine protease encoded in a two-gene operon; the other protein, CidA, binds CidB. Binding affinity is highest between cognate partners of cidA-cidB-related operons from different Wolbachia strains. In transgenic fruit flies, the cidA-cidB operon mimics CI when sperm introduce the DUB into eggs; an operon with a catalytically inactive DUB does not induce sterility. Toxicity is recapitulated in yeast by CidB alone: DUB activity is required for toxicity but is rescued by coexpressed CidA, suggesting a toxin-antidote relationship between CidB and CidA. A related operon from the same Wolbachia strain, wPip, involves a putative nuclease (CinB) rather than a DUB; analogous binding, toxicity and rescue in yeast were observed. These results identify a primary mechanism for CI involving toxin and antidote-like proteins secreted into germline cells by resident bacteria and suggest potential new methods for control of insect pests and disease vectors, such as mosquitoes transmitting the Dengue fever and Zika viruses.

The mechanism of CI is frequently modeled as a toxin-antidote (modification-rescue) system in which sperm carry a Wolbachia toxin^{8,9} that can be conditionally rescued in the egg by a Wolbachia-encoded antidote (FIG. 15a, b).^{5,10-13} Normally, upon fertilization the sperm-derived pronucleus undergoes nuclear envelope breakdown and exchanges protamines for maternal histones.¹⁴ Subsequently, male and female pronuclei juxtapose (but do not fuse) and undergo DNA replication prior to the first zygotic mitosis. Chromosomes condense, align at metaphase, and separate in anaphase.¹⁵ In CI crosses, paternal chromatin fails to condense properly for the first cell cycle. This induces lethal missegregation and bridging of paternal DNA at anaphase.¹⁶⁻¹⁸

Wolbachia themselves are removed from sperm in the later stages of spermatid differentiation.¹¹ Therefore, in a previous proteomic search, we looked for Wolbachia (wPip strain) proteins associated with Wolbachia-modified mosquito sperm deposited in the spermathecae of female mosquitoes following mating. We identified the Wolbachia protein WPA0282.¹⁹ The wPa_0282 gene is part of a two-gene operon (FIG. 15c). Given our identification (see below) of the second gene product, WPA0283, as a CI-inducing DUB, we have renamed the genes cidA (wPa_0282) and cidB (wPa_0283). Ubiquitin is a small protein that posttranslationally modifies many proteins and has many functions.²⁰ Protein ubiquitylation is highly dynamic, and is reversed by many different cellular DUBs.²¹ Phenotypic evidence from diverse Wolbachia strains suggests that the toxin and antidote functionalities of CI arise from at least two independent genes.¹⁰ Moreover, most toxin-antidote systems studied in bacteria are organized as simple two-gene operon structures (5′-antidote-toxin-3′).^{22,23} Therefore, we hypothesized that the cidA-cidB operon products might be the executers of CI.

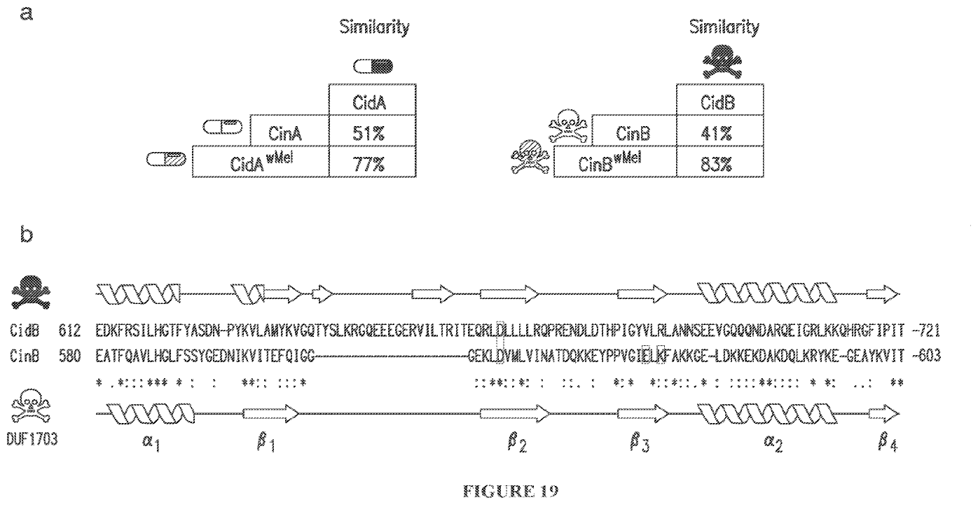

As Wolbachia strains diverge within a host species, they accumulate mutations in their corresponding CI systems and become bidirectionally incompatible,^{3,24} suggesting that their respective toxin-antidote genes have evolved mutually exclusive specificities.¹⁰ Interestingly, Wolbachia genomes from Culex pipiens mosquitoes show extensive genetic duplication and divergence of the putative toxin-antidote operon, potentially accounting for multiple incompatibilities. Wolbachia strain wPip, for example, has two related operons (FIG. 15c, d). The second operon encodes proteins related to CidA and CidB, but the putative toxin includes what appears to be a functional nuclease domain (DUF1703)²⁵ rather than a C48/Ulp1-like DUB motif (FIG. 15d); we have therefore renamed the two genes in this operon as cinA (wPa_0294) and cinB (wPa_0295). The putative toxins from both operons, CidB and CinB, appear to share a common nuclease ancestor (FIG. 19; FIG. 15c, dotted lines), but the apparent nuclease active-site residues are not maintained in CidB. Importantly, the predicted enzymatic competence of the toxin components of these operons correlates with ability to induce CI in diverse Wolbachia strains.

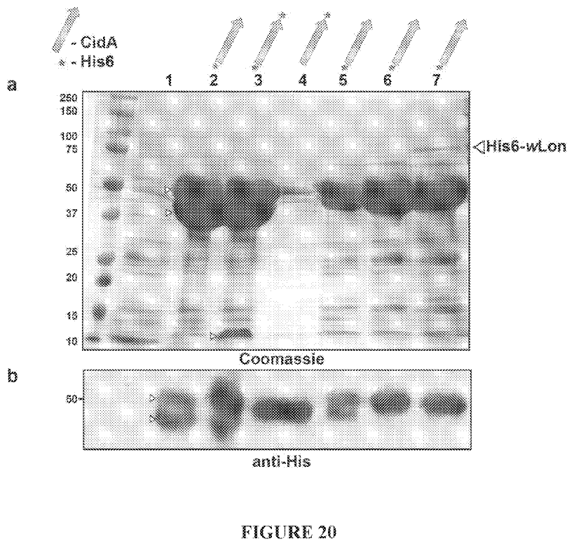

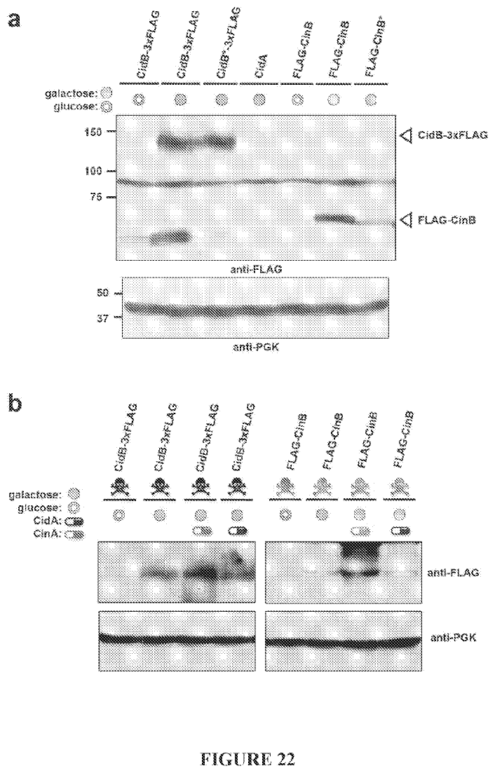

In many toxin-antidote systems, toxin and antidote bind one another.²³ We therefore expressed recombinant tagged constructs of the cidA-cidB operon proteins (FIG. 20, 21) and examined their interactions. Pulldown of His6-tagged CidA from extracts of E. coli expressing both His6-CidA and CidB brought down the CidB protein (FIG. 22a, b). We observed similar binding of the cognate partners His6-CinA and CinB (FIG. 22c, d). If differential affinity of operon-encoded toxins and antidotes accounts for bi-directional incompatibility, then the putative toxin-antidote pairs expressed from the same operon would be predicted to associate preferentially relative to their noncognate partners²⁶ from other operons. To test this, we purified His6-tagged copies of CidB, CinB, and CidB^{wMel} (the latter is from a Wolbachia strain isolated from Drosophila melanogaster). These putative toxin proteins were incubated with extracts of the corresponding FLAG-tagged antidote variants, and binding was assessed (FIG. 15f). Binding was indeed much stronger between the cognate proteins from each operon. These results are consistent with a model in which operon-specific differences in toxin-antidote binding underlie the bidirectional incompatibilities and partial rescues seen in genetic crosses with different Wolbachia strains.

When divergent CI-causing Wolbachia strains are introduced into different insects by microinjection, CI is recapitulated.²⁷⁻³⁰ This indicates that Wolbachia CI factors can operate in a broad range of hosts. To test the toxin-antidote model for CI in a heterologous eukaryotic host, we expressed the Cid and Cin proteins in the yeast Saccharomyces cerevisiae (FIG. 16). The putative toxins CidB and CinB both caused temperature-sensitive growth inhibition when introduced into yeast. Growth was rescued by coexpression of the cognate antidotes, CidA and CinA, respectively. When the predicted cysteine protease active site in CidB³¹ was mutated from Cys to Ala (CidB* in FIG. 16a), toxicity was lost. Similarly, upon mutation of the predicted nuclease catalytic residues in CinB (CinB*, FIG. 16a), temperature-sensitive lethality was no longer observed. Changes in toxin protein levels cannot account for the loss of toxicity, at least in the case of CidB* (FIG. 22). Importantly, only the cognate antidotes rescued growth when coexpressed with the toxins (FIG. 16b). Toxicity and rescue for both operons was seen in two different yeast backgrounds (BY4741 and W303a). These results show first, that the Wolbachia-encoded proteins can behave as toxin-antidote pairs in vivo; second, that toxicity depends on enzymatic activity (see below) of the CidB and CinB proteins; and finally, that suppression of toxicity in vivo correlates with protein binding preferences in vitro.

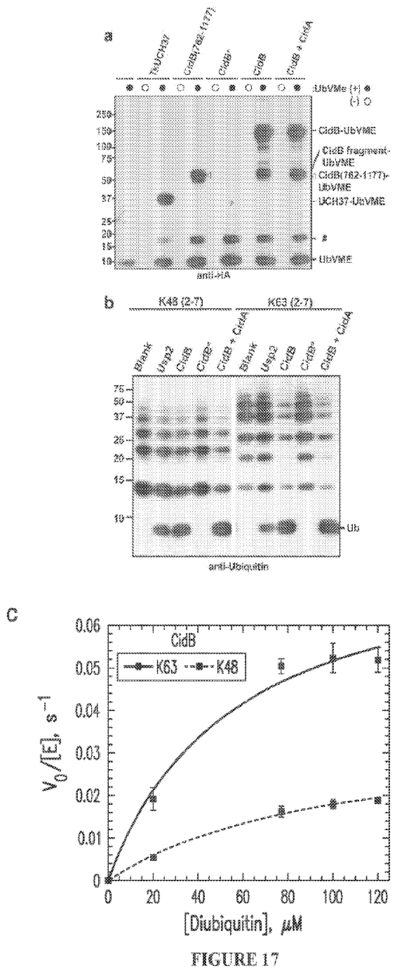

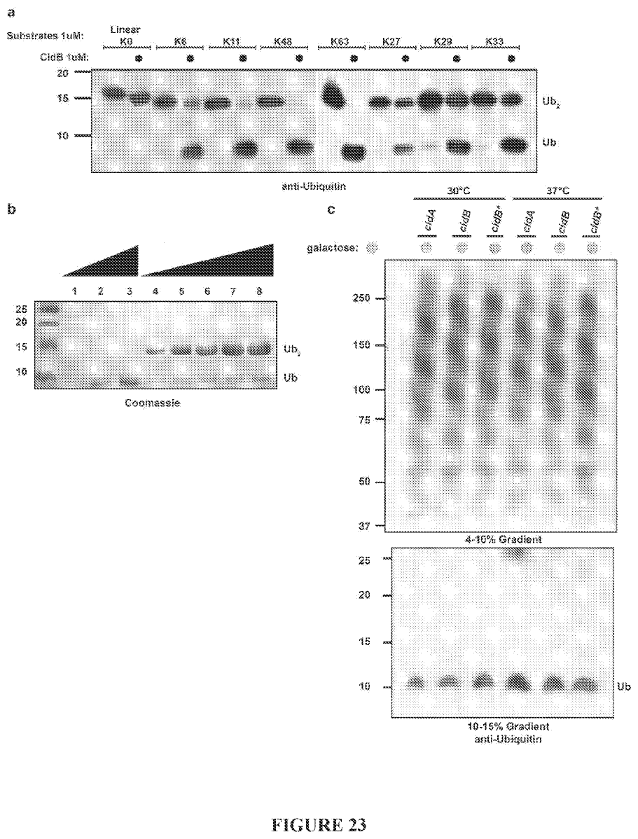

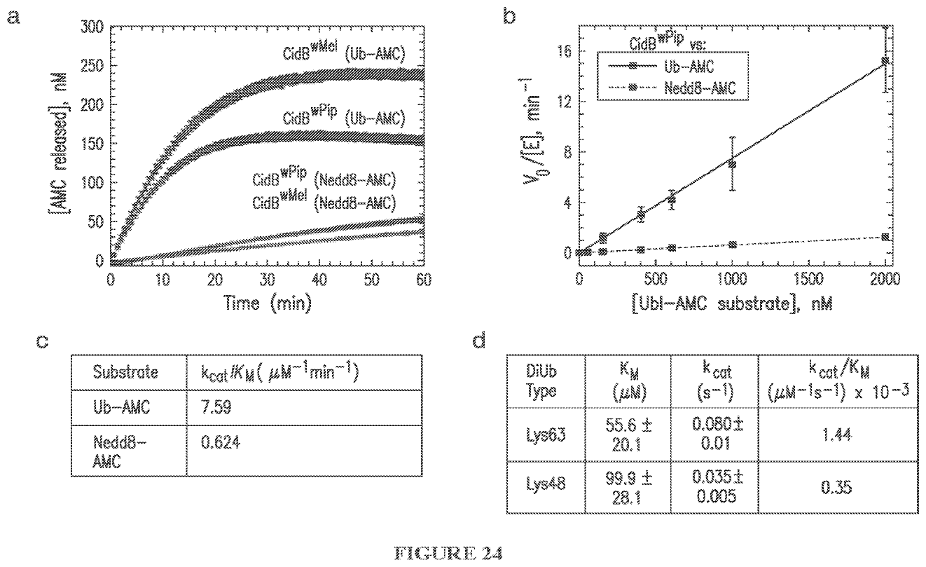

Next, we sought to characterize the enzymatic activity of CidB. We initially expected it to be a protease specific for the SUMO ubiquitin-like protein (UBL) since it bears a C48/Ulp1-like domain;³¹ however, the purified protein did not cleave fluorogenic SUMO-AMC or SUMO-peptide fusions (data not shown). By contrast, both full-length and a truncated C48/Ulp1-like domain-bearing version of CidB reacted with the suicide inhibitor HA-ubiquitin-vinyl methyl ester (HA-Ub-VME) (FIG. 17a). Enzyme activity was tested against polyubiquitin chains with isopeptide linkages (C-terminal ubiquitin carboxyl group linked to a ubiquitin lysine-amino group) involving either Lys48 or Ly63 residues or against ubiquitin dimers linked through each of the seven different ubiquitin lysines. CidB cleaved the isopeptide bonds in all of them and had a preference for Lys63 linkages in quantitative assays (FIG. 17b, c; FIG. 23). The enzyme did not cleave Met1-linked (linear) diubiquitin even after overnight incubation. Finally, both CidB and CidB^{wMel} cleaved Ub-AMC, and to a much lesser extent, the UBL Nedd8-AMC (FIG. 24). The CidB-C1025A catalytic mutant was inactive against Ub-AMC (data not shown). Despite the ability to cleave multiple substrates in vitro, CidB appears to have a restricted substrate range in cells, as bulk ubiquitin conjugates in yeast were not detectably altered by CidB expression (FIG. 23c).

Because CidA binds CidB and suppresses CidB toxicity in yeast, we tested whether CidA inhibited CidB DUB activity. A 100-fold molar excess of CidA failed to inhibit CidB modification by Ub-VMe or cleavage of Ub chains (FIG. 17a, b, last lane of each panel); Ub-AMC hydrolysis also was not blocked (data not shown). CidA must block toxicity in yeast by some other means, such as control of its localization. This would have the advantage that the related CidA and CinA antidotes could be deployed against toxins with diverse enzymatic functions, such as those with DUB and nuclease domains.

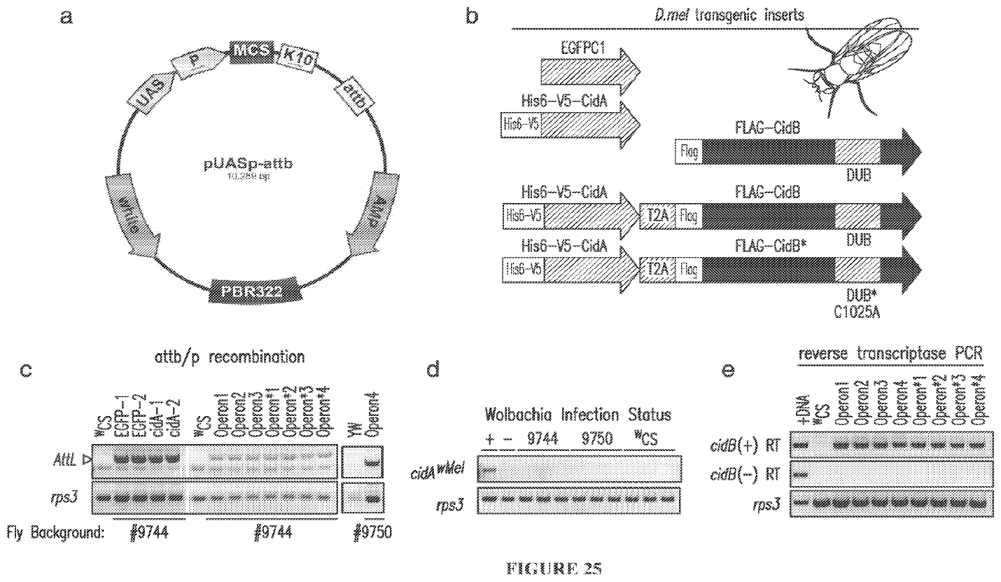

To test the ability of the cidA-cidB operon to induce CI in an insect in the absence of Wolbachia infection, we cloned expression constructs into the germline-optimized pUASp-attB vector^{32,33} for transgenic insertion into D. melanogaster by the site-directed @C31 integrase³⁴ (FIG. 25). The multiple independent transgenic flies each had a fusion of the cidA-cidB ORFs linked by a T2A viral peptide sequence that causes ribosomal skipping such that CidA and CidB are produced as separate proteins³⁵ (FIG. 25b). After transgenesis we verified attB/P recombination by PCR, confirmed that our fly lines were not infected by native Wolbachia strains, and verified transgene expression by reverse-transcription PCR (FIG. 25c-e). Males expressing the transgenic operon displayed a fully penetrant sterility in matings with wild-type females (four biological replicates with two independent attP insertion sites; FIG. 18a; FIG. 25). Females transgenic for the cidA-cidB operon were fertile, indicating that the operon caused male-specific sterility. Importantly, mutational inactivation of the CidB DUB (CidB-C1025A) in transgenic inserts failed to cause male sterility, linking CidB enzyme activity to sterility (FIG. 18a, “operon*”). Attempts to rescue the CI-like phenotype with transgenic females expressing either CidA alone or the full operon were not successful.

To verify that cidA-cidB specifically induced CI rather than an alternative form of sterility, we determined whether embryos from crosses with cidA-cidB transgenic males recapitulated established CI cytological and embryonic defects (FIG. 18b). These defects include impaired male pronuclear chromatin condensation at metaphase and delayed chromosome separation and bridging at anaphase. All were observed in the transgenic crosses. Of the embryos observed during the first post-fertilization mitosis, 88% showed these CI-like defects compared to only 3% in WT crosses (FIG. 25). Of embryos that were left to develop for 24 hours, 60% arrested “early,” prior to blastoderm formation. Of the 20% of embryos that developed to segmentation, 69% showed segmentation deformities³⁶ (FIG. 25c, d). These specific developmental defects recapitulate those of CI embryos.^{11,15-18,36,37} Thus, the defects produced by cidA-cidB expression in males closely mimic the established developmental abnormalities in CI-inducing crosses from Wolbachia-infected males.

Research on CI was pioneered 63 years ago in intraspecific crosses of the mosquito C. pipiens,^{3.38} and intracellular Wolbachia infections were described over 90 years ago.¹ The Wolbachia-CI link was made in 1971,4 but the molecular mechanism has remained obscure. Our data provide strong evidence that the Wolbachia cidA-cidB operon is responsible for CI. The most parsimonious interpretation of our yeast and transgenic fly data is an adaptation of the modification-rescue framework first proposed by Hurst¹³ and Werren¹² in which CidB would be the modifier or toxin and CidA would function as the rescue factor or antidote. Wolbachia bacteria have a type IV secretion system that could translocate the CidA and CidB proteins into the host cytoplasm.³⁹ In analogy to many toxin-antidote systems in free-living bacteria, we propose that within the fertilized egg of an incompatible cross, CidA is rapidly inactivated or degraded. Unless CidA is supplied by a maternal Wolbachia infection in the egg's cytoplasm, the paternally supplied CidB enzyme toxin will become active. However, CidA alone, might not be sufficient for rescue in the egg; additional Wolbachia or host factors might be required, possibly for co-localization of the toxin and antidote. The exact targets of the CidB DUB enzyme (and putative CinB nuclease) and the detailed molecular pathway of cidA-cidB-induced CI also remain to be determined.

Wolbachia Genomics Supports a Role for the cidA-cidB Operon in CI

The lock-and-key model, originally proposed as the toxin-antidote model by Hurst 1991,¹³ has gained traction as the model that best describes the phenomenology of CI in insects.¹⁰ Our toxin-antidote operon fits all tenets of the lock-and-key model: i) lock and key functions are genetically distinct (FIG. 15c-e); ii) independent sets of locks and keys exist; 19 iii) pairs of locks and keys interact in a specific or preferential manner (FIGS. 15f, 16b); and iv) locks and keys are co-evolving/diverging from a common ancestor (FIG. 19).19

Although we have not proven antidote function for the CidB orthologs in an insect host, genomic evidence supports our molecular specification of the lock-and-key hypothesis. Different strains of Wolbachia show different reproductive phenotypes. Wolbachia that infect Drosophila simulans show five different CI phenotypes. Specifically, three strains exhibit mutual bi-directional incompatibilities (different locks/toxins)—wRi, wHa, and wNo;²⁶ each strain has a unique toxin variant: one with an unknown enzymatic function (WRI_RS03365), one with a C48/Ulp1-like cysteine protease domain (WHA_RS01430), and one with the DUF1703 putative nuclease domain (wNo_01980), respectively. Different enzymatic toxin domains can rationalize these incompatibilities. A fourth strain, wAu, which is unable to induce or rescue CI lacks the operon altogether.⁵⁰ Finally, all sequenced genomes from so-called A and B strains that induce CI have orthologs to the putative wPip CI operon, and all strains of Wolbachia not observed to induce CI (wAu, wOo, and wBm) lack an orthologous operon.¹⁹ Therefore, all the assembled genomes of Wolbachia show a correlation between their CI phenotypes and cid/cin operon structures.

A full-length CidB structure, rather than simply presence of the DUB domain, appears to be necessary for CI. BLAST analysis of the Ulp1-like CidB domain shows that small truncated orthologs of the enzymatic Ulp1-like domain are present in non-inducing CI strains as well as non-CI inducing Rickettsial relatives. We make a distinction between these small truncated versions and full-length genes. The small versions are exemplified by the paralogous wPa_1291 of wPip, which encodes just the Ulp1-like domain and lacks possibly important N-terminal residues, an operon structure, or an associated antidote. Notably, when we analyzed wPa_1291 (which encodes residues equivalent to 894-1177 of CidB), we found that it would not induce toxicity in yeast (data not shown). This suggests that N-terminal residues and possibly even the hypothetical antidote are important for toxin localization and CI induction. In Beckmann and Fallon (2013), a toxin-antidote hypothesis was postulated in which CidA acted as toxin and CidB as antidote. This was because we had detected CidA in mature mosquito sperm purified from spermathecae.¹⁹ Our analyses in yeast and Drosophila (FIGS. 16 and 18) now suggest the opposite, namely, that CidB acts as toxin and CidA as antidote. Although CidB had not been detected within sperm, this does not repudiate our new formulation. Because CidA binds to CidB, it is possible that the CidA antidote might even play a role in localizing CidB within the mature sperm or in the zygote. We were unable to generate a transgenic line expressing just the CidB enzyme. In contrast, all other constructs could be readily inserted into the fly genome. We suspect that CidB, by itself, was killing the injected flies. The CidA protein might mitigate unwanted side effects of CidB expression. Further investigation of molecular interactions and localization of the putative toxin and antidote proteins will be needed.

Bacterial DUBs are Secretion System Effectors that Modulate Host Ubiquitin Systems

Prokaryotic ubiquitin-like protein (UBL) proteases (ULPs) and DUBs are frequently encoded by pathogenic gram-negative and obligate intracellular bacteria.²¹ This is intriguing because prokaryotes do not have their own full ubiquitin-proteasome system.⁵¹ All identified bacterial DUBs specifically tested for secretion have been shown to be secreted as effector proteins. Type III secretion system (T3SS) substrates include ChlaOTU (Chlamydia), a DUB which is thought to interact with intrinsic cellular immunity/autophagy systems regulated by ubiquitin;⁵² XopD (Xanthomonas) a SUMO protease which affects modification of important plant transcription factors by the UBL SUMO;⁵³⁻⁵⁵ and SseL (Salmonella), a DUB that was shown to be a virulence factor important for regulation of cytotoxicity in macrophages.⁵⁶ A Type IV secretion system (T4SS) substrate is SdeA (Legionella), which is essential for virulence in protozoan hosts.⁵⁷ No reports describe an intrabacterial function for any prokaryotic ULP or DUB. Because the CidA protein was detected in spermathecal tissues not known to harbor endogenous Wolbachia infections, secretion of the protein is suggested.¹⁹ Interestingly, the cidA-cidB operon was shown to be incorporated into WO prophage genomes,⁵⁸ making the translated proteins' escape from cells by phage-induced bacterial lysis, or incorporation into transmissible viral particles, another possibility. Overall, these data strongly suggest secretion of the CidA and CidB proteins, although this remains to be proven.

The CidB enzyme showed no activity toward mammalian SUMO1-AMC or SUMO2-AMC substrates or toward yeast SUMO (Smt3) fusions. Because XopD from Xanthomonas specifically targeted plant SUMO isoforms and would not cleave SUMO from other species,⁵⁹ we thought it possible that the CidB enzyme might specifically cleave Culex mosquito SUMO and not other isoforms. We cloned the mosquito SUMO as a fusion substrate with ubiquitin and tested this protein for cleavage by CidB; it did not cleave and was also inactive toward ISG15-AMC. CidB showed weak activity toward Nedd8-AMC; its k_{cat}/K_{M} for Nedd8-AMC was determined to be 0.69 μM⁻¹ min⁻¹ (FIG. 24). By comparison, the k_{cat}/K_{M} for Ub-AMC hydrolysis was 7.59 μM⁻¹ min⁻¹. CidB^{wMel} had a similar preference for ubiquitin over Nedd8 (FIG. 24). These data imply that CidB specifically targets ubiquitin linkages rather than UBL conjugates; weak cross reactivity with Nedd8 is more likely an off-target effect due to the close sequence similarity of Nedd8 and ubiquitin.²¹ All these data support the hypothesis that the major biological effects of CidB are mediated by its activity against ubiquitin conjugates. Identification of its critical in vivo substrates will be needed to test this model.

We investigated the ability of CidB to cleave all seven possible ubiquitin-C-terminus-lysine linkages in ubiquitin dimers as well as the linear Met1-ubiquitin linkage because different ubiquitin chains of different linkages are associated with different cellular pathways.⁶⁰ CidB displayed activity towards all of the lysine-linked diubiquitins but was unable to cleave linear diubiquitin in 1 h or overnight at 37° C. Other DUBs, mainly from the USP family, such as USP7 and USP28, are similarly active against multiple chain linkages but not linear diubiquitin.⁴¹ Of all the possible linkages explored in our diubiquitin panel digest (FIG. 23a), CidB appeared to have the highest activity toward Lys48 and Lys63. The Lys48 polyubiquitin linkage often signals for substrate degradation by the proteasome, whereas Lys63 linkages are typically involved in certain DNA repair pathways and endocytosis.²¹ The preference of CidB for K63 ubiquitin dimers over K48 dimers is relatively modest (˜4-fold), so we cannot conclude which chain types might be most relevant to CI induction, although K63 chain-modified (or monoubiquitylated) substrates appear most likely.

It has been speculated that CI targets a core conserved biochemical machinery involved in mitosis because delays in chromosome condensation and bridging are, without exception, observed in insects ranging from mosquitoes (Culex and Aedes), fruitflies (Drosophila), and wasps (Nasonia).^{11,15} Furthermore, artificial transfection of heterologous Wolbachia strains into diverse hosts still results in induction of CI (wAlbB into Anopheles stephensi:²⁷ wRi into Drosophila melanogaster;²⁸ wMel into Aedes aegypti).²⁹

Our data with heterologous expression of the Wolbachia cid and cin genes in yeast fully support this idea of broad host range. Similarly, we could induce robust transgenic CI in Drosophila flies with an operon from a Wolbachia strain that normally infects Culex mosquitoes. This CI-like effect over a broad host range also means that the transgenic operon might be utilized in many different insect pests or disease vectors to limit their populations.

Finally, CidB is not the only means of inducing CI. There are redundant paralogous operons, such as in wPip. In the case of wNo, which lacks a functional cidA-cidB operon, CI may be induced by virtue of the orthologous DUF1703 nuclease-type operon. The DUF1703 domain has previously been implicated in insect sterility.⁶³ Likewise, in wPip both paralogous operons might induce CI simultaneously, creating multi-directional incompatibility dynamics (peptides were detected from both operon systems in an ovarian proteome).⁶²

In accord with the lock-and-key model, the two paralogous operons appear to share a common ancestor. Not only does conservation of sequence suggest this, but secondary structure predictions from Psipred⁶⁴ show that the CidB proteins share an underlying CinB-related secondary structure immediately preceding the DUB domain (FIG. 19; αβββα⊕).²⁵ In contrast to the CinB-type operons, the CidB operons do not maintain conservation of the D-E-K catalytic triad predicted to coordinate a metal ion for nuclease activity. However, the underlying structural skeleton suggests that a common ancestor was a nuclease form which then diverged by addition of the DUB domain at its C-terminus and mutational drift of the nuclease active site.

Interestingly, a divergent version of the apparent CI toxin from Rickettsia gravesii has both a DUF1703 nuclease and a DUB domain (WP_024547315.1). This ortholog may be an evolutionary “missing link” between the paralogous forms diverging in Wolbachia. Furthermore, another known CI-inducing bacterium, the phylogenetically distant Cardinium hertigii, was shown to possess a USP-type DUB in its genome, making it a possibility that Cardinium uses this effector to induce CI by a similar pathway.⁶⁵

### Methods

### DNA Manipulation

DNA was purified from Wolbachia-infected insects according to Beckmann and Fallon 2012.42 Genes from cid and cin operons were cloned from DNA of wPip-infected C. pipiens Buckeye mosquitoes¹⁹ and from YW wMel-infected D. melanogaster flies. PCR products were amplified using PhusionHF DNA polymerase (New England Biolabs), gel-purified, and ligated into various plasmid vectors, including the pBAD (ThermoFisher; arabinose induction), pET (ThermoFisher; IPTG induction), pCold-GST (gift from Chittaranjan Das; IPTG induction) and pGEX (GE Healthcare; IPTG induction) E. coli expression vectors. All plasmid inserts were fully sequenced at the Yale Keck Foundation DNA sequencing facility. Point mutations were introduced by QuikChange mutagenesis (Stratagene). Further modifications such as truncations or tag additions were carried out using SLIM.⁴³

### Protein Purification for Pulldown Analysis of His6-Tagged Proteins

The procedure followed was a slight modification of the Dynabeads manufacturer's protocol (Novex). Recombinant proteins were expressed in E. coli strains BL21-AI (ThermoFisher) or Rosetta DE3 (Novagen). Large (2 L) or small (100 ml) cultures were grown in Luria Broth (LB) at 37° ° C. with vigorous shaking to 0.5 OD at λ 600 nm and induced by either 0.02% arabinose (pBAD) or 1 mM IPTG (PET). Protein induction in most cases was allowed to proceed overnight at 18° C. Cell pellets were resuspended in binding wash buffer (50 mM sodium phosphate [pH 8.0]; 300 mM NaCl; 0.01% Tween-20; 5 mM-mercaptoethanol; 10 mM imidazole) and lysed by either sonication or French press. Cell lysates were incubated for 10-60 min at 4° C. with HisPur cobalt resin or Ni-NTA agarose resin (both Qiagen).

For His6-tagged protein pulldown assays, bead-bound tagged proteins were incubated with bacterial extracts containing bait protein for 1 h at 4° ° C. The resin was washed, and bound proteins were eluted at 4° C. with 1 bead volume of elution buffer containing 300 mM imidazole. For large-scale purifications of His6-tagged proteins, eluates isolated by the same method were concentrated to ˜0.3 ml in a 10 Kda cutoff concentrator (Amicon). Protein concentrations were determined either by densitometry on a Syngene G:box with GeneTools software using BSA as a standard or by Bradford assay (Bio-Rad). We note that in FIG. 15f, protein loading of the CidB^{wMel} toxin was lower than the others because it expressed at very low levels.

Purification of Proteins for Kinetic Assays

To obtain purified enzymes for kinetic analysis of DUB activity, CidB(762-1143) and CidB^{wMel} (797-1128) were overproduced as glutathione-S-transferase (GST) fusions in E. coli with minor modifications to the protocol described previously.⁴⁴ Briefly, large-scale cultures were grown to late exponential phase in LB and were induced with 0.3 mM IPTG. Following induction at 37° C. for 4 h, cells were harvested and lysed with a French press. Proteins were purified by GST-affinity chromatography using glutathione agarose (Thermo Scientific). After removal of the GST tag with PreScission protease (GE Biosciences), the protein was further purified by size-exclusion chromatography using a HiLoad Superdex S75 PG column (GE Biosciences) in a buffer consisting of 50 mM Tris-HCl (pH 7.6), 150 mM NaCl, and 1 mM DTT. All protein samples were concentrated, aliquoted, flash frozen, and stored at −80° ° C. until use. Prior to use, concentrations were carefully determined both spectrophotometrically at 280 nm and by BCA Assay (Thermo Scientific).

Lys63-linked and Lys48-linked ubiquitin dimers were synthesized enzymatically using Lys63Arg, Lys48Arg, and Asp77 (mouse) ubiquitin mutants according to a previously described method.^{44,45} Enzymes required for formation of Lys63 diubiquitin were human E1 (pGEX6P1 vector), Uevla (pGEX6P1), Ubc13 (pGEX6P1), Lys63Arg ubiquitin (pET26b), and Asp77 ubiquitin (pET26b). These were purified separately and mixed in a reaction buffer containing 80 mM Tris-HCl (pH 7.6), 20 mM ATP, 20 mM MgCl₂, and 1 mM DTT. Synthesis of Lys48 diubiquitin used a reaction consisting of human E1, CDC34 (pET16b), Lys48Arg ubiquitin (pET26b) and Asp77 ubiquitin. All reactions proceeded overnight at room temperature and were quenched by addition of a 10-fold excess of Buffer A [50 mM NaOAC (pH 4.5)]. Unreacted ubiquitin and enzymes utilized for the reaction were separated from newly formed diubiquitin using MonoS cation-exchange chromatography (GE Biosciences). Lys63- and Lys48-linked ubiquitin dimers were eluted using a linear gradient of Buffer A mixed with Buffer B [50 mM NaOAc (pH 4.5), 1 M NaCl], and then buffer exchanged to 50 mM Tris-HCl (pH 7.6), 150 mM NaCl, 1 mM DTT. All diubiquitin samples were concentrated, aliquoted, flash frozen, and stored at −80° C. until use.

### SDS-Polyacrylamide Gel Electrophoresis and Western Immunoblotting

Standard SDS-PAGE gel analysis was carried out in a range of gel concentrations. Proteins were either stained with GelCode Blue (ThermoFisher) or transferred to PVDF Immobilon-P transfer membranes (0.45 μM pore size) (SigmaAldrich) for immunoblot analysis.⁴⁶ Antibodies utilized for immunoblotting were: mouse anti-tetraHis (Qiagen, 1:4,000); mouse anti-FLAG M2 (Sigma, 1:10,000); rabbit anti-ubiquitin (Dako, 1:1000); mouse 16B12 anti-HA (BAbCO Covance, 1:1000); and mouse anti-PGK (yeast phosphoglycerate kinase) (Molecular Probes, 1:20,000). Secondary antibodies used were: sheep anti-mouse NA931V (GE Healthcare, 1:10,000) and donkey anti-rabbit NA934V (GE Healthcare, 1:5,000). Membranes used for anti-His blotting required blocking of nonspecific binding with 3% BSA and extensive washing. Other immunoblot analyses used 5% milk for blocking.

### Diubiquitin Cleavage Assays

Chain cleavage assays were carried out using CidB(762-1143) following a previously published protocol.⁴⁴ Briefly, 250 nM CidB was incubated in a reaction buffer of 50 mM Tris (pH 7.6), 20 mM KCl, 5 mM MgCl₂, and 1 mM DTT with Lys63-linked diubiquitin concentrations ranging from 20-120 μM. In assays using Lys48-linked diubiquitin, 400 nM CidB was used. All reactions were carried out at room temperature for 10 min (Lys63 reactions) or 15 min (Lys48 reactions) and were quenched by the addition of 5×SDS-PAGE sample buffer. Ubiquitin standards ranging from 6-40 μM were used to generate a standard curve, enabling quantification of ubiquitin produced from each diubiquitin cleavage reaction using ImageJ software.⁴⁷ To account for the release of two ubiquitin moieties (P and P′) from a single reaction, the initial rates of each reaction were divided by 2. All kinetic data were analyzed with Kaleidagraph Version 4.1.3b1 and could be fit to the Michaelis-Menten equation: Vᵢ=(Vₘₐₓ [S])/(K_{M}+[S]) where [S] is the concentration of substrate. We also tested reactivity of full length CidB with all seven potential ubiquitin lysine linkages by incubating 1 μM enzyme with 1 μM diubiquitin for 3 h or overnight at 37° C. using the Ub2 Explorer Panel (LifeSensors). Lastly, we incubated 50 nM CidB with 500 nM mixtures of Lys63-linked or Lys48-linked polyubiquitin chains (ranging in size from 2-7 ubiquitins; Boston Biochem) for times of 20 min to 4 h at 37° C. Error bars are standard deviations.

### Ubiquitin-AMC and UBL-AMC Hydrolysis Assays

Ubiquitin (Ub) and ubiquitin-like protein (UBL) with C-terminal 7-amido-4-methylcoumarin adducts (Ub-AMC and UBL-AMC) were used for hydrolysis assays as described previously.⁴⁰ Briefly, a CidB fragment encompassing the DUB domain (residues 762-1143) was diluted to a final concentration of 5 nM in reaction buffer (50 mM Tris, pH 7.6, 0.5 mM EDTA, 0.1% bovine serum albumin, 5 mM DTT). Prior to addition of the Ub or UBL-linked AMC substrate (Ub-AMC, NEDD8-AMC, SUMO1/2-AMC, and ISG15-AMC; Boston Biochem), the enzyme was pre-incubated at 30ºC for 5 min, and all reactions proceeded at 30° C. Apart from the ISG15-AMC substrate (excitation/emission 380 nm/460 nm), hydrolysis of the Ub/UBL-AMC substrates as a function of time was monitored via excitation/emission at 345 nm/445 nm using a SynergyMix plate reader (BioTek, Winooski, VT). A standard curve comprising AMC (Sigma Aldrich) concentrations ranging from 0-50 nM was prepared in reaction buffer to allow quantification of the amount of hydrolyzed substrate. Despite testing human ISG15-AMC and SUMO1/2-AMC with several concentrations of CidB (up to 400 nM), we failed to detect any AMC release. Substrate concentrations ranging from 50 nM to 2 μM were mixed with 5 nM and 25 nM CidB in Ub-AMC and Nedd8-AMC assays, respectively. Initial velocities were extrapolated from the linear portion of the curve and plotted as a function of substrate concentration. As the catalytic activity exhibited a linear response to substrate over the concentration range tested, data could not be fit to the Michaelis-Menten equation. Data were instead fit to the equation v/[E]=k_{cat}/K_{M}[S], where [E] and [S] are the concentrations of enzyme and substrate, respectively. All enzymatic assays were carried out in triplicate and analyzed using Kaleidagraph Version 4.1.3b1. Error bars are standard deviations.

### Generation of a Covalent CidB-UbVME Adduct

To test for formation of a covalent complex between CidB and the suicide DUB inhibitor UbVME, 5 μM CidB was mixed with 1 μM HA-UbVME (a gift from Michael Sheedlo and Chittaranjan Das, Purdue University). After adjusting the pH to 8, reactions were carried out for 4 h at 37° C. and quenched by mixing with 5×SDS sample buffer, and the products were run on a gradient SDS-PAGE gel. Following electrotransfer to a PVDF filter, the filter was incubated, as outlined above, with anti-HA antibodies, followed by secondary antibody.

### Yeast Methods

Analysis of yeast growth that is displayed in figures utilized the BY4741 strain background. Rescue experiments were replicated in the W303a background. DNA fragments used for expression in yeast were subcloned from E. coli vectors by restriction digest or PCR amplification and ligated into yeast vectors. The 2-micron plasmids pYES2 (URA3) and p425GAL (LEU2) both had the GAL1 promoter and CYC1 terminator and were utilized for galactose-induced expression of Wolbachia genes in yeast.⁴⁸ Expression from the low-copy CEN vector pRS416 was also utilized. For serial dilutions of yeast cells, cultures were grown overnight in non-inducing minimal synthetic media lacking either uracil, leucine, or both depending upon the plasmid(s) used for expression. Cells were pelleted by centrifugation, washed with sterile water, and spotted in 5-fold serial dilution from an initial 0.05 OD600 concentration on solid minimal SD media containing either 2% galactose or glucose and lacking either uracil, leucine, or both. Plates were placed at 30, 32, 34, and 37° C. for 3 d.

### Drosophila Genetic Analysis

An initial cidA-T2A-cidB operon construct was synthesized and codon optimized for Drosophila by Genscript and cloned into the pUC57 vector (FIG. 25b). Genes were then subcloned from the mother construct into the pUASp-attB vector^{32,33} by PCR and restriction digest. The full-length operon construct pUASp-attB-cidA-T2A-cidB was unstable in TOP1OF′ bacterial cells and prone to degradation. The plasmid was stabilized in CopyCutter EPI400 cells (Epicentre). All constructs for transgenesis in the pUASp-attB vector were fully sequenced and verified to lack spurious mutations.

DNA constructs were sent to BestGene for microinjection of D. melanogaster embryos. Fly backgrounds #9744 and #9750 (containing attP insertion sites on the 3^{rd} chromosome) were chosen for site-directed attP/B integration by the ΦC31 integrase. Red-eyed flies were selected and screened by BestGene. Upon receipt of transgenic lines, we independently verified attP/B integration by PCR using primers 509 (5′-GGGCGTGCCCTTGAGTTCTCTC-3′; SEQ ID NO:21) and 510 (5′-CGAGGATCGCATACCGCACTG-3′; SEQ ID NO:22) (#9744; 0.5 kb product) or 509 (5′-GGGCGTGCCCTTGAGTTCTCTC-3′; SEQ ID NO:23) and 511 (5′-AACGCTTTGCTTTCTCGCTG-3′; SEQ ID NO:24) (#9750; 0.7 kb product), which amplified a product only if site-specific recombination had occurred. We also verified that our #9744, #9750, and ^{w}CS strains were uninfected with native Wolbachia isolates that might interfere with crossing data. This was done using PCR to amplify the cidA^{wMel} gene. As a positive DNA control, we amplified a ˜200 bp product of D. melanogaster rps3. The basal P-element promoter in pUASp-attB induced sufficient expression to induce phenotypes without a Gal4 driver. This was confirmed by reverse transcription-PCR (RT-PCR) analysis carried out by purifying RNA with TRIzol reagent (Ambion) according to the manufacturer's specifications from pools of 20 male flies. RNA was further purified with by RNeasy (Qiagen) and treated with DNase I. Complementary DNA was synthesized using the isCript cDNA Synthesis Kit (BioRad), and the cDNA was used as template for PCR reactions with primers that amplified either CidB or rps3.

Flies were maintained at room temperature on a standard diet. For CI analysis, two males (<3 d old) were mated to 10 virgin females in an individual tube. 1 tube of 12 flies was one N. Adult flies were removed after 10 days of egg laying, and fecundity was assessed by counting eclosions of adult progeny. In the case of the crosses that led to sterility, flies were allowed to lay eggs until they died in the tube; they never produced offspring. To assess the cytology of early embryos resulting from an incompatible cross with cidA-cidB transgenic males, ˜300 virgin female ^{w}CS flies were placed in a collection container with ˜100 transgenic cidA-cidB males and put on apple juice plates with yeast paste for 2 d. Embryos were then collected by a brush and sieve every 15 min, dechorionated in 50% bleach, and fixed immediately in a solution of 5 ml heptane, 2 ml 2.5×PBS, 500 μl 0.5 M EDTA, and 1 ml of 37% fresh formaldehyde. The fixing solution (10 ml) was kept in a clear glass scintillation vial to allow visualization of liquid phase layers and eggs. Vitelline membranes were removed by replacing the heptane top layer with 2 volumes of methanol and vigorous shaking. Sunken de-vitellinated embryos were collected with a Pasteur pipette, washed three times with methanol, and stored overnight at 4° C. before they were rehydrated with PBTA⁴⁹ and stained with Hoechst 33342 dye (ThermoFisher Scientific) at 1:1000 in PBTA. Stained embryos were washed and mounted on glass slides and sealed under a cover slip with nail polish. Microscopic analysis of the embryos was performed on a Zeiss Axioskop microscope using a 100X/1.4 NA objective lens.

Variations in the cytological quantifications are shown as the standard deviation of the mean of triplicate samples of 200 embryos (FIG. 25a). Polar bodies were used as a landmark. Images where polar bodies were not observed were excluded from the data (FIG. 25b). Images were captured by AxioVision Re.4.8 software and adjusted for contrast and assembled in Photoshop (Adobe). The images confirmed that the cidA-cidB transgenic males, while sterile, mated and successfully fertilized eggs. In cases where nuclei were not well visualized in a single plane of focus, a Z-stack maximum projection was created in ImageJ.

Crosses aimed at testing rescue of cidA-cidB-induced lethality were performed by first creating various heterozygous [GAL4; UAS-CidA] flies. These were generated by crossing [yw; UAS-CidA] homozygous virgin females with male driver strains that are expected to express Gal4 during oogenesis: #4442: nanos-Gal4, #32551: ubiquitin-Gal4, #44241: oskar-Gal4, #7062: MATα-Ga14 (all transgenes on the 2^{nd} chromosome), or #31777: MTD-Gal4, which has many Gal4 inserts on all three chromosomes including nanos-Gal4, nanos-Gal4: VP16, and otu-Gal4. These double heterozygotes were then mated with cidA-cidB males to test fecundity. Fly stocks were obtained from the Bloomington Stock Center or were gifts.

### REFERENCES CITED IN EXAMPLE 2

1 Hertig, M. & Wolbach, S. B. Studies on Rickettsia-like micro-organisms in insects. J Med Res 44, 329-U322 (1924).
2 Werren, J. H., Baldo, L. & Clark, M. E. Wolbachia: master manipulators of invertebrate biology. Nat Rev Microbiol 6, 741-751 (2008).
3 Laven, H. Chapter 7: Speciation and Evolution in Culex pipiens. 251 (Elselvier, 1967).
4 Yen, J. H. & Barr, A. R. New hypothesis of the cause of cytoplasmic incompatibility in Culex pipiens L. Nature 232, 657-658 (1971).
5 Bourtzis, K., Braig, H. R., and Karr, T. L. Chapter 14 Cytoplasmic Incompatibility. Vol. 1 (CRC Press, 2003).
6 Turelli, M. & Hoffmann, A. A. Rapid spread of an inherited incompatibility factor in California Drosophila. Nature 353, 440-442 (1991).
7 Bordenstein, S. R., O'Hara, F. P. & Werren, J. H. Wolbachia-induced incompatibility precedes other hybrid incompatibilities in Nasonia. Nature 409, 707-710 (2001).
8 Clark, M. E., Veneti, Z., Bourtzis, K. & Karr, T. L. Wolbachia distribution and cytoplasmic incompatibility during sperm development: the cyst as the basic cellular unit of CI expression. Mech Dev 120, 185-198 (2003).
9 Presgraves, D. C. A genetic test of the mechanism of Wolbachia-induced cytoplasmic incompatibility in Drosophila. Genetics 154, 771-776 (2000).
10 Poinsot, D., Charlat, S. & Mercot, H. On the mechanism of Wolbachia-induced cytoplasmic incompatibility: confronting the models with the facts. BioEssays: news and reviews in molecular, cellular and developmental biology 25, 259-265 (2003).
11 Serbus, L. R., Casper-Lindley, C., Landmann, F. & Sullivan, W. The Genetics and Cell Biology of Wolbachia-Host Interactions. Annual review of genetics 42, 683-707 (2008).
12 Werren, J. H. Biology of Wolbachia. Annu Rev Entomol 42, 587-609 (1997). 12
13 Hurst, L. D. The Evolution of Cytoplasmic Incompatibility or When Spite Can Be Successful. J Theor Biol 148, 269-277 (1991).
14 Loppin, B., Dubruille, R. & Horard, B. The intimate genetics of Drosophila fertilization. Open biology 5 (2015).
15 Tram, U., Ferree, P. M. & Sullivan, W. Identification of Wolbachia—host interacting factors through cytological analysis. Microbes and infection/Institut Pasteur 5, 999-1011 (2003).
16 Callaini, G., Dallai, R. & Riparbelli, M. G. Wolbachia-induced delay of paternal chromatin condensation does not prevent maternal chromosomes from entering anaphase in incompatible crosses of Drosophila simulans. Journal of cell science 110 (Pt 2), 271-280 (1997).
17 Reed, K. M. & Werren, J. H. Induction of paternal genome loss by the paternal-sex-ratio chromosome and cytoplasmic incompatibility bacteria (Wolbachia): a comparative study of early embryonic events. Molecular reproduction and development 40, 408-418 (1995).
18 Ryan, S. L. & Saul, G. B., 2^{nd}. Post-fertilization effect of incompatibility factors in Mormoniella. Molecular & general genetics: MGG 103, 29-36 (1968).
19 Beckmann, J. F. & Fallon, A. M. Detection of the Wolbachia protein WPIP0282 in mosquito spermathecae: implications for cytoplasmic incompatibility. Insect biochemistry and molecular biology 43, 867-878 (2013).
20 Hochstrasser, M. Ubiquitin-dependent protein degradation. Annual review of genetics 30, 405-439 (1996).
21 Ronau, J. A., Beckmann, J. F. & Hochstrasser, M. Substrate specificity of the ubiquitin and Ubl proteases. Cell research (2016).
22 Zielenkiewicz, U. & Ceglowski, P. Mechanisms of plasmid stable maintenance with special focus on plasmid addiction systems. Acta biochimica Polonica 48, 1003-1023 (2001).
23 Yamaguchi, Y., Park, J. H. & Inouye, M. Toxin-antitoxin systems in bacteria and archaea. Annual review of genetics 45, 61-79 (2011).
24 O'Neill, S. L. & Karr, T. L. Bidirectional incompatibility between conspecific populations of Drosophila simulans. Nature 348, 178-180 (1990).
25 Knizewski, L., Kinch, L. N., Grishin, N. V., Rychlewski, L. & Ginalski, K. Realm of PD-(D/E)XK nuclease superfamily revisited: detection of novel families with modified transitive meta profile searches. BMC structural biology 7, 40 (2007).
26 Mercot, H. & Charlat, S. Wolbachia infections in Drosophila melanogaster and D. simulans: polymorphism and levels of cytoplasmic incompatibility. Genetica 120, 51-59 (2004).
27 Bian, G. et al. Wolbachia invades Anopheles stephensi populations and induces refractoriness to Plasmodium infection. Science 340, 748-751 (2013).
28 Boyle, L., O'Neill, S. L., Robertson, H. M. & Karr, T. L. Interspecific and intraspecific horizontal transfer of Wolbachia in Drosophila. Science 260, 1796-1799 (1993).
29 Ye, Y. H. et al. Wolbachia Reduces the Transmission Potential of Dengue-Infected Aedes aegypti. PLOS neglected tropical diseases 9, e0003894 (2015).
30 Xi, Z., Khoo, C. C. & Dobson, S. L. Wolbachia establishment and invasion in an Aedes aegypti laboratory population. Science 310, 326-328 (2005).
31 Li, S. J. & Hochstrasser, M. A new protease required for cell-cycle progression in yeast. Nature 398, 246-251 (1999).
32 Rorth, P. Gal4 in the Drosophila female germline. Mech Dev 78, 113-118 (1998).
33 Takeo, S. et al. Shaggy/glycogen synthase kinase 3beta and phosphorylation of Sarah/regulator of calcineurin are essential for completion of Drosophila female meiosis. Proceedings of the National Academy of Sciences of the United States of America 109, 6382-6389 (2012).
34 Groth, A. C., Fish, M., Nusse, R. & Calos, M. P. Construction of transgenic Drosophila by using the site-specific integrase from phage phiC31. Genetics 166, 1775-1782 (2004).
35 Diao, F. & White, B. H. A novel approach for directing transgene expression in Drosophila: T2A-Gal4 in-frame fusion. Genetics 190, 1139-1144 (2012).
36 Callaini, G., Riparbelli, M. G., Giordano, R. & Dallai, R. Mitotic defects associated with cytoplasmic incompatibility in Drosophila simulans. J Invertebr Pathol 67, 55-64 (1996).
37 Landmann, F., Orsi, G. A., Loppin, B. & Sullivan, W. Wolbachia-mediated cytoplasmic incompatibility is associated with impaired histone deposition in the male pronucleus. PLOS pathogens 5, e1000343 (2009).
38 Laven, H. [Reciprocally differentiable crossing of mosquitoes (Culicidae) and its significance for plasmatic heredity]. Z Indukt Abstamm Vererbungsl 85, 118-136 (1953).
39 Rances, E., Voronin, D., Tran-Van, V. & Mavingui, P. Genetic and functional characterization of the type IV secretion system in Wolbachia. Journal of bacteriology 190, 5020-5030 (2008).
40 Morrow, M. E. et al. Stabilization of an unusual salt bridge in ubiquitin by the extra C-terminal domain of the proteasome-associated deubiquitinase UCH37 as a mechanism of its exo specificity. Biochemistry 52, 3564-3578 (2013).
41 Ritorto, M. S. et al. Screening of DUB activity and specificity by MALDI-TOF mass spectrometry. Nat Commun 5, 4763 (2014).
42 Beckmann, J. F. & Fallon, A. M. Decapitation Improves Detection of Wolbachia pipientis (Rickettsiales: Anaplasmataceae) in Culex pipiens (Diptera: Culicidae) Mosquitoes by the Polymerase Chain Reaction. J Med Entomol 49, 1103-1108 (2012).
43 Chiu, J., March, P. E., Lee, R. & Tillett, D. Site-directed, Ligase-Independent Mutagenesis (SLIM): a single-tube methodology approaching 100% efficiency in 4 h. Nucleic acids research 32, e174 (2004).
44 Shrestha, R. K. et al. Insights into the mechanism of deubiquitination by JAMM deubiquitinases from cocrystal structures of the enzyme with the substrate and product. Biochemistry 53, 3199-3217 (2014).
45 Sheedlo, M. J. et al. Structural basis of substrate recognition by a bacterial deubiquitinase important for dynamics of phagosome ubiquitination. Proceedings of the National Academy of Sciences of the United States of America 112 (2015).
46 Mruk, D. D. & Cheng, C. Y. Enhanced chemiluminescence (ECL) for routine immunoblotting: An inexpensive alternative to commercially available kits. Spermatogenesis 1, 121-122 (2011).
47 Schneider, C. A., Rasband, W. S. & Eliceiri, K. W. NIH Image to ImageJ: 25 years of image analysis. Nat Methods 9, 671-675 (2012).
48 Guthrie, C. a. F., G. Guide to yeast genetics and molecular biology. Methods Enzymol 194, 1-863 (1991).
49 Sullivan, W., Ashburner, M., and Hawley, R. S. Drosophila Protocols. (Cold Spring Harbor Laboratory Press, 2000).
50 Sutton, E. R., Harris, S. R., Parkhill, J. & Sinkins, S. P. Comparative genome analysis of Wolbachia strain wAu. BMC genomics 15, 928 (2014).
51 Iyer, L. M., Burroughs, A. M. & Aravind, L. The prokaryotic antecedents of the ubiquitin-signaling system and the early evolution of ubiquitin-like beta-grasp domains. Genome biology 7, R60 (2006).
52 Furtado, A. R. et al. The chlamydial OTU domain-containing protein ChlaOTU is an early type III secretion effector targeting ubiquitin and NDP52. Cellular microbiology 15, 2064-2079 (2013).
53 Hotson, A., Chosed, R., Shu, H., Orth, K. & Mudgett, M. B. Xanthomonas type III effector XopD targets SUMO-conjugated proteins in planta. Molecular microbiology 50, 377-389 (2003).
54 Kim, J. G., Stork, W. & Mudgett, M. B. Xanthomonas type III effector XopD desumoylates tomato transcription factor SIERF4 to suppress ethylene responses and promote pathogen growth. Cell host & microbe 13, 143-154 (2013).
55 Noel, L., Thieme, F., Nennstiel, D. & Bonas, U. Two novel type III-secreted proteins of Xanthomonas campestris pv. vesicatoria are encoded within the hrp pathogenicity island. Journal of bacteriology 184, 1340-1348 (2002).
56 Rytkonen, A. et al. SseL, a Salmonella deubiquitinase required for macrophage killing and virulence. Proceedings of the National Academy of Sciences of the United States of America 104, 3502-3507 (2007).
57 Bardill, J. P., Miller, J. L. & Vogel, J. P. IcmS-dependent translocation of SdeA into macrophages by the Legionella pneumophila type IV secretion system. Molecular microbiology 56, 90-103 (2005).
58 Bordenstein, S. R. & Bordenstein, S. R. Lateral genetic transfers between eukaryotes and bacteriophages. bioRxiv, doi: 10.1101/049049 (2016).
59 Chosed, R. et al. Structural analysis of Xanthomonas XopD provides insights into substrate specificity of ubiquitin-like protein proteases. The Journal of biological chemistry 282, 6773-6782 (2007).
60 Kulathu, Y. & Komander, D. Atypical ubiquitylation—the unexplored world of polyubiquitin beyond Lys48 and Lys63 linkages. Nature reviews. Molecular cell biology 13, 508-523 (2012).
61 Tracey, W. D., Jr., Ning, X., Klingler, M., Kramer, S. G. & Gergen, J. P. Quantitative analysis of gene function in the Drosophila embryo. Genetics 154, 273-284 (2000).
62 Beckmann, J. F. Molecular Mechanism of Wolbachia Induced Cytoplasmic Incompatibility PhD thesis, University of Minnesota, (2014).
63 Lorenzen, M. D. et al. The maternal-effect, selfish genetic element Medea is associated with a composite Tc1 transposon. Proceedings of the National Academy of Sciences of the United States of America 105, 10085-10089 (2008).
64 McGuffin, L. J., Bryson, K. & Jones, D. T. The PSIPRED protein structure prediction server. Bioinformatics 16, 404-405 (2000).
65 Penz, T. et al. Comparative genomics suggests an independent origin of cytoplasmic incompatibility in Cardinium hertigii. PLOS genetics 8, e1003012 (2012).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

TABLE 6
Sequences of CI Factors

| | Locus | Seq. | | |
|---|---|---|---|---|
| Gene | tag | type | Length | (5′-3′) or (N-C) |
| cidA | WD0631 | nucle- | 1425 bp | ATGCCAATAGAAACAAAACGTCAGGCTGAAGTGCTTAAAAAGCTA |
| | | otide | | CAAGATGTGATAAAACATACAGATCGTGACATTGCGGCTGGAAGA |
| | | | | AAGTTAGCTATTAAAAGGTGGGTCGAGACCTATATAGAGTATATC |
| | | | | AAACTTTTTAAGGATGATAAGCTGGAATTCTTATATAATGTTTTTC |
| | | | | GAGATGAAGGTTGTTGGTTAGGTACAAGGTTAAATAATACTGTTTT |
| | | | | AGGTCAGAAATTGACTGAAGAGAAAATAGGAGAAATCGATAACC |
| | | | | CACTACCAAGGTATGGTATGGCATCTAGGTACTGTATAACGGGCA |
| | | | | AGATAGGTGATTTTTTCAACAAACAGTTTGTACTCTCTAGAGGTCA |
| | | | | ATTTACTTCAGAAGAGGTAGATAGTCAAGGTAATCCGATCAGTGA |
| | | | | TCAATATGTAAGAAACATTCTGCTATCATCCATGAAGAGAAATGG |
| | | | | TCCTGTGTTTGATTTCTGGATCGATAGAGAATCTGGGGAATTAAAG |
| | | | | AAGTATGATGCAGTAGAAGGTTTTGACAGTACTGTAAAACTTAAG |
| | | | | TGGAGCGAAGGGGTAGAGTATTTTTATAATCAGTTAGAGGAAAAA |
| | | | | GATAAGGAGAAGAAGCTTACAGAAGCTATTGTTGCTCTTTCTCGTC |
| | | | | CTCAATCTGTTAAGAGAGACGCTCCTATTTTAGATTTTTGTGTAAG |
| | | | | GAATATAGGCGATAAAGATACTCTTTTACAGAAATTATTGCAGAA |
| | | | | AGATAAGGGAGTATATTTCCTTCTTGCTGAATTAATAGAGTCATGT |
| | | | | TTTTTTGATACGGTTCATGATTTGGTACAGTGCTGGTGTTATAAAG |
| | | | | GCGTTTCAGCAGGAGGAGACTGTTCGGACAAGATATTCTCACAGC |
| | | | | AAGACTATGAACTTTTTCTTTATTCACTTTCAAATGTGATGTTGAA |
| | | | | AAATCCTGAGTTAAGTGTTCAAGCTAGATCCCTTATTATGGAGATT |
| | | | | TGGAAATGTGAACGCTTTGCTGAATACAGAGAGACCTCTGTTAAT |
| | | | | ACTTCTAATTATACAGTTCCTATAAAGAGTGTACTTGGGGGATTAA |
| | | | | TCATTAATTGGAAACGAGAAGATGTTTGTAAGCCCGATAGGGAAA |
| | | | | TAGAGAAAGAAGAAATATTAGATATGATTTCATTTGCCAAAGGTT |
| | | | | GCTTTCCTGAAAAGTTTGACCTTTTTAAAGAAGTCATGATAGAAAA |
| | | | | TCTTAGAATATGTGGTAGGGAAGGAAAGAGGAAAGGTGTAGATTA |
| | | | | CGGCAAGTTTGCAGAAGAGTTATTTCTTCAGTTAGAGAAAGTAAC |
| | | | | TTTACCTTCTGTAGGTGATGGTCCTTGGAATAATTTGCGGTCTCAA |
| | | | | TCTAAGGTATCTTTGCCACTTGATGGTTCTGGTGATGGCCCACAGT |
| | | | | CTGAGTTTGAAGCTCCTAGTGTGAGTGGTATTTCTGGTTCTCATAA |
| | | | | GAAAAGAAGAATCTAG (SEQ ID NO: 1) |
| cidA | WD0631 | amino | 474 aa | MPIETKRQAEVLKKLQDVIKHTDRDIAAGRKLAIKRWVETYIEYIKLF |
| | | acid | | KDDKLEFLYNVFRDEGCWLGTRLNNTVLGQKLTEEKIGEIDNPLPRY |
| | | | | GMASRYCITGKIGDFFNKQFVLSRGQFTSEEVDSQGNPISDQYVRNIL |
| | | | | LSSMKRNGPVFDFWIDRESGELKKYDAVEGFDSTVKLKWSEGVEYF |
| | | | | YNQLEEKDKEKKLTEAIVALSRPQSVKRDAPILDFCVRNIGDKDTLLQ |
| | | | | KLLQKDKGVYFLLAELIESCFFDTVHDLVQCWCYKGVSAGGDCSDKI |
| | | | | FSQQDYELFLYSLSNVMLKNPELSVQARSLIMEIWKCERFAEYRETSV |
| | | | | NTSNYTVPIKSVLGGLIINWKREDVCKPDREIEKEEILDMISFAKGCFP |
| | | | | EKFDLFKEVMIENLRICGREGKRKGVDYGKFAEELFLQLEKVTLPSVG |
| | | | | DGPWNNLRSQSKVSLPLDGSGDGPQSEFEAPSVSGISGSHKKRRI |
| | | | | (SEQ ID NO: 2) |
| cidB | WD0632 | nucle- | 3501 bp | GTGGATGGAGATCTTGATGGTTTTAGACAAGAGTTTGAATCCTTTT |
| | | otide | | TAGATCAATGTCCATTTTTCTTGTATCATGTAAGTACAGGACGTTT |
| | | | | CCTTCCTGTATTCTTTTTCAGTATGTTTGCTACTGCTCATGATGCTA |
| | | | | ATATCTTAAAAGCAAATGAGAGAGTGTATTTTCGTTTTGATAATCA |
| | | | | TGGTATTGATACAGGTGGTAGAAATAGAAATACAGGGAACCTAAA |
| | | | | AGTCGCTGTTTATCATGACGGACAGCAAGTTGTCAGATGCTACAGT |
| | | | | ATTTCTGATCGTCTTAATAGTGATGGGTTAAGGTTCAGTACAAGGG |
| | | | | AAAGAAATGCTCTAGTGCGAGAGATTAGAGGGCAAAATCCAAATT |
| | | | | TAAGGGAAGAAGACCTAAATTTTGAGCAATACAAAGTATGCATGC |
| | | | | ATGGAAAGGGCAAGAGTCAGGGAGAGGCGATTGCAACAGTATTC |
| | | | | GAGGTGATTCGTGAAAAAGATTCTCAAGGTAGAGATAGATTTGCT |
| | | | | AAATATTCAGCGTCTGAGATTAGCCTTCTTAGGCATATAGAACGCA |
| | | | | ATAGGCTTAATGGGATTAATGCGCCTGCGCCACGCAGTTTGTTGAC |
| | | | | AGTTAAGGAAATAGGAAGTATACGACTCAATCAAGATCAGAGAGT |
| | | | | ACAGCTTGGTCATTTGGTCAATTTTGTGCAAGTTGCACCGGGTCAG |
| | | | | CAAGGGATTTTCAGTTTTATGGAAGTGCTAGCAAGTAACCAAAAA |
| | | | | ATAAATATAGAACGTGGAATAAATGAAGGAATTTTGCCATACATA |
| | | | | ACTCGAATCTATCGTAGTTACCTAGGCAGCCTACAAAATGACATTC |
| | | | | AAAATCGCAGTCAAAAGTTTGAGAGTCACGGATTTTTCTTAGGTTT |
| | | | | GTTGGCAAATTTTATTCATCTCTACACAATAGATATTGACCTTGAC |
| | | | | TTGTCTCCTGGAAATTCATATGTTGCTTTTCTTATATGTCATCAGGC |
| | | | | AGAGAGAGAAAACATTCCTATCGTTATTAATGTTACTAGATGGAG |
| | | | | GACATCGTCTGATATTGCATTAAACCGCGCTAGAGCTGATGCTAA |
| | | | | AAGATTACATGTTTCTTCATTTATATCTATTCACACTGAATCAAGA |
| | | | | AATGCTGTTTGTATTGGATTAAATTTTAATCTGAATATAGATCCTTT |
| | | | | TAGTATTGATACAGTAGAGTTTTTAGAGAATAGATTTCCTTTGGTA |
| | | | | CAAAGATTATTTGAGTGTTTGGAGGATGAAGGAATTAGAGAAAAT |
| | | | | ATTAGAGATTTCTTGCTTCAACATCTTCCTAACGAAATACCAAGAA |
| | | | | ATGCAGAGAATTATAACAGAATATTTGATTGCATAACTGGTTTTGC |
| | | | | TTTTGGGAATAGTATTTTAGAAGAGTTCAGATTAGTAAACGCAGTT |
| | | | | CAACAACGTGTAAGAAAGTATATATTTAGATATGGTGATGAGAAT |
| | | | | CATGCTTTAACCATGGTCTTCCATACTCAAGGTTCTGATATAGTTA |
| | | | | TACTTCATATTAGAGATAACAACGCTGTACAACAAGGAGCCATCA |
| | | | | ATTTACAAGATCTTAATGTTGACGGAAATAATGTTCATGTACGGGA |
| | | | | AGTTTCATGCACACTTAATAATCAACTTGGCCTTAATATTCATACA |
| | | | | GATAACCTTGGTTTATATCACAATTACCAAAATAATAATGCAAATA |
| | | | | ATTTTCTTGGTGGTAATCTTGTGCAAGTGCCTAATGCTGGAAATGT |
| | | | | GCATAATGCTTTAAATCAAGTTATGAATGATGGCTGGCAAGATAG |
| | | | | ATTTCAGCATCAAGAATTATTTAGAAACATTTCTGCAGTATTAATG |
| | | | | CCAGAAGATACGCATGGCAATATGATAATAGATGTAAATAGCAAA |
| | | | | GATAAGTTTCGCTCTATACTACATGGTACATTTTATGCTAGTGATA |
| | | | | ATCCTTATAAAGTGCTTGCTATGTATAAAGTTGGTCAAACATATAG |
| | | | | TTTAAAAAGGTGGCAGGAAGAAGAAGGAGAAAGGGTAATACTTA |
| | | | | CAAGAGTTACAGAACAGAGACTAGGTCTTCTATTATTAAGACAAC |
| | | | | CTACAGCAGATACTCACCCAATTGGATATGTATTAGGATTTGCTGA |
| | | | | TAATGCAGAAGAAGTAGAACAGGAGCAAGACGAGGCAAGGTACA |
| | | | | AAATAACAGAATTGATGAGCAAACAAAGGGGATATTTGCCTATTA |
| | | | | CTTCTGGAAATGAGGTGGTTTTGTCTTATGCTGTATTTAATAGAGG |
| | | | | TGCACAGAGAGCAGAAGACTTTATATCTCTTCCACAACAAGCAGT |
| | | | | GTATGTACATAGACTTGATCGTCGTGGTCATGACTCAAGACCAGA |
| | | | | AGTATTAGTGGGACCTGAAAGTGTTATTGATGAAAATCCACCAGA |
| | | | | AAATCTATTGTCAGATCAAACTCGTGAAAATTTCAGGCGCTTTTAC |
| | | | | ATGGAAAAAAGACCAGGACAGAACTCGATTTTTTTGCTTGATATA |
| | | | | GATGATAATCTGCACGTTCCATTTAGTTACTTGCAAGGTACTAGAG |
| | | | | CACAGGCAATAGAAACATTAAGGTCAAGAATAAGGGGAGGTGGT |
| | | | | ACTTCTACAGCACAAGGAATATTACAACAAATAAACACTATCCTT |
| | | | | CGTAGAAACAACGCTCGTGAAATAGAAGATGTGCATAATCTACTT |
| | | | | GCACTAGACTTTGCAACAGAAAATCAAAATTTCCGTTATTGGCTAC |
| | | | | AAACTCATGACATGTTTTTCGCTGCACGACAATATACTTTCCATGA |
| | | | | TGATCGATCTAATCCAACTAATGATCGTCATGATTTTGCAATAACT |
| | | | | TCAGTAGGAGTCGATGGAAATCAAAATGATCCAACAGGTAGGGAC |
| | | | | TTATTAAGTAGTAACATAGATAACTTTAAACAAAAAGTAGATTCG |
| | | | | GGTGAAAAAGATAGATTAACTGCTATTATTAATGTAGGTAATCGT |
| | | | | CATTGGGTTACATTAGTTATTGTCCACCAAAATGGAAATTATTATG |
| | | | | GGTATTATGCTGATTCACTTGGTCCAGATAGTCGTATTGACAATAA |
| | | | | TATTCGAGGAGCTTTAAGAGAATGTGATATTAGCGATGATAATGT |
| | | | | CCATGATGTTTCCGTTCATCAGCAAACAGATGGCCATAATTGTGGC |
| | | | | ATATGGGCATACGAAAATGCTAGGGATATTAACCAAGCTATTGAT |
| | | | | CAAGCTTTACAGGGAAATAGTAACTTTGGAGAGAAAGGTGAAGGT |
| | | | | ATTATAGGTTATATACGTGGTCTTCTTAGTGCAGGAATTGGAAATG |
| | | | | ACACTAGACAACCTCAAAGAAATGAACAATACTTTAGAAATCGGA |
| | | | | GAAGAAATATTTCACAATTATTCCAAAATGATTCTCTATCTTCTCC |
| | | | | TAGGGGTAGATTGATTCAAGGTCGTCCAGGAATTCAACATGAAAT |
| | | | | TGATCCATTACTATTACAATTTTTAGAACTCCAATATCCACAGCGT |
| | | | | GGAGGTGGGGGAGCATTGCAATTAGGCGGAGAAAGAGTGATATC |
| | | | | AATTGATTTTGGTCCGCAATCTGTATTGGATGAAATTGATGGAGTG |
| | | | | AATAGAGTTTATGATCATAGCAATGGTAGAGGCAGTAGGTAG |
| | | | | (SEQ ID NO: 3) |
| cidB | WD0632 | amino | 1166 aa | MDGDLDGFRQEFESFLDQCPFFLYHVSTGRFLPVFFFSMFATAHDANI |
| | | acid | | LKANERVYFRFDNHGIDTGGRNRNTGNLKVAVYHDGQQVVRCYSIS |
| | | | | DRLNSDGLRFSTRERNALVREIRGQNPNLREEDLNFEQYKVCMHGKG |
| | | | | KSQGEAIATVFEVIREKDSQGRDRFAKYSASEISLLRHIERNRLNGINA |
| | | | | PAPRSLLTVKEIGSIRLNQDQRVQLGHLVNFVQVAPGQQGIFSFMEVL |
| | | | | ASNQKINIERGINEGILPYITRIYRSYLGSLQNDIQNRSQKFESHGFFLG |
| | | | | LLANFIHLYTIDIDLDLSPGNSYVAFLICHQAERENIPIVINVTRWRTSS |
| | | | | DIALNRARADAKRLHVSSFISIHTESRNAVCIGLNFNLNIDPFSIDTVEF |
| | | | | LENRFPLVQRLFECLEDEGIRENIRDFLLQHLPNEIPRNAENYNRIFDCI |
| | | | | TGFAFGNSILEEFRLVNAVQQRVRKYIFRYGDENHALTMVFHTQGSDI |
| | | | | VILHIRDNNAVQQGAINLQDLNVDGNNVHVREVSCTLNNQLGLNIHT |
| | | | | DNLGLYHNYQNNNANNFLGGNLVQVPNAGNVHNALNQVMNDGWQ |
| | | | | DRFQHQELFRNISAVLMPEDTHGNMIIDVNSKDKFRSILHGTFYASDN |
| | | | | PYKVLAMYKVGQTYSLKRWQEEEGERVILTRVTEQRLGLLLLRQPTA |
| | | | | DTHPIGYVLGFADNAEEVEQEQDEARYKITELMSKQRGYLPITSGNEV |
| | | | | VLSYAVFNRGAQRAEDFISLPQQAVYVHRLDRRGHDSRPEVLVGPES |
| | | | | VIDENPPENLLSDQTRENFRRFYMEKRPGQNSIFLLDIDDNLHVPFSYL |
| | | | | QGTRAQAIETLRSRIRGGGTSTAQGILQQINTILRRNNAREIEDVHNLL |
| | | | | ALDFATENQNFRYWLQTHDMFFAARQYTFHDDRSNPTNDRHDFAIT |
| | | | | SVGVDGNQNDPTGRDLLSSNIDNFKQKVDSGEKDRLTAIINVGNRHW |
| | | | | VTLVIVHQNGNYYGYYADSLGPDSRIDNNIRGALRECDISDDNVHDV |
| | | | | SVHQQTDGHNCGIWAYENARDINQAIDQALQGNSNFGEKGEGIIGYI |
| | | | | RGLLSAGIGNDTRQPQRNEQYFRNRRRNISQLFQNDSLSSPRGRLIQG |
| | | | | RPGIQHEIDPLLLQFLELQYPQRGGGGALQLGGERVISIDFGPQSVLDE |
| | | | | IDGVNRVYDHSNGRGSR (SEQ ID NO: 4) |
| cidA | WP0282 | nucle- | 1476 bp | ATGCCAACACAGAAAGAGCTTCGGGATACGATGTCCAAAAAATTA |
| | | otide | | CAGGAAGCTATTAAACATCCAGATCCAGCAGTTGTTGCCGGGAGG |
| | | | | AAGTCAGCTATCAAGAGATGGGTGGGAGTCCTTCAAGATAACTTT |
| | | | | ATGGAGCACATAAAATACTTTAAGGGTGATAAGTTGAAGTTTTTG |
| | | | | CACAATGTATTTCAAGATGAAGGTTGCTGGTCAGGTGTAAGGTTG |
| | | | | GATAATGCTGCTTTAGGTCAAAGGTTTACTGAAGAAAAAATAGGT |
| | | | | GGAATAGATAATCCACTTCGCAAATATGAGATGGCTTGTAGTTACT |
| | | | | GTGTGGTGGATAAAATTCATCCTCTCTTTCAAAAAAGATTTGAATC |
| | | | | TTATAGGAACAAGTTTCCTCCTGGTGCATTTGATGGTAAAACTGAA |
| | | | | ACTGAATTTGGCAAATACGTACGAAACTCGTTACTAGATAGCATA |
| | | | | AAGAGGAAAGGTCCTGTATTTGATTTCTGGATTGATAGAGAATCT |
| | | | | GGGGAATTAAAGAAGTATGATGCAGTAGAAGGTTTTGACAGTGCT |
| | | | | GTAAAATTTAAGTGGAGTGAAGGGGTAGAGTATTTTTATAATCATT |
| | | | | TAAAAGAAGAAGATAAGGAAAAGAAGCTCACAGAAGCTATTCTT |
| | | | | GCTCTTTCTCGCGTTCAATCTGTTGAGAAAGACGCCCCTATTTTAG |
| | | | | ATTTTTGTGTAAATAAGATAGTCGATAAAGATACTCTTTTACAGAA |
| | | | | ATTATCACAGAAAGATAAAGGAGTATATTCCCTTTTTGCTGAATTA |
| | | | | ATAGAGTCATGTTTTTTTGATACGGTTCATGATTTGGTACAGTGCT |
| | | | | GGTGTTATAAAGAAGTTTCAGCAGGAGGAGACCATTCAGAGAAAA |
| | | | | TATTCTCACAGCGAGACTATGAGCTTTTTCTTTCCTCTCTTTCAGAC |
| | | | | ACAATGTTGAAAAATCCTGAGTTAAGCGTTCAAGCTAGATCTCTTA |
| | | | | TTATGGAATTTTGGGAATGTGGTAGCTTGTATCAATACAGAAAAG |
| | | | | CTGCTGTTAATACTTCTAATTATACAGTTCCTACAAGTGGTGTATTT |
| | | | | GCAGAGTTAATAGTCAATTGGAGACGAGAAGACATTTATAAGACT |
| | | | | GATGAAGAAAAAGAAATAGAGAAAAAAGAAATATTAGATATGAT |
| | | | | GTCATTTGCCAAAGATTGCTTTCCTGAAAAGTTTGAGCTCTTTAAA |
| | | | | AAACTAATAATAAGAGACCTTAGATTATGCGGTAGGGAAGGTAAA |
| | | | | AGAGTAAATGTAGATTACGGTCTGTTTGCAGAAGAATTATTCTCTG |
| | | | | AGTTAGAGAAAACAATTTTACCACCTGGTCCTGTAGGTGATGGTCC |
| | | | | TTGCAGTAATTTGCGATCACGATCTAAAGCTCATGGTAGTAAGAA |
| | | | | AACAACTTTGCCAGTTGATGATAGTCCGCAGTCTGAGCTTGGAACT |
| | | | | CCTAGTGTAAGTGGTGTTTCTTCTTATAAGAAAAAAAGCGTCTTTA |
| | | | | CGCTTAGTGGTAATAAGTAA (SEQ ID NO: 5) |
| cidA | WP0282 | amino | 491 aa | MPTQKELRDTMSKKLQEAIKHPDPAVVAGRKSAIKRWVGVLQDNFM |
| | | acid | | EHIKYFKGDKLKFLHNVFQDEGCWSGVRLDNAALGQRFTEEKIGGID |
| | | | | NPLRKYEMACSYCVVDKIHPLFQKRFESYRNKFPPGAFDGKTETEFG |
| | | | | KYVRNSLLDSIKRKGPVFDFWIDRESGELKKYDAVEGFDSAVKFKWS |
| | | | | EGVEYFYNHLKEEDKEKKLTEAILALSRVQSVEKDAPILDFCVNKIVD |
| | | | | KDTLLQKLSQKDKGVYSLFAELIESCFFDTVHDLVQCWCYKEVSAGG |
| | | | | DHSEKIFSQRDYELFLSSLSDTMLKNPELSVQARSLIMEFWECGSLYQ |
| | | | | YRKAAVNTSNYTVPTSGVFAELIVNWRREDIYKTDEEKEIEKKEILDM |
| | | | | MSFAKDCFPEKFELFKKLIIRDLRLCGREGKRVNVDYGLFAEELFSEL |
| | | | | EKTILPPGPVGDGPCSNLRSRSKAHGSKKTTLPVDDSPQSELGTPSVSG |
| | | | | VSSYKKKSVFTLSGNK (SEQ ID NO: 6) |
| cidB | WP0283 | nucle- | 3525 bp | ATGAGTAATGGTGATGGACTTATTAGGAGTTTGGTGGATGGAGAT |
| | | otide | | CTTGAAGGATTCAGACAAGGATTTGAATCTTTTTTAGATCAATGTC |
| | | | | CATCTTTCTTGTATCATGTAAGTGCAGGTCGTTTCCTTCCTGTATTC |
| | | | | TTTTTTAGTATGTTTTCTACTGCACATGATGCTAATATCTTAAATGC |
| | | | | AAATGAGAGAGTCTATTTTCGTTTTGATAACCATGGTGTTAATCCA |
| | | | | CGTAATGGTGAAAATCGAAATACGGCAAACCTAAAAGTTGCTGTT |
| | | | | TATCGTGACGGACAGCAAGTTGTCAGATGCTACAGTATTTCTGATC |
| | | | | GTCCTAATAGTGATGGGTTGAGGTTCAGTACAAGGGAGAGAAATG |
| | | | | CTCTAGTACAAGAGATTAGACGGCAAAATCCAAATTTAAGGGAAG |
| | | | | AAGACCTAAATTTTGAGCAATACAAAGTATGCATGCACGGAAAGG |
| | | | | GCAAGAGTCAGGGAGAGGCAATTGCAACGGTATTCGAGGTAATTC |
| | | | | GTGAAAAAGATCGTCAAGGTAGGGATAAATTTGCCAAATATTCAG |
| | | | | CATCTGAGGTTCATTTCTTGAGGCAACTCTTTAGAAATCACAGATT |
| | | | | AACAATTAAGGAAATAGAAGGAAGACAACTCAATCAAAATCAGC |
| | | | | TCAGACAACTTGGTAGGTCAGTCAATTTTACACGAGTAGAACCAG |
| | | | | GTCAGCAGAGGATTGACAACTTTATGGAAATGCTAGCAAGTAACC |
| | | | | AAAGACAAGATGTAAGGGATTCTCTCCGAGGAGATATTTTAGAAT |
| | | | | ATGTAACTGATACCTATAACAATTATAGGGCACAGATAGAAAATA |
| | | | | ATATTGAAGGTCGCAGTCAAAAGTTTGAGAGTCATGGGTTTTTATT |
| | | | | AGGTTTCTTAGCAAATTTTAGTCATCGCTACACAATAGGCGTCGAT |
| | | | | CTTGACTTATCTCCTAGAAACTCACATGTTGCATTTCTTGTACGTCA |
| | | | | TCAAGTAGAAAGAGAAAATATTCCTATTGTTATTAATCTTGCTACA |
| | | | | AGGGCACCGCCCTATATCGCATTAAACCGCGCCAGAAGTCACGCT |
| | | | | GAAAGATTGCATGTTTTTTCATTTATACCTATCCATACTGAATCAA |
| | | | | GAAATACTGTCTGTGTTGGATTAAATTTTAATTTAAATCTAGATCC |
| | | | | TTTTAGTGTTGATACAGTAGGGCTTCAACAGGATAGATTTCCTTTA |
| | | | | GTACAAAGATTATTTGAGTGTTTGGAGAATGAAGGAATTAGAGAA |
| | | | | AATATTAGAGATTTCTTGCTTCACCATCTTCCTGCTGAAATACCAA |
| | | | | GAAATGCAGAGAATTATGATAGAATATTTGATTGCATAACTGGTTT |
| | | | | TGCTTTTGGGAATAGTGCTTTTGATAGGCACCCTTTAGAACTAGAA |
| | | | | GAGGAAGACGAAGCACCTATAACAAAGTACATATTTAGACATGGT |
| | | | | GATGAGGGTTTAAGATGTTTAACTATGGTCTTTCATGCTGAAGGTT |
| | | | | CTGATATAGTTATACTTCATATTAGAGCTCACGATGCGCAACAACA |
| | | | | AGGAGCCATCAATTTACAGACTCTTAATGTTAATGGAAATGATGTT |
| | | | | CATGTGTGGGAAGTTTCATGCACACTTAATAATCAACTTGAACTAG |
| | | | | ATATTGATCTACCAAATGACCTTGGTTTATATCACGATTACCAAAA |
| | | | | TAATAATGCAAATAATTTTCTTGCTGGTGATCTTGTACAAGTGCCC |
| | | | | AATACTGAAAATGTACATAATACTTTAAATCAAGTTGTGAATGAT |
| | | | | GGCTGGAAAAATATAGCTCAGCATAGAGGATTATTTCAAGAGATC |
| | | | | TCTGGAGCATTGATGCCGCTTGTGGATACAATAAATGTTAATAGTG |
| | | | | AGGATAAGTTCCGTTCTATACTACATGGTACATTTTATGCTAGTGA |
| | | | | TAATCCTTATAAAGTGCTTGCTATGTATAAAGTTGGTCAAACATAT |
| | | | | AGTTTAAAAAGGGGGCAGGAAGAAGAAGGAGAAAGGGTAATACT |
| | | | | CACAAGAATTACAGAACAGAGATTAGATCTTTTATTATTAAGACA |
| | | | | ACCTAGAGAGAATGACCTAGATACTCACCCAATTGGATATGTGTT |
| | | | | AAGACTTGCTAATAATGCAGAAGAAGTAGGACAACAGCAAAATG |
| | | | | ATGCGAGACAAGAAATCGGAAGACTTAAGAAACAACACAGAGGA |
| | | | | TTTATACCTATTACTTCTGGAAATGAGGTGGTTTTGTTTCCTATTGT |
| | | | | GTTTAATAGAGATGCACACGAAGCAGGTAATCTTATACTTTTCCCA |
| | | | | GAAGGGATAGGAAGAGAAGAGCATGTACACAGGCTTGATCGTCAT |
| | | | | GTTCGCAGCTCAAGACCAGGAGGATTAGTGGGACCTGAAAGTGTT |
| | | | | ATTGATGAAAATCCACCAGAAGGTCTATTATCAGATCAGACTCGT |
| | | | | GAAAACTTTAGGCGTTTTTACGAAGAAAAAGCACCAGGACAAAAT |
| | | | | TCGATTTTTTTGCTTGATATAGGCGACAATCTACATGTTCCCTTTAG |
| | | | | TTACTTGCAAGGTACTAGAGCACAGGTAATAGAAACATTAAAGTC |
| | | | | AAGAATAAGGGGAGGTGGTACTCCTACAGCACAAGGAATATTACA |
| | | | | ACAAATAAATGCTATCCTTCGTAGAAACAACGCTCGTGAGATAGA |
| | | | | AGATGTGCATGATCTACTTGCACTAGACTTTGCAACAGATAATCAA |
| | | | | AATTATCGTTATTGGCTACAAACTCATGACATGTTTTTCGCTGCAC |
| | | | | GACAATATACTTTCCTTGATAATCAATCTCATTCAACTAATGATCA |
| | | | | TTATGGTTTTGAAATAACTTCAGTAGGAGTCGATGGAAATCAAAA |
| | | | | TGATCCAACAGGTAGGGGCTTATTAAGTAGTCACATAACTAACTTT |
| | | | | AAACAAAAAGTAGATTCGGGTGAAAAAGATAGATTAATTGCTATT |
| | | | | ATTAATGTAGGTAATCGTCATTGGGTTACATTAGTTATTGTACACC |
| | | | | AAAATGGAAATTATTATGGGTATTATGCTGATTCACTTGGTCCAGA |
| | | | | TAGTGGTATTGACAATAATATTCGAGGAGCTTTAAGAGAATGTGA |
| | | | | TATTAACGATGATAATGTCCATAATATTTCCGTTCATCAGCAAACA |
| | | | | GATGGCCATAATTGTGGCATATGGGTATACGAAAATGCTAGGGAT |
| | | | | ATTAACCAAGCTATTGATCAAGCTTTACAGGGAAATAATAACTTTG |
| | | | | GAGAGAAAGGTGAAGGTATTATAGGTTATATACGTGGTCTTCTTA |
| | | | | GTGCAGGCATTGGAAATGACACTAGACAACCTCGAAGAAATGAAC |
| | | | | AATACTTTGAAGATCGGAGAAGAGATATTTCACAATTACTCCAAA |
| | | | | ATGATCCTAACTTACCTTCTCGCCGGAGTGATTTAATTCAAGCTCA |
| | | | | TCCAGGAATTCAACATGAAATTGATCCATTACTATTACAATTTTTA |
| | | | | GGACTCCAATACCCACAGCGTGGAGGTGGAGGAGCATTACAATTA |
| | | | | GGCGGAGAAAGAGTGATATCAATTGATTTTGGTAACCCGCAGTCT |
| | | | | GCATTAGATAAAATTGATGGAGTGAGTAGAGTTTATAACCATAGC |
| | | | | AATAGTAGAGGTAGTAGGTAG (SEQ ID NO: 7) |
| cidB | WP0283 | amino | 1174 aa | MSNGDGLIRSLVDGDLEGFRQGFESFLDQCPSFLYHVSAGRFLPVFFF |
| | | acid | | SMFSTAHDANILNANERVYFRFDNHGVNPRNGENRNTANLKVAVYR |
| | | | | DGQQVVRCYSISDRPNSDGLRFSTRERNALVQEIRRQNPNLREEDLNF |
| | | | | EQYKVCMHGKGKSQGEAIATVFEVIREKDRQGRDKFAKYSASEVHF |
| | | | | LRQLFRNHRLTIKEIEGRQLNQNQLRQLGRSVNFTRVEPGQQRIDNFM |
| | | | | EMLASNQRQDVRDSLRGDILEYVTDTYNNYRAQIENNIEGRSQKFES |
| | | | | HGFLLGFLANFSHRYTIGVDLDLSPRNSHVAFLVRHQVERENIPIVINL |
| | | | | ATRAPPYIALNRARSHAERLHVFSFIPIHTESRNTVCVGLNFNLNLDPF |
| | | | | SVDTVGLQQDRFPLVQRLFECLENEGIRENIRDFLLHHLPAEIPRNAEN |
| | | | | YDRIFDCITGFAFGNSAFDRHPLELEEEDEAPITKYIFRHGDEGLRCLT |
| | | | | MVFHAEGSDIVILHIRAHDAQQQGAINLQTLNVNGNDVHVWEVSCTL |
| | | | | NNQLELDIDLPNDLGLYHDYQNNNANNFLAGDLVQVPNTENVHNTL |
| | | | | NQVVNDGWKNIAQHRGLFQEISGALMPLVDTINVNSEDKFRSILHGT |
| | | | | FYASDNPYKVLAMYKVGQTYSLKRGQEEEGERVILTRITEQRLDLLL |
| | | | | LRQPRENDLDTHPIGYVLRLANNAEEVGQQQNDARQEIGRLKKQHR |
| | | | | GFIPITSGNEVVLFPIVFNRDAHEAGNLILFPEGIGREEHVHRLDRHVRS |
| | | | | SRPGGLVGPESVIDENPPEGLLSDQTRENFRRFYEEKAPGQNSIFLLDI |
| | | | | GDNLHVPFSYLQGTRAQVIETLKSRIRGGGTPTAQGILQQINAILRRNN |
| | | | | AREIEDVHDLLALDFATDNQNYRYWLQTHDMFFAARQYEFLDNQSH |
| | | | | STNDHYGEEITSVGVDGNQNDPTGRGLLSSHITNFKQKVDSGEKDRLI |
| | | | | AIINVGNRHWVTLVIVHQNGNYYGYYADSLGPDSGIDNNIRGALREC |
| | | | | DINDDNVHNISVHQQTDGHNCGIWVYENARDINQAIDQALQGNNNF |
| | | | | GEKGEGIIGYIRGLLSAGIGNDTRQPRRNEQYFEDRRRDISQLLQNDPN |
| | | | | LPSRRSDLIQAHPGIQHEIDPLLLQFLGLQYPQRGGGGALQLGGERVIS |
| | | | | IDFGNPQSALDKIDGVSRVYNHSNSRGSR (SEQ ID NO: 8) |
| cixA | wRi_06720 | nucle- | 1371 bp | ATGCCAAAAAAGATGGAGCGTCATGCTGCAGTGCTTAGTAAGTTA |
| | | otide | | AAGAGTGTTATTCAACATACAGATTCCAAGGTCATGGCTGAAAGG |
| | | | | CGTTCAGCTATTGAAAGATGGGTAAAAACTTACATTAGGCAGGTA |
| | | | | GAATATCTTAAAGATGATAAGCTACAATTCTTATACAACATATTTC |
| | | | | GCGATGAAAGTTGTTGGTCAGGTACGAGATTGAACAATACAATCT |
| | | | | TAGGACAGAGGTTTACTGAAGAAAAAATAGGCGAAATAAAGAAC |
| | | | | CCTCTTCCTATATATGATATGGCATGTCGATACTGCGTGATAGATA |
| | | | | AAATTCCTTTGCTCTTTCAGAAGCAGTTTGAATCTTACAAAAGTAG |
| | | | | CTTCTCTTCTGAAGAGATAGATGATGATGGTAAGCCTGCAACTAGC |
| | | | | AATAACAAATATGTAAAGAGTGAGTTGTTGGGTTATATGAAGAGT |
| | | | | CAAGACCCTGTATTTAGCTTTTGGGTTGATAAAAAATCTGGAGAAT |
| | | | | TTAAGAAGCATGTCAGCGCAACAGAAGGATTTAAGAAAGCTATAG |
| | | | | AACTTAAGTGGAGCGAAGGAGTAGAATATTTTTATAGCCTTCTAA |
| | | | | ATGAAAAAGAAAGAGAAAGAGAAAGGAAAATTACTGATGCAGTT |
| | | | | ACTATATTATCCTCTGTTCAATGTGACCATAATGGTGCTGTTACTTT |
| | | | | AGACTTTTGTCTTAGTAAAATGAGCGATCAAGCAAAAAACAAGCT |
| | | | | GTTTAAAGATTCTGAGCTATCAAAAAAAGATAAAGGAGTGTACTC |
| | | | | TCTCTTTAGCGCGTTGATACATCAAGGTTTTTTTGATACGATGCAA |
| | | | | GCTATACTTCCGATGTTTAAAGATAAAATACTGGAGGATAAGATA |
| | | | | CTTTCACCTAGGAGTTATACTCTTCTTCTCTCCTCACTTTCGGACAT |
| | | | | GATGCTCGAAAATTCTGAGTCAACTATTCAAGCTAGGGAAGCTAT |
| | | | | AATGAACCTTATAAAGTGTGGTAATTTCAATAATCATGAGGGGCG |
| | | | | TGAGGAAAAAGCTGCGGTATTTTTTTCTAATGGAAGGGTTCCGATT |
| | | | | AAGCGTGCGCTTGCAGGATTGATTGTCGATTGGCAACTTGGTTGTA |
| | | | | CAAAAAAGGAAGAGGTGTTAAAGGTACTACAGTTTGCCAAAGAGT |
| | | | | TTTGTGCAGTTGAAAGTTTTATGTATTTTAAAAAATCTGTTGTTGAT |
| | | | | AACCTAAAAATGGTTGGTAGGGATGGTATGAGAAAAAATATAGAC |
| | | | | TATGGTAAATTAGCAGAAAAGTTGTTTGCTGAATTAGATACGGTAT |
| | | | | CCGTGCCTAACGGAAGAGGTGATTTTGGTGGAGCTGGTGACCCAC |
| | | | | AGTCTACACTAGGAAGCACTGAAGTTAGTAGTTTTTCTGGTCGCAA |
| | | | | TAAGTAG (SEQ ID NO: 9) |
| cixA | wRi_06720 | amino | 456 aa | MPKKMERHAAVLSKLKSVIQHTDSKVMAERRSAIERWVKTYIRQVE |
| | | acid | | YLKDDKLQFLYNIFRDESCWSGTRLNNTILGQRFTEEKIGEIKNPLPIY |
| | | | | DMACRYCVIDKIPLLFQKQEESYKSSFSSEEIDDDGKPATSNNKYVKS |
| | | | | ELLGYMKSQDPVFSFWVDKKSGEFKKHVSATEGFKKAIELKWSEGV |
| | | | | EYFYSLLNEKERERERKITDAVTILSSVQCDHNGAVTLDFCLSKMSDQ |
| | | | | AKNKLFKDSELSKKDKGVYSLFSALIHQGFFDTMQAILPMFKDKILED |
| | | | | KILSPRSYTLLLSSLSDMMLENSESTIQAREAIMNLIKCGNFNNHEGRE |
| | | | | EKAAVFFSNGRVPIKRALAGLIVDWQLGCTKKEEVLKVLQFAKEFCA |
| | | | | VESFMYFKKSVVDNLKMVGRDGMRKNIDYGKLAEKLFAELDTVSVP |
| | | | | NGRGDFGGAGDPQSTLGSTEVSSFSGRNK (SEQ ID NO: 10) |
| cixB | wRi_06710 | nucle- | 2265 bp | ATGCATGGGTTAGTTAGAAGTTTAATAAATGGAAATTGTGGAGAA |
| | | otide | | TTCACGGAAAAGTTTGAATATTTCTTGGATTCATGTCCATCTTTTCT |
| | | | | GCATTCAGTTGGCAAAGATCACTTTTTTCCTGCGTTCTTTTTTGGCA |
| | | | | TGTTTGCTACTGCACATGATTCTGGTGTTGCAAACAATGATGAAAG |
| | | | | AATCTTCTTTCGTTTTGATAATGATCCAGGTAGTCCTGGAAGGGGA |
| | | | | AATCTAAAGGTTGCAATTCTAACAACTGATGGAAATAACAGAAGA |
| | | | | GTTGTAAGGTGCTATACTATTGCTGACAGAGAGAATAGCTACGGT |
| | | | | TCTAGGTTTAGCCAGCAGGAAAGGGAGCAGCTGGAAGGTATCCTG |
| | | | | CGAGATGAAGAGCTTGAATGGCAAGAGTATAAAACATTTATATGG |
| | | | | GCGGATAATCAAGGTGAAGATGAAGAAGAGGAAGCAGTAAGATG |
| | | | | TAGGATATTTCAGGCAGGACAAGGGCCGTTTACTGGAAATCATGC |
| | | | | ATCTTATTTAACTCGTAGACATAGTTTTCAAGAGATTACCAGAACA |
| | | | | CCTGGGCTGCAAAATAATTATTTACCGGATTTGATGAATCAGCTAG |
| | | | | AAAGTGATGATGCAGATGATGTACACGACACTACTGAGGAAGTGT |
| | | | | TTCAGCATATTATTGGTGTCTACGATAGATATAGTCAGGCATTGGA |
| | | | | CTTCTATGGTAGAGAGTCTGACTATCATGGTTTTGTTTCCGGTGTTT |
| | | | | TGATGCATTTTAGATATCGCAATGTAGCCAATATTTACCTTGAGCT |
| | | | | GTTTGTAGGTGGTGGATATGCAGATATTACTTCTATTGTACGTGGT |
| | | | | ACACAGAGGTTAATTAATTCTGTTCCCTGTGTAACTGAACTTAAGG |
| | | | | CAGGCAGAAGAGCAGATAGGAATGCTGGCCGTGCATTAGAGCAG |
| | | | | GCTGGAAATTATGTTAATGGATGTCCCGTTTCATCCATATCTATTC |
| | | | | CAACATTATCACCAAGAGCTGTCTCCGCTGGAGTGAATTTCGATTT |
| | | | | TGGTAACCCAGGACGTTTACAGCTTGGTGTGAGGGCTTTTTTAGCA |
| | | | | AAAGGTTCTTCTTTAATGGAAAGATTATTTGAACCTGTAGAGGATG |
| | | | | AGGAGATTGGAGAAAATGTTAGGGATTATCTACTCCATCCAGCCT |
| | | | | TTGGTGTACCTGCTGTACCAGGTATTAGGAATAGGGGTGGTGTTAA |
| | | | | CGCTAGAGATAGAAGAATATTTCTCTATACAAGTGGATTTGCTTTC |
| | | | | GCAAGTATTGCATTTGCAAAAGGAACTGTGCCAATAGAAGGAAAT |
| | | | | CGTGCAATAGTAGATAAGCACTTGTTTCACTATGACGGTAATGCA |
| | | | | AAAATGTTAGATGAGCAAAGATACAATACACAAGTAAATATTGGA |
| | | | | GATCGTGCTTTGACTATGGTTTTGCATGTATCACGAGGTAGAGACC |
| | | | | AGAAGGAGGAGGTGATCGTATTTCATGTTCGCCACGTATTGGCTA |
| | | | | ATCAACTTTTTCCGGACAATGGATTGGATCTATCGCGTTGGCCGAA |
| | | | | TGCTATGGTACATGAAGTGGTGTGTAATTTGACCATAAATAGAAG |
| | | | | GACAAGAGGAGTAAATGATAATCTTGGTTTAACTGTTAATGTAGA |
| | | | | AACATTTGACTCGCCTGCTGACTACCTGCTTGATAGAGGTAATCAG |
| | | | | CCTTTTCAAGGTGAGCTTTTACGAATAGGTGGCGTTAGTAATGTGC |
| | | | | ATCGCGCTGCAAATGTAATGATGAATACTGGCTGGGAAAATGAAG |
| | | | | ATCCAGACAGTCATGAACGGTTTTACCAAGCAATTTCCAACGTGCT |
| | | | | AAATCCACCCCAGCCAAATAATGCAGGACTCCAATCATTAGCATG |
| | | | | GGTAGTGAACAGAGATAATGCTAGAGAAGCTGGGTTTCATGCTGC |
| | | | | ATTGCATGGATTATTTTACACTTGCGATAATCCTGCTAGGGTAGTT |
| | | | | AGTGAATTTCAGGTTGGAGGAGGAGGAAAGTTAGACTTAGTATTG |
| | | | | TCACGAGCTATAGGAAGGATGGGAGGTACTTATCCGATTGGAACA |
| | | | | GAGCTAAAGTTTGCTGCCACTGAAGCAGACGTACAAAATAGAGAA |
| | | | | GAAGAAGCAGATGAACAGGTGGAGGGTTATCTGCAGAGTAGAGG |
| | | | | GTTTGATCGCATTACTGATGGAGATAAAATGGTTTTCTCGTATGCC |
| | | | | GTATTTAATGATCAAGCGCCAGCACCAGCACAAAATGTCCCAAAT |
| | | | | ACCCTTATAGCAGTTAGTAATGTTCTACGCATAAAAGATAACTTAG |
| | | | | GAATTGACACTGTGGACGACTTTCCTTATAGATAA (SEQ ID NO: 11) |
| cixB | wRi_06710 | amino | 754 aa | MHGLVRSLINGNCGEFTEKFEYFLDSCPSFLHSVGKDHFFPAFFFGMF |
| | | acid | | ATAHDSGVANNDERIFFRFDNDPGSPGRGNLKVAILTTDGNNRRVVR |
| | | | | CYTIADRENSYGSRFSQQEREQLEGILRDEELEWQEYKTFIWADNQGE |
| | | | | DEEEEAVRCRIFQAGQGPFTGNHASYLTRRHSFQEITRTPGLQNNYLP |
| | | | | DLMNQLESDDADDVHDTTEEVFQHIIGVYDRYSQALDFYGRESDYH |
| | | | | GFVSGVLMHFRYRNVANIYLELFVGGGYADITSIVRGTQRLINSVPCV |
| | | | | TELKAGRRADRNAGRALEQAGNYVNGCPVSSISIPTLSPRAVSAGVNF |
| | | | | DFGNPGRLQLGVRAFLAKGSSLMERLFEPVEDEEIGENVRDYLLHPAF |
| | | | | GVPAVPGIRNRGGVNARDRRIFLYTSGFAFASIAFAKGTVPIEGNRAIV |
| | | | | DKHLFHYDGNAKMLDEQRYNTQVNIGDRALTMVLHVSRGRDQKEE |
| | | | | VIVFHVRHVLANQLFPDNGLDLSRWPNAMVHEVVCNLTINRRTRGV |
| | | | | NDNLGLTVNVEITDSPADYLLDRGNQPFQGELLRIGGVSNVHRAANV |
| | | | | MMNTGWENEDPDSHERFYQAISNVLNPPQPNNAGLQSLAWVVNRD |
| | | | | NAREAGFHAALHGLFYTCDNPARVVSEFQVGGGGKLDLVLSRAIGR |
| | | | | MGGTYPIGTELKFAATEADVQNREEEADEQVEGYLQSRGFDRITDGD |
| | | | | KMVFSYAVFNDQAPAPAQNVPNTLIAVSNVLRIKDNLGIDTVDDFPY |
| | | | | R (SEQ ID NO: 12) |
| cinA | wNo_01990 | nucle- | 1473 bp | ATGCCAAAAAGTAAAACTAAACGTGGAACGGAAGATTTGAAGGG |
| | | otide | | TAATGCAGGCCCAAGCAAAAGATCTCGTCTCAGTTCTGATCCTAA |
| | | | | AAAAAATAAAGAGATTATCTCTAGCAAAGTAATAAGTAAGCTGAA |
| | | | | GGATGTTGTTAAAGGTGATAGAACTTCAGCTATTGAGGAATGGGT |
| | | | | CAAGGCTCACCCTGTCACAGTAGAGGGTCTAATCGTTGAGCAATC |
| | | | | GGACCTCTTATGTAATGCGTTTCGTGATGAATCTTGTTGGTCAGGT |
| | | | | GCGACACTAGATGTTGCTAAATTGGTAGGAGAATTAGCTAAATCA |
| | | | | GGTGTGTTGAATCCATTTGCTATATATAAAATAGCATGTATTGAGT |
| | | | | GTGTAGAGAGTGAAATTAAGCAATTATTTGACAAGGCGTTAGAGT |
| | | | | CTTTTAGATCTGACTTATCTCATAAAGGTGCATGTGAGGAAGATAG |
| | | | | GAATTTAGCTTGCAGTGATAAGCTTGCAAGAGTTGAATTGTTAAGT |
| | | | | TCCATGGGAAGACGTGATCCTGTTTTCAATTTCTGGATTGATCAAG |
| | | | | AATCAGGTAACCTTAGAGAAAATATAGAAGCAGAAGATGGATTTA |
| | | | | ATAAGGCTGTAGATTTCAAGTGGAGTAAGGGAGTGGAACACTTCT |
| | | | | ATAATCGTCTGTGTTCTGAAGAAAAATTAGTGAAAGAAGAGAGAG |
| | | | | AAAAATTGCTAGTTTCTGCTATTGCAAAATTATCTCCATTGCAATC |
| | | | | TAGCTATAAACTTGCTTCTACCTTAAATTCCCTTCTAGGTAAAGTC |
| | | | | ATAAGCGCAAAAGTAGATCATAAGTCACTACTTGGGCTACCGAAT |
| | | | | AAGAGAGATAGGGGTGTGATCTATCGTCCTCTTAGTTACTTAGTAG |
| | | | | AGCACGGTTTTCTTTGCACAACTAAGTATGTTATCCAGTACTTGAG |
| | | | | CGAGGGATGTTCAAGATCTGAAGTAGAGAAAATGCTTTCACCTAG |
| | | | | AGGATATGCACATCTTCTCTCATCGCTTTCATTTGTTGTAGTTTCTA |
| | | | | AAGATTATGACTTGGATAACAGGAATGAAGCAAGGTCAGCTATTA |
| | | | | GCAGTCTTTGGGAATCTAGTGTATTTAACCAAAATAAAATAAATGT |
| | | | | TGTCGATCCTTTTAAAGATAGGATTGCTTTTGTTGCAATGGAAAAT |
| | | | | GCAATTTCAAATTTGATTGTAGATCAGGAGAACAGTAAGGATACT |
| | | | | CAAAGTGCTGGCGATGGTGAAAAAGTTGATTTGGTCTTGAGTATTT |
| | | | | TAAAGTTTGCTAAAGATTGTTGTTCAGACAAAAGCTTTAAATCATT |
| | | | | AAAAGCGAGGATAGCAAATAGTTTAGATAAAACAAGGAATTCTAA |
| | | | | GATGATAGATGCAACTAGCTCCTGCAATTTAATAGAAGAGTTGTG |
| | | | | TAAGTCAGCGAGAAATTTGAATTTATTCTCTGCTAGCACTGAAGGT |
| | | | | CCTCAATCTACGTTAGTGGGTACTAATGTTAGTATTTCGCCTGCTG |
| | | | | CAGTTGTTAACAAATAG (SEQ ID NO: 13) |
| cinA | wNo_01990 | amino | 490 aa | MPKSKTKRGTEDLKGNAGPSKRSRLSSDPKKNKEIISSKVISKLKDVV |
| | | acid | | KGDRTSAIEEWVKAHPVTVEGLIVEQSDLLCNAFRDESCWSGATLDV |
| | | | | AKLVGELAKSGVLNPFAIYKIACIECVESEIKQLFDKALESFRSDLSHK |
| | | | | GACEEDRNLACSDKLARVELLSSMGRRDPVFNFWIDQESGNLRENIE |
| | | | | AEDGFNKAVDFKWSKGVEHFYNRLCSEEKLVKEEREKLLVSAIAKLS |
| | | | | PLQSSYKLASTLNSLLGKVISAKVDHKSLLGLPNKRDRGVIYRPLSYL |
| | | | | VEHGFLCTTKYVIQYLSEGCSRSEVEKMLSPRGYAHLLSSLSFVVVSK |
| | | | | DYDLDNRNEARSAISSLWESSVFNQNKINVVDPFKDRIAFVAMENAIS |
| | | | | NLIVDQENSKDTQSAGDGEKVDLVLSILKFAKDCCSDKSFKSLKARIA |
| | | | | NSLDKTRNSKMIDATSSCNLIEELCKSARNLNLFSASTEGPQSTLVGT |
| | | | | NVSISPAAVVNK (SEQ ID NO: 14) |
| cinB | wNo_01980 | nucle- | 2091 bp | ATGCATGGTAATAATGAAGATCGTGAATTAGTTAGGGCTTTATTAA |
| | | otide | | GTGGAGGTTGTGATGAGTTTAGTAGACAATTTGTAGGTTTTTTAAA |
| | | | | CAACTGTCCATCTTTTTTGCATTCGGCTAATAAGCCTGGCTTTTTTC |
| | | | | CTACATTCTTTTTTGGTATGTTTTCTACTGCACATGATGCAGGTATA |
| | | | | TTAGTTGAAGGTGAAAGAGTCTATTTTCGTTTTGACAATTATGGAA |
| | | | | ATCTAAAAGTTGCTGTTCTCACTAATAAAGAAAATAGAAGAATAG |
| | | | | TCAGGTGTTATACTGTTGCTGATAATGAGAACAGCCCTGGGTCAA |
| | | | | GGTTTAGTGCAGAAGAGAAGCAGCAGGTAGAAGAGAATCTTCCAC |
| | | | | AAGAATTACAGGAAGATGAGGATCTGGATTGGGAAGAGTATAAA |
| | | | | ATATTTCGGTTTGGAGAAGAATGTAGGTTTATTCATGAAATAGATA |
| | | | | GATTTCCTCAACGTGATGAACCTGGAGCTCCAATTTTTCATGAAAT |
| | | | | TAACCCAATCAGAGAACAAGGTGAATTGTTAGACCTGATGAGTGA |
| | | | | GTTGGCAAATGACGATACAGGAGAAGTGCGTACTAATGTTAAAAG |
| | | | | AATTTTGGAATATGTTATTGATATCCATGATGAACATGAAGATAGC |
| | | | | TTAGTGTTTCGTGCAGAGTCTGACTACCACGGTTTTCTGTGTGGGT |
| | | | | TTTTAGTAAATTTTAGATACCGAGCTTTGGCTGATTTCTACCCAGA |
| | | | | GCTACTTATAGGAAAAGGTTATGCAGATGTTGTTTTGCTTGTTCGT |
| | | | | GGTGTTGATCAGACAAATGATTCGGTTCCAATTATAATTGAGTTGA |
| | | | | AGGTTGGTGATGAGGAAGGATTAGAGCAAGCTAAAGATTATGCTA |
| | | | | AAAGTTGTTCTGTTTCGTCTTTGCCTATTCATACCTCATCACCAAGT |
| | | | | GCTGTTTGTGTAGCGTTAAATTTTCAATTACGTGGAGGTGCTGGTC |
| | | | | TCCGAACTTCTGTGCAGGCCTTTTCAGAAGGTGGTCTTTCCTTAAT |
| | | | | ACCGGGTTTACTACATCCTCATGGAAATGGAGTTAGGGGAAATGT |
| | | | | AAAACGTTTTTTACAACCCATAGCATCAGAGTTCACTCAATCGCCT |
| | | | | CATTGTAACACTTTTTCCTGTACTTCATCGTTTGTTTTTGGAAATGT |
| | | | | TTTATCTACAAGGAGGGACTTAGAAACAAATGATGGGCGGGAGGT |
| | | | | AAGGGTTACCAAGTATCTATTTAACCACTCTCAGGGAGAGAAAAT |
| | | | | GAAACGTACAGGTGGTAGAGGAGATGCAGCAGATATTGTAAGCCA |
| | | | | TGCGTTAACTTTAGCTCTATTTTTATCAAATATTGGTTTTGTTGTGC |
| | | | | TTCACATTTTTCGTCGTTTAAAGTGGCAGACTTTACCAGACAAGGC |
| | | | | ATTGAACCTGTCGTTACTGCCTCAAGCCACAGATGATGCTAAGGTG |
| | | | | CGTCAAGTACTTTGTGAAGTAGATGTCCAGGGTCATCTGGAAGTG |
| | | | | GCTTCTGCAAAGAAATTCGAATCACTACGTGCTTACTCACGTTCTC |
| | | | | ATAGTGAAGGTTATTTCGAGGGAAGGTTTTCAGAACAAATGGGTA |
| | | | | ATGTTAGGAATTTACATCAACTTGCAGATCAGTTGATGAGTGCTGA |
| | | | | GCCTAATTTTGGTAATGATGGTAATGTTAATGGTGAGTACAGGGCT |
| | | | | AGGTATGAAGTTTTATTTAATGAGATTTCTCGTCTGTTGTCTCCGTT |
| | | | | ATTAAATGGAAACCGTCTACTCGTGAACAATGAAGCTAAATTTCA |
| | | | | GGCTTTGTTGCGTGGAATATTTCAAAATTGCGATAATCCTGCCAAG |
| | | | | GTAATTATTGAGTTCCAGCTACAGAGAGGAAGGAAAATAGACCTA |
| | | | | GTATTATCAAAATCTGCGGAAAATGATGATACTCATCCAATTGGA |
| | | | | ATAGAGTTGAAGTATGCTAACACCGCAGAACAAGTTGAACGAAAA |
| | | | | AGGGTGGAGGCAAATCGACAGTTAAGTGAATACGAATTTTGTGGA |
| | | | | GGATGCAAGCGTATTACTGGGGGAGATGCGATGGTTTTGTTATAC |
| | | | | GCTATATTAAATGCTGTAGGACAAGAGCAGGATCTGATATTGATT |
| | | | | GGTGGGCTTCGTAGAGCATCTGGGTTTTCTAGATGA (SEQ ID |
| | | | | NO: 15) |
| cinB | wNo_01980 | amino | 696 aa | MHGNNEDRELVRALLSGGCDEFSRQFVGFLNNCPSFLHSANKPGFFP |
| | | acid | | TFFFGMFSTAHDAGILVEGERVYFRFDNYGNLKVAVLTNKENRRIVR |
| | | | | CYTVADNENSPGSRFSAEEKQQVEENLPQELQEDEDLDWEEYKIFRF |
| | | | | GEECRFIHEIDRFPQRDEPGAPIFHEINPIREQGELLDLMSELANDDTGE |
| | | | | VRTNVKRILEYVIDIHDEHEDSLVFRAESDYHGFLCGFLVNFRYRALA |
| | | | | DFYPELLIGKGYADVVLLVRGVDQTNDSVPIIIELKVGDEEGLEQAKD |
| | | | | YAKSCSVSSLPIHTSSPSAVCVALNFQLRGGAGLRTSVQAFSEGGLSLI |
| | | | | PGLLHPHGNGVRGNVKRFLQPIASEFTQSPHCNTFSCTSSFVFGNVLST |
| | | | | RRDLETNDGREVRVTKYLFNHSQGEKMKRTGGRGDAADIVSHALTL |
| | | | | ALFLSNIGFVVLHIFRRLKWQTLPDKALNLSLLPQATDDAKVRQVLCE |
| | | | | VDVQGHLEVASAKKFESLRAYSRSHSEGYFEGRFSEQMGNVRNLHQ |
| | | | | LADQLMSAEPNFGNDGNVNGEYRARYEVLFNEISRLLSPLLNGNRLL |
| | | | | VNNEAKFQALLRGIFQNCDNPAKVIIEFQLQRGRKIDLVLSKSAENDD |
| | | | | THPIGIELKYANTAEQVERKRVEANRQLSEYEFCGGCKRITGGDAMV |
| | | | | LLYAILNAVGQEQDLILIGGLRRASGFSR (SEQ ID NO: 16) |
| CinA^{wPip} | wPa_0294 | nuclo- | | ATGGAATCTGGTTTGGATCACAATTACAATAAAATACTTGATATAT |
| | | etide | | TAAAAGGTGCTATTAAAGGCGACGATAATCAAGTTAAAGCAAGAA |
| | | | | AACACCTTAGAGTAGAAAGATGGTTGAGGGCTTATATTCAATTAA |
| | | | | TTGAAGATTTTGATGAGGAAAAACTAATTTTTTTTTCTGATATATT |
| | | | | CTCTGATAATTCTTGTTGGGATGGAATAAAATTAAAGAATAAAGC |
| | | | | TGTTGGTGAAAGGCTAACTGAAGAAAAAAATAAAAATGGAAAAG |
| | | | | AAAATCCGCTTGATCTTGCAGATAGATATTACTTGGCATGTAAATA |
| | | | | TTGTCTAGAAGATAAGATTCCTGGATTATTTGAACAAGTATTTATG |
| | | | | AGATTTAAGAGAAGTGCCTTTGAAGAAGATGGATCTGATGATGAT |
| | | | | CTGAGAAGAGAATTATTGGAAAATATCGAAGAAACTAGCCCTATA |
| | | | | GAAGCTTTCTGGTCTTTTCTTATTGATAAGCAGATTGGAAAACTAA |
| | | | | ACGAATATAAATCAGTTGAAGGTTTGCAAAAATCCATACAGATAA |
| | | | | ATTCTAATAAAAACTGGGAAGAAGGTATAGAGTTCTTCTATAATA |
| | | | | AATTACACAATGATTCCAGTATTTCTAGTCAAGATAAAGATGATCT |
| | | | | GTTAATTGAAGCAGCTTTATCTGCAGTAAAGGGTTACAAAGAAGT |
| | | | | AGACACCATAGAGTTTTGCCTGTCTAAAATGGATGATGAGCAAAA |
| | | | | GAAAAAATTACTAGATAGAGATTATAAGGAAAATACTTATTATGC |
| | | | | AGTGTTGAATGTGCTAGTAGGTCAGTATTACTTTGATTCTTTTATG |
| | | | | GAATTAAGCCGATTGTGTAGTCAGATTGAATGTGAACGTTACACA |
| | | | | ACTTTTTTATCTTCATTATCAGATCAAGTACTGAAGAATCCAGATC |
| | | | | TGTCTGAAGAAACAAAAAAATGTATGATGAATGTTTGGGAACGTA |
| | | | | TAATAAAATTAAAAACTCAAGACCGCGGGGAGCAATCTATTTCCT |
| | | | | CTATTTTTGTAGACTATTCAGTTACATATACAATAGCAAATTTAAT |
| | | | | TGTGGATCCAAGTAGACAAGGGGTAAGTAAAGAAGAAATATTAG |
| | | | | GGAAGATATTAAAGCACGTAAAAGAAATGAGTGGTGAAGAGATG |
| | | | | ATAAAGGTTAAAGATTCTGTATTAAGTAAAATTCAGTTATTTCATG |
| | | | | GGGGTAAAAAATTGCAGTTAGGAGAACAAGTATTTTCTAAATTAG |
| | | | | CTCAAGAAGCTTCTAAAGAATCAATTTTGCGTGAAGCTGGTGATA |
| | | | | CTTTGCCACAGTCAAGTCTCAGTACGACTGATACCCCATATAATAT |
| | | | | AAAATCTTTAAGCCATAGCAAATAG (SEQ ID NO: 17) |
| CinA^{wPip} | wPa_0294 | amino | | MESGLDHNYNKILDILKGAIKGDDNQVKARKHLRVERWLRAYIQLIE |
| | | acid | | DFDEEKLIFFSDIFSDNSCWDGIKLKNKAVGERLTEEKNKNGKENPLD |
| | | | | LADRYYLACKYCLEDKIPGLEEQVFMRFKRSAFEEDGSDDDLRRELL |
| | | | | ENIEETSPIEAFWSFLIDKQIGKLNEYKSVEGLQKSIQINSNKNWEEGIE |
| | | | | FFYNKLHNDSSTSSQDKDDLLIEAALSAVKGYKEVDTIEFCLSKMDDE |
| | | | | QKKKLLDRDYKENTYYAVLNVLVGQYYFDSFMELSRLCSQIECERYT |
| | | | | TFLSSLSDQVLKNPDLSEETKKCMMNVWERIIKLKTQDRGEQSISSIFV |
| | | | | DYSVTYTIANLIVDPSRQGVSKEEILGKILKHVKEMSGEEMIKVKDSV |
| | | | | LSKIQLFHGGKKLQLGEQVFSKLAQEASKESILREAGDTLPQSSLSTTD |
| | | | | TPYNIKSLSHSK (SEQ ID NO: 18) |
| CinB^{wPip} | wPa_0295 | nucle- | | ATGCCAAGTAATGTCAAGCCGCTTGAGTTGGTACAGCTTCTGTTAA |
| | | otide | | TGAGAAATAAATCAAAAGACGAGTTCCTAGATTTTCAAAAAAGGT |
| | | | | TCCAATCGTTTATCAATCAATCTCCTTCTTTTTTGCATTCAGTTGGA |
| | | | | AAGCCAGGCTTTTTCCCTAGTTTCTTTTTTGGTATGTTTGCTACTGT |
| | | | | ATTAGACACAGAACTTGCTACTAAAATTGGTATTAAAAAACTTCAT |
| | | | | TTTCGTTTTGATGATAATAGAACTTTAAAAATAGCTATATTAACTA |
| | | | | ATGAGGGACTTAAGTGTATAACGATGTCTGATCAAGTTGATGGTA |
| | | | | ACATGCATCTAAAGTTCTCTCAAGGAGAGTTAGAAAAAATAGCAC |
| | | | | AGAAATGGAAAATGGGAGCAGAGTTTGATAAACTAGAAAAAGAA |
| | | | | GAGCATGAAATAACAATTACAGGAAAAGAAGTAAAGCACGGAAA |
| | | | | GGTTGATCCAGCTTTTAGTAAAAAGACTGATTATTCACAAAAAGG |
| | | | | TTTTACAGAAATAGAAAAAGATCGTGACCAACAAGACCTAGAGAG |
| | | | | CTTAATTTCAAAATTGAGTAATCAAGATTTCGAAGAAGTAAAAAA |
| | | | | GAACGCTAGAAGAATGTTTAATTATATTACAAATGTCTATAAGAA |
| | | | | ATATGAAAAAGAAACTCTATTTAGCGGTAAAGAATCAAGTCATCA |
| | | | | TGGGTTTTTAGCTGGGTTTTTGATAAATTTTAAGTATCGTTTTCACC |
| | | | | TAAAACTTTATCTCGAATTATTTGCTGGAAAAGGTTACGCAGACAT |
| | | | | TATTTTGCTTGTGCGCGGTTCTGATAAGTCGCTAAGCTCTATTCCTA |
| | | | | TTATTATTGAGCTTAAAGCAGGTACTGGTGAGATAAGTACAGTGA |
| | | | | TAAAAGCATTGAAGCAAGCACAAGATTATGTTAAGGGCTCTTTTTC |
| | | | | TAACTCTATAAGAATGATTACTATAGCTAATGAAGCTATTTGTGTA |
| | | | | GGATTAAATTTTGACATGGTTCATCACGAAAATGTTAAAATTGATG |
| | | | | TAGAAAATTTTCTTAGTCGAGAAGGTAATTCTGTAATAGAAAAGTT |
| | | | | ACTTGGCACTGAAGCAACGAATGCTGAGGTGATAAGAACACAGCT |
| | | | | AGAGTATCTTTACTATGGAATTGTTTGGAGCAATGGTGGAAGTGAT |
| | | | | AATATTAATTATGTCAGCAGAATGATCTTAGGTCAGCTAGTACTTA |
| | | | | TTTCTAATATTATTAAGCGTGAAAAGTTAGGTAAACATATTTTTAT |
| | | | | TTATGATCAAAATGATAAAATGGTTACTGGATCACAGAAACGCCC |
| | | | | AGAAGCAGCAAAAGAAAGTATTGAGGATTGTGTTACAACTATAGT |
| | | | | GCTAACTTTAGGTAAGAAGGTGCTTATACTCAACATAAATGAAAA |
| | | | | AAATGAATTTGCATTGAGAGTGCCAGATAATAAAGGAATTCCTAT |
| | | | | TGAAAATATTAGGAGAATTCAAAACGTCAATGACATAAAGATACA |
| | | | | AGAAATAACCTGTAACTTATACAGTACGCCTAGTAATAAGAATCC |
| | | | | ATTTGATCAGTACTGTAATAAGAATAAGGGAATTACAGTAAATAC |
| | | | | GTATGACTCATTGGACAAATACAAAAGAGGTAAAGAAATTTTACA |
| | | | | AGGTAATTTTACTCGAATTGTGGAAAATAAAAAATTTAAAGCAGC |
| | | | | TTTGAGCAAAGCTATAGAATCTGGTAAATATGATGATTACAAAAA |
| | | | | ACTATTTGAAGAAATTTCTCATATACTACATCCTTTCAAATCATTA |
| | | | | ATAAGCAATGAGGCTACATTTCAAGCTGTATTGCATGGTTTATTTA |
| | | | | GTAGCTACGGAGAAGATAATATAAAAGTTATTACTGAATTTCAAA |
| | | | | TAGGTGGTGGAGAGAAGTTGGATGTTATGTTGGTTATAAATGCTA |
| | | | | CTGATCAAAAAAAAGAATACCCCCCAGTTGGAATAGAGCTAAAAT |
| | | | | TTGCTAAGAAAGGAGAATTGGATAAAAAAGAAAAAGATGCTAAG |
| | | | | GACCAGTTGAAAAGATATAAAGAAGGTGAAGCGTATAAGGTAATT |
| | | | | ACTGATGCTGGCAAAGTGAAACTGATATATGCTGTTTTTAATAAAG |
| | | | | GTGCAACAGATGAAGGTTCCCTTATAAAAATTGGTAATGAGTTTGT |
| | | | | AGAGGTAGATGTAAGACATAGCTCTGTGGTTGCTTTTGGTCAACA |
| | | | | GCCAGGTAGTCTCCAACAACCTTATGTTAAACAAGCAGGTCTATCT |
| | | | | CGAGCAGTTAATCAGTGA (SEQ ID NO: 19) |
| CinB^{wPip} | wPa_0295 | amino | | MPSNVKPLELVQLLLMRNKSKDEFLDFQKRFQSFINQSPSFLHSVGKP |
| | | acid | | GFFPSFFFGMFATVLDTELATKIGIKKLHFRFDDNRTLKIAILTNEGLK |
| | | | | CITMSDQVDGNMELKFSQGELEKIAQKWKMGAEFDKLEKEEHEITIT |
| | | | | GKEVKHGKVDPAFSKKTDYSQKGFTEIEKDRDQQDLESLISKLSNQD |
| | | | | FEEVKKNARRMFNYITNVYKKYEKETLFSGKESSHHGFLAGFLINFK |
| | | | | YRFHLKLYLELFAGKGYADIILLVRGSDKSLSSIPIIIELKAGTGEISTVI |
| | | | | KALKQAQDYVKGSFSNSIRMITIANEAICVGLNFDMVHHENVKIDVE |
| | | | | NFLSREGNSVIEKLLGTEATNAEVIRTQLEYLYYGIVWSNGGSDNINY |
| | | | | VSRMILGQLVLISNIIKREKLGKHIFIYDQNDKMVTGSQKRPEAAKESI |
| | | | | EDCVTTIVLTLGKKVLILNINEKNEFALRVPDNKGIPIENIRRIQNVNDI |
| | | | | KIQEITCNLYSTPSNKNPFDQYCNKNKGITVNTYDSLDKYKRGKEILQ |
| | | | | GNFTRIVENKKFKAALSKAIESGKYDDYKKLFEEISHILHPFKSLISNE |
| | | | | ATFQAVLHGLFSSYGEDNIKVITEFQIGGGEKLDVMLVINATDQKKEY |
| | | | | PPVGIELKFAKKGELDKKEKDAKDQLKRYKEGEAYKVITDAGKVKLI |
| | | | | YAVFNKGATDEGSLIKIGNEFVEVDVRHSSVVAFGQQPGSLQQPYVK |
| | | | | QAGLSRAVNQ (SEQ ID NO: 20) |

1. A genetically modified arthropod, said arthropod comprising:
(i) a bacterial operon or two independent chromosomal insertions encoding at least two cytoplasmic incompatibility factors wherein the bacterial operon or the two independent chromosomal insertions is from Wolbachia; and
wherein the bacterial operon or the two independent chromosomal insertions encode:
the cytoplasmic incompatibility factors WD0631 and WD0632; wherein WD0631 comprises the amino acid sequence SEQ ID NO:2, or a sequence at least 60% identical to the amino acid sequence SEQ ID NO:2; and wherein WD0632 comprises the amino acid sequence SEQ ID NO:4, or a sequence at least 60% identical to the amino acid sequence SEQ ID NO:4; or
the cytoplasmic incompatibility factors CidA^{wPip} and CidB^{wPip}, wherein CidA^{wPip} comprises the amino acid sequence SEQ ID NO:6, or a sequence at least 60% identical to the amino acid sequence SEQ ID NO:6; and wherein CidB^{wPip} comprises the amino acid sequence SEQ ID NO:8, or a sequence at least 60% identical to the amino acid sequence SEQ ID NO:8; and
(ii) a promoter operably linked to the bacterial operon or the two independent chromosomal insertions;
wherein the expression of the cytoplasmic incompatibility factors in a male arthropod causes a reduction in viable offspring in comparison to a male arthropod lacking the cytoplasmic incompatibility factor.

2.-7. (canceled)

8. The arthropod of claim 1, wherein the bacterial operon or the two independent chromosomal insertions encodes the cytoplasmic incompatibility factor WD0631, wherein WD0631 comprises the amino acid sequence SEQ ID NO:2.

9. The arthropod of claim 1, wherein the bacterial operon or the two independent chromosomal insertions encodes the cytoplasmic incompatibility factor WD0632, wherein WD0632 comprises the amino acid sequence SEQ ID NO:4.

10. (canceled)

11. (canceled)

12. The arthropod of claim 1, wherein the bacterial operon or the two independent chromosomal insertions encodes the cytoplasmic incompatibility factor CidA^{wPip}, wherein CidA^{wPip} comprises the amino acid sequence SEQ ID NO:6.

13. The arthropod of claim 1, wherein the bacterial operon or the two independent chromosomal insertions encodes the cytoplasmic incompatibility factor CidB^{wPip}, wherein CidB^{wPip} comprises the amino acid sequence SEQ ID NO:8.

14. (canceled)

15. (canceled)

16. (canceled)

17. (canceled)

18. The arthropod of claim 1, wherein the reduction in viable offspring is greater than 50%.

19. The arthropod of claim 1, wherein the arthropod is an insect.

20. The arthropod of claim 19, wherein the insect is selected from the genera consisting of Aedes, Culex and Anopheles.

21. The arthropod of claim 20, wherein the insect is selected from the group consisting of Aedes albopictus, Aedes aegypti, Aedes polynesiensis, and Anopheles gambiae.

22. (canceled)

23. A method for controlling a population of target arthropods, comprising:
(i) providing a bacterial operon or two independent chromosomal insertions encoding at least two cytoplasmic incompatibility factors, wherein the bacterial operon or the two independent chromosomal insertions is from Wolbachia; and
wherein the bacterial operon or the two independent chromosomal insertions encode:
the cytoplasmic incompatibility factors WD0631 and WD0632; wherein WD0631 comprises the amino acid sequence SEQ ID NO:2, or a sequence at least 60% identical to the amino acid sequence SEQ ID NO:2; and wherein WD0632 comprises the amino acid sequence SEQ ID NO:4, or a sequence at least 60% identical to the amino acid sequence SEQ ID NO:4; or
the cytoplasmic incompatibility factors CidA^{wPip} and CidB^{wPip}; wherein CidA^{wPip} comprises the amino acid sequence SEQ ID NO:6, or a sequence at least 60% identical to the amino acid sequence SEQ ID NO:6; and wherein CidB^{wPip} comprises the amino acid sequence SEQ ID NO:8, or a sequence at least 60% identical to the amino acid sequence SEQ ID NO:8; and a promoter operably linked to the bacterial operon or two independent chromosomal insertions;
(ii) transforming a population of male arthropods with the bacterial operon or two independent chromosomal insertions; and
(iii) releasing the male arthropods amongst a population of target arthropods, wherein the release of the male arthropods reduces the population of target arthropods.

24.-29. (canceled)

30. The method of claim 23, wherein the bacterial operon or the two independent chromosomal insertions encodes the cytoplasmic incompatibility factor WD0631, wherein WD0631 comprises the amino acid sequence SEQ ID NO:2.

31. The method of claim 23, wherein the bacterial operon or the two independent chromosomal insertions encodes the cytoplasmic incompatibility factor WD0632, wherein WD0632 comprises the amino acid sequence SEQ ID NO:4.

32. (canceled)

33. (canceled)

34. The method of claim 23, wherein the bacterial operon or the two independent chromosomal insertions encodes the cytoplasmic incompatibility factor CidA^{wPip}, wherein CidA^{wPip} comprises the amino acid sequence SEQ ID NO:6.

35. The method of claim 23, wherein the bacterial operon or the two independent chromosomal insertions encodes the cytoplasmic incompatibility factor CidB^{wPip}, wherein CidB^{wPip} comprises the amino acid sequence SEQ ID NO:8.

36. (canceled)

37. (canceled)

38. (canceled)

39. (canceled)

40. The method of claim 23, wherein the arthropod is an insect.

41. The method of claim 40, wherein the insect is selected from the genera consisting of Aedes, Culex and Anopheles.

42. The method of claim 41, wherein the insect is selected from the group consisting of Aedes albopictus, Aedes aegypti, Aedes polynesiensis, and Anopheles gambiae.

43.-111. (canceled)


SEQUENCE LISTING (24 sequences)

SEQ ID NO: 1 - DNA, 1425 bp, Wolbachia pipientis
  source 1..1425: mol_type = genomic DNA
atgccaatagaaacaaaacgtcaggctgaagtgcttaaaaagctacaagatgtgataaaacatacagatcgtgacattgcggctggaagaaagttagctattaaaaggtgggtcgagacctatatagagtatatcaaactttttaaggatgataagctggaattcttatataatgtttttcgagatgaaggttgttggttaggtacaaggttaaataatactgttttaggtcagaaattgactgaagagaaaataggagaaatcgataacccactaccaaggtatggtatggcatctaggtactgtataacgggcaagataggtgattttttcaacaaacagtttgtactctctagaggtcaatttacttcagaagaggtagatagtcaaggtaatccgatcagtgatcaatatgtaagaaacattctgctatcatccatgaagagaaatggtcctgtgtttgatttctggatcgatagagaatctggggaattaaagaagtatgatgcagtagaaggttttgacagtactgtaaaacttaagtggagcgaaggggtagagtatttttataatcagttagaggaaaaagataaggagaagaagcttacagaagctattgttgctctttctcgtcctcaatctgttaagagagacgctcctattttagatttttgtgtaaggaatataggcgataaagatactcttttacagaaattattgcagaaagataagggagtatatttccttcttgctgaattaatagagtcatgtttttttgatacggttcatgatttggtacagtgctggtgttataaaggcgtttcagcaggaggagactgttcggacaagatattctcacagcaagactatgaactttttctttattcactttcaaatgtgatgttgaaaaatcctgagttaagtgttcaagctagatcccttattatggagatttggaaatgtgaacgctttgctgaatacagagagacctctgttaatacttctaattatacagttcctataaagagtgtacttgggggattaatcattaattggaaacgagaagatgtttgtaagcccgatagggaaatagagaaagaagaaatattagatatgatttcatttgccaaaggttgctttcctgaaaagtttgacctttttaaagaagtcatgatagaaaatcttagaatatgtggtagggaaggaaagaggaaaggtgtagattacggcaagtttgcagaagagttatttcttcagttagagaaagtaactttaccttctgtaggtgatggtccttggaataatttgcggtctcaatctaaggtatctttgccacttgatggttctggtgatggcccacagtctgagtttgaagctcctagtgtgagtggtatttctggttctcataagaaaagaagaatctag

SEQ ID NO: 2 - protein, 474 aa, Wolbachia pipientis
MPIETKRQAEVLKKLQDVIKHTDRDIAAGRKLAIKRWVETYIEYIKLFKDDKLEFLYNVFRDEGCWLGTRLNNTVLGQKLTEEKIGEIDNPLPRYGMASRYCITGKIGDFFNKQFVLSRGQFTSEEVDSQGNPISDQYVRNILLSSMKRNGPVFDFWIDRESGELKKYDAVEGFDSTVKLKWSEGVEYFYNQLEEKDKEKKLTEAIVALSRPQSVKRDAPILDFCVRNIGDKDTLLQKLLQKDKGVYFLLAELIESCFFDTVHDLVQCWCYKGVSAGGDCSDKIFSQQDYELFLYSLSNVMLKNPELSVQARSLIMEIWKCERFAEYRETSVNTSNYTVPIKSVLGGLIINWKREDVCKPDREIEKEEILDMISFAKGCFPEKFDLFKEVMIENLRICGREGKRKGVDYGKFAEELFLQLEKVTLPSVGDGPWNNLRSQSKVSLPLDGSGDGPQSEFEAPSVSGISGSHKKRRI

SEQ ID NO: 3 - DNA, 3501 bp, Wolbachia pipientis
  source 1..3501: mol_type = genomic DNA
gtggatggagatcttgatggttttagacaagagtttgaatcctttttagatcaatgtccatttttcttgtatcatgtaagtacaggacgtttccttcctgtattctttttcagtatgtttgctactgctcatgatgctaatatcttaaaagcaaatgagagagtgtattttcgttttgataatcatggtattgatacaggtggtagaaatagaaatacagggaacctaaaagtcgctgtttatcatgacggacagcaagttgtcagatgctacagtatttctgatcgtcttaatagtgatgggttaaggttcagtacaagggaaagaaatgctctagtgcgagagattagagggcaaaatccaaatttaagggaagaagacctaaattttgagcaatacaaagtatgcatgcatggaaagggcaagagtcagggagaggcgattgcaacagtattcgaggtgattcgtgaaaaagattctcaaggtagagatagatttgctaaatattcagcgtctgagattagccttcttaggcatatagaacgcaataggcttaatgggattaatgcgcctgcgccacgcagtttgttgacagttaaggaaataggaagtatacgactcaatcaagatcagagagtacagcttggtcatttggtcaattttgtgcaagttgcaccgggtcagcaagggattttcagttttatggaagtgctagcaagtaaccaaaaaataaatatagaacgtggaataaatgaaggaattttgccatacataactcgaatctatcgtagttacctaggcagcctacaaaatgacattcaaaatcgcagtcaaaagtttgagagtcacggatttttcttaggtttgttggcaaattttattcatctctacacaatagatattgaccttgacttgtctcctggaaattcatatgttgcttttcttatatgtcatcaggcagagagagaaaacattcctatcgttattaatgttactagatggaggacatcgtctgatattgcattaaaccgcgctagagctgatgctaaaagattacatgtttcttcatttatatctattcacactgaatcaagaaatgctgtttgtattggattaaattttaatctgaatatagatccttttagtattgatacagtagagtttttagagaatagatttcctttggtacaaagattatttgagtgtttggaggatgaaggaattagagaaaatattagagatttcttgcttcaacatcttcctaacgaaataccaagaaatgcagagaattataacagaatatttgattgcataactggttttgcttttgggaatagtattttagaagagttcagattagtaaacgcagttcaacaacgtgtaagaaagtatatatttagatatggtgatgagaatcatgctttaaccatggtcttccatactcaaggttctgatatagttatacttcatattagagataacaacgctgtacaacaaggagccatcaatttacaagatcttaatgttgacggaaataatgttcatgtacgggaagtttcatgcacacttaataatcaacttggccttaatattcatacagataaccttggtttatatcacaattaccaaaataataatgcaaataattttcttggtggtaatcttgtgcaagtgcctaatgctggaaatgtgcataatgctttaaatcaagttatgaatgatggctggcaagatagatttcagcatcaagaattatttagaaacatttctgcagtattaatgccagaagatacgcatggcaatatgataatagatgtaaatagcaaagataagtttcgctctatactacatggtacattttatgctagtgataatccttataaagtgcttgctatgtataaagttggtcaaacatatagtttaaaaaggtggcaggaagaagaaggagaaagggtaatacttacaagagttacagaacagagactaggtcttctattattaagacaacctacagcagatactcacccaattggatatgtattaggatttgctgataatgcagaagaagtagaacaggagcaagacgaggcaaggtacaaaataacagaattgatgagcaaacaaaggggatatttgcctattacttctggaaatgaggtggttttgtcttatgctgtatttaatagaggtgcacagagagcagaagactttatatctcttccacaacaagcagtgtatgtacatagacttgatcgtcgtggtcatgactcaagaccagaagtattagtgggacctgaaagtgttattgatgaaaatccaccagaaaatctattgtcagatcaaactcgtgaaaatttcaggcgcttttacatggaaaaaagaccaggacagaactcgatttttttgcttgatatagatgataatctgcacgttccatttagttacttgcaaggtactagagcacaggcaatagaaacattaaggtcaagaataaggggaggtggtacttctacagcacaaggaatattacaacaaataaacactatccttcgtagaaacaacgctcgtgaaatagaagatgtgcataatctacttgcactagactttgcaacagaaaatcaaaatttccgttattggctacaaactcatgacatgtttttcgctgcacgacaatatactttccatgatgatcgatctaatccaactaatgatcgtcatgattttgcaataacttcagtaggagtcgatggaaatcaaaatgatccaacaggtagggacttattaagtagtaacatagataactttaaacaaaaagtagattcgggtgaaaaagatagattaactgctattattaatgtaggtaatcgtcattgggttacattagttattgtccaccaaaatggaaattattatgggtattatgctgattcacttggtccagatagtcgtattgacaataatattcgaggagctttaagagaatgtgatattagcgatgataatgtccatgatgtttccgttcatcagcaaacagatggccataattgtggcatatgggcatacgaaaatgctagggatattaaccaagctattgatcaagctttacagggaaatagtaactttggagagaaaggtgaaggtattataggttatatacgtggtcttcttagtgcaggaattggaaatgacactagacaacctcaaagaaatgaacaatactttagaaatcggagaagaaatatttcacaattattccaaaatgattctctatcttctcctaggggtagattgattcaaggtcgtccaggaattcaacatgaaattgatccattactattacaatttttagaactccaatatccacagcgtggaggtgggggagcattgcaattaggcggagaaagagtgatatcaattgattttggtccgcaatctgtattggatgaaattgatggagtgaatagagtttatgatcatagcaatggtagaggcagtaggtag

SEQ ID NO: 4 - protein, 1166 aa, Wolbachia pipientis
MDGDLDGFRQEFESFLDQCPFFLYHVSTGRFLPVFFFSMFATAHDANILKANERVYFRFDNHGIDTGGRNRNTGNLKVAVYHDGQQVVRCYSISDRLNSDGLRFSTRERNALVREIRGQNPNLREEDLNFEQYKVCMHGKGKSQGEAIATVFEVIREKDSQGRDRFAKYSASEISLLRHIERNRLNGINAPAPRSLLTVKEIGSIRLNQDQRVQLGHLVNFVQVAPGQQGIFSFMEVLASNQKINIERGINEGILPYITRIYRSYLGSLQNDIQNRSQKFESHGFFLGLLANFIHLYTIDIDLDLSPGNSYVAFLICHQAERENIPIVINVTRWRTSSDIALNRARADAKRLHVSSFISIHTESRNAVCIGLNFNLNIDPFSIDTVEFLENRFPLVQRLFECLEDEGIRENIRDFLLQHLPNEIPRNAENYNRIFDCITGFAFGNSILEEFRLVNAVQQRVRKYIFRYGDENHALTMVFHTQGSDIVILHIRDNNAVQQGAINLQDLNVDGNNVHVREVSCTLNNQLGLNIHTDNLGLYHNYQNNNANNFLGGNLVQVPNAGNVHNALNQVMNDGWQDRFQHQELFRNISAVLMPEDTHGNMIIDVNSKDKFRSILHGTFYASDNPYKVLAMYKVGQTYSLKRWQEEEGERVILTRVTEQRLGLLLLRQPTADTHPIGYVLGFADNAEEVEQEQDEARYKITELMSKQRGYLPITSGNEVVLSYAVFNRGAQRAEDFISLPQQAVYVHRLDRRGHDSRPEVLVGPESVIDENPPENLLSDQTRENFRRFYMEKRPGQNSIFLLDIDDNLHVPFSYLQGTRAQAIETLRSRIRGGGTSTAQGILQQINTILRRNNAREIEDVHNLLALDFATENQNFRYWLQTHDMFFAARQYTFHDDRSNPTNDRHDFAITSVGVDGNQNDPTGRDLLSSNIDNFKQKVDSGEKDRLTAIINVGNRHWVTLVIVHQNGNYYGYYADSLGPDSRIDNNIRGALRECDISDDNVHDVSVHQQTDGHNCGIWAYENARDINQAIDQALQGNSNFGEKGEGIIGYIRGLLSAGIGNDTRQPQRNEQYFRNRRRNISQLFQNDSLSSPRGRLIQGRPGIQHEIDPLLLQFLELQYPQRGGGGALQLGGERVISIDFGPQSVLDEIDGVNRVYDHSNGRGSR

SEQ ID NO: 5 - DNA, 1476 bp, Wolbachia pipientis
  source 1..1476: mol_type = genomic DNA
atgccaacacagaaagagcttcgggatacgatgtccaaaaaattacaggaagctattaaacatccagatccagcagttgttgccgggaggaagtcagctatcaagagatgggtgggagtccttcaagataactttatggagcacataaaatactttaagggtgataagttgaagtttttgcacaatgtatttcaagatgaaggttgctggtcaggtgtaaggttggataatgctgctttaggtcaaaggtttactgaagaaaaaataggtggaatagataatccacttcgcaaatatgagatggcttgtagttactgtgtggtggataaaattcatcctctctttcaaaaaagatttgaatcttataggaacaagtttcctcctggtgcatttgatggtaaaactgaaactgaatttggcaaatacgtacgaaactcgttactagatagcataaagaggaaaggtcctgtatttgatttctggattgatagagaatctggggaattaaagaagtatgatgcagtagaaggttttgacagtgctgtaaaatttaagtggagtgaaggggtagagtatttttataatcatttaaaagaagaagataaggaaaagaagctcacagaagctattcttgctctttctcgcgttcaatctgttgagaaagacgcccctattttagatttttgtgtaaataagatagtcgataaagatactcttttacagaaattatcacagaaagataaaggagtatattccctttttgctgaattaatagagtcatgtttttttgatacggttcatgatttggtacagtgctggtgttataaagaagtttcagcaggaggagaccattcagagaaaatattctcacagcgagactatgagctttttctttcctctctttcagacacaatgttgaaaaatcctgagttaagcgttcaagctagatctcttattatggaattttgggaatgtggtagcttgtatcaatacagaaaagctgctgttaatacttctaattatacagttcctacaagtggtgtatttgcagagttaatagtcaattggagacgagaagacatttataagactgatgaagaaaaagaaatagagaaaaaagaaatattagatatgatgtcatttgccaaagattgctttcctgaaaagtttgagctctttaaaaaactaataataagagaccttagattatgcggtagggaaggtaaaagagtaaatgtagattacggtctgtttgcagaagaattattctctgagttagagaaaacaattttaccacctggtcctgtaggtgatggtccttgcagtaatttgcgatcacgatctaaagctcatggtagtaagaaaacaactttgccagttgatgatagtccgcagtctgagcttggaactcctagtgtaagtggtgtttcttcttataagaaaaaaagcgtctttacgcttagtggtaataagtaa

SEQ ID NO: 6 - protein, 491 aa, Wolbachia pipientis
MPTQKELRDTMSKKLQEAIKHPDPAVVAGRKSAIKRWVGVLQDNFMEHIKYFKGDKLKFLHNVFQDEGCWSGVRLDNAALGQRFTEEKIGGIDNPLRKYEMACSYCVVDKIHPLFQKRFESYRNKFPPGAFDGKTETEFGKYVRNSLLDSIKRKGPVFDFWIDRESGELKKYDAVEGFDSAVKFKWSEGVEYFYNHLKEEDKEKKLTEAILALSRVQSVEKDAPILDFCVNKIVDKDTLLQKLSQKDKGVYSLFAELIESCFFDTVHDLVQCWCYKEVSAGGDHSEKIFSQRDYELFLSSLSDTMLKNPELSVQARSLIMEFWECGSLYQYRKAAVNTSNYTVPTSGVFAELIVNWRREDIYKTDEEKEIEKKEILDMMSFAKDCFPEKFELFKKLIIRDLRLCGREGKRVNVDYGLFAEELFSELEKTILPPGPVGDGPCSNLRSRSKAHGSKKTTLPVDDSPQSELGTPSVSGVSSYKKKSVFTLSGNK

SEQ ID NO: 7 - DNA, 3525 bp, Wolbachia pipientis
  source 1..3525: mol_type = genomic DNA
atgagtaatggtgatggacttattaggagtttggtggatggagatcttgaaggattcagacaaggatttgaatcttttttagatcaatgtccatctttcttgtatcatgtaagtgcaggtcgtttccttcctgtattcttttttagtatgttttctactgcacatgatgctaatatcttaaatgcaaatgagagagtctattttcgttttgataaccatggtgttaatccacgtaatggtgaaaatcgaaatacggcaaacctaaaagttgctgtttatcgtgacggacagcaagttgtcagatgctacagtatttctgatcgtcctaatagtgatgggttgaggttcagtacaagggagagaaatgctctagtacaagagattagacggcaaaatccaaatttaagggaagaagacctaaattttgagcaatacaaagtatgcatgcacggaaagggcaagagtcagggagaggcaattgcaacggtattcgaggtaattcgtgaaaaagatcgtcaaggtagggataaatttgccaaatattcagcatctgaggttcatttcttgaggcaactctttagaaatcacagattaacaattaaggaaatagaaggaagacaactcaatcaaaatcagctcagacaacttggtaggtcagtcaattttacacgagtagaaccaggtcagcagaggattgacaactttatggaaatgctagcaagtaaccaaagacaagatgtaagggattctctccgaggagatattttagaatatgtaactgatacctataacaattatagggcacagatagaaaataatattgaaggtcgcagtcaaaagtttgagagtcatgggtttttattaggtttcttagcaaattttagtcatcgctacacaataggcgtcgatcttgacttatctcctagaaactcacatgttgcatttcttgtacgtcatcaagtagaaagagaaaatattcctattgttattaatcttgctacaagggcaccgccctatatcgcattaaaccgcgccagaagtcacgctgaaagattgcatgttttttcatttatacctatccatactgaatcaagaaatactgtctgtgttggattaaattttaatttaaatctagatccttttagtgttgatacagtagggcttcaacaggatagatttcctttagtacaaagattatttgagtgtttggagaatgaaggaattagagaaaatattagagatttcttgcttcaccatcttcctgctgaaataccaagaaatgcagagaattatgatagaatatttgattgcataactggttttgcttttgggaatagtgcttttgataggcaccctttagaactagaagaggaagacgaagcacctataacaaagtacatatttagacatggtgatgagggtttaagatgtttaactatggtctttcatgctgaaggttctgatatagttatacttcatattagagctcacgatgcgcaacaacaaggagccatcaatttacagactcttaatgttaatggaaatgatgttcatgtgtgggaagtttcatgcacacttaataatcaacttgaactagatattgatctaccaaatgaccttggtttatatcacgattaccaaaataataatgcaaataattttcttgctggtgatcttgtacaagtgcccaatactgaaaatgtacataatactttaaatcaagttgtgaatgatggctggaaaaatatagctcagcatagaggattatttcaagagatctctggagcattgatgccgcttgtggatacaataaatgttaatagtgaggataagttccgttctatactacatggtacattttatgctagtgataatccttataaagtgcttgctatgtataaagttggtcaaacatatagtttaaaaagggggcaggaagaagaaggagaaagggtaatactcacaagaattacagaacagagattagatcttttattattaagacaacctagagagaatgacctagatactcacccaattggatatgtgttaagacttgctaataatgcagaagaagtaggacaacagcaaaatgatgcgagacaagaaatcggaagacttaagaaacaacacagaggatttatacctattacttctggaaatgaggtggttttgtttcctattgtgtttaatagagatgcacacgaagcaggtaatcttatacttttcccagaagggataggaagagaagagcatgtacacaggcttgatcgtcatgttcgcagctcaagaccaggaggattagtgggacctgaaagtgttattgatgaaaatccaccagaaggtctattatcagatcagactcgtgaaaactttaggcgtttttacgaagaaaaagcaccaggacaaaattcgatttttttgcttgatataggcgacaatctacatgttccctttagttacttgcaaggtactagagcacaggtaatagaaacattaaagtcaagaataaggggaggtggtactcctacagcacaaggaatattacaacaaataaatgctatccttcgtagaaacaacgctcgtgagatagaagatgtgcatgatctacttgcactagactttgcaacagataatcaaaattatcgttattggctacaaactcatgacatgtttttcgctgcacgacaatatactttccttgataatcaatctcattcaactaatgatcattatggttttgaaataacttcagtaggagtcgatggaaatcaaaatgatccaacaggtaggggcttattaagtagtcacataactaactttaaacaaaaagtagattcgggtgaaaaagatagattaattgctattattaatgtaggtaatcgtcattgggttacattagttattgtacaccaaaatggaaattattatgggtattatgctgattcacttggtccagatagtggtattgacaataatattcgaggagctttaagagaatgtgatattaacgatgataatgtccataatatttccgttcatcagcaaacagatggccataattgtggcatatgggtatacgaaaatgctagggatattaaccaagctattgatcaagctttacagggaaataataactttggagagaaaggtgaaggtattataggttatatacgtggtcttcttagtgcaggcattggaaatgacactagacaacctcgaagaaatgaacaatactttgaagatcggagaagagatatttcacaattactccaaaatgatcctaacttaccttctcgccggagtgatttaattcaagctcatccaggaattcaacatgaaattgatccattactattacaatttttaggactccaatacccacagcgtggaggtggaggagcattacaattaggcggagaaagagtgatatcaattgattttggtaacccgcagtctgcattagataaaattgatggagtgagtagagtttataaccatagcaatagtagaggtagtaggtag

SEQ ID NO: 8 - protein, 1174 aa, Wolbachia pipientis
MSNGDGLIRSLVDGDLEGFRQGFESFLDQCPSFLYHVSAGRFLPVFFFSMFSTAHDANILNANERVYFRFDNHGVNPRNGENRNTANLKVAVYRDGQQVVRCYSISDRPNSDGLRFSTRERNALVQEIRRQNPNLREEDLNFEQYKVCMHGKGKSQGEAIATVFEVIREKDRQGRDKFAKYSASEVHFLRQLFRNHRLTIKEIEGRQLNQNQLRQLGRSVNFTRVEPGQQRIDNFMEMLASNQRQDVRDSLRGDILEYVTDTYNNYRAQIENNIEGRSQKFESHGFLLGFLANFSHRYTIGVDLDLSPRNSHVAFLVRHQVERENIPIVINLATRAPPYIALNRARSHAERLHVFSFIPIHTESRNTVCVGLNFNLNLDPFSVDTVGLQQDRFPLVQRLFECLENEGIRENIRDFLLHHLPAEIPRNAENYDRIFDCITGFAFGNSAFDRHPLELEEEDEAPITKYIFRHGDEGLRCLTMVFHAEGSDIVILHIRAHDAQQQGAINLQTLNVNGNDVHVWEVSCTLNNQLELDIDLPNDLGLYHDYQNNNANNFLAGDLVQVPNTENVHNTLNQVVNDGWKNIAQHRGLFQEISGALMPLVDTINVNSEDKFRSILHGTFYASDNPYKVLAMYKVGQTYSLKRGQEEEGERVILTRITEQRLDLLLLRQPRENDLDTHPIGYVLRLANNAEEVGQQQNDARQEIGRLKKQHRGFIPITSGNEVVLFPIVFNRDAHEAGNLILFPEGIGREEHVHRLDRHVRSSRPGGLVGPESVIDENPPEGLLSDQTRENFRRFYEEKAPGQNSIFLLDIGDNLHVPFSYLQGTRAQVIETLKSRIRGGGTPTAQGILQQINAILRRNNAREIEDVHDLLALDFATDNQNYRYWLQTHDMFFAARQYTFLDNQSHSTNDHYGFEITSVGVDGNQNDPTGRGLLSSHITNFKQKVDSGEKDRLIAIINVGNRHWVTLVIVHQNGNYYGYYADSLGPDSGIDNNIRGALRECDINDDNVHNISVHQQTDGHNCGIWVYENARDINQAIDQALQGNNNFGEKGEGIIGYIRGLLSAGIGNDTRQPRRNEQYFEDRRRDISQLLQNDPNLPSRRSDLIQAHPGIQHEIDPLLLQFLGLQYPQRGGGGALQLGGERVISIDFGNPQSALDKIDGVSRVYNHSNSRGSR

SEQ ID NO: 9 - DNA, 1371 bp, Wolbachia pipientis
  source 1..1371: mol_type = genomic DNA
atgccaaaaaagatggagcgtcatgctgcagtgcttagtaagttaaagagtgttattcaacatacagattccaaggtcatggctgaaaggcgttcagctattgaaagatgggtaaaaacttacattaggcaggtagaatatcttaaagatgataagctacaattcttatacaacatatttcgcgatgaaagttgttggtcaggtacgagattgaacaatacaatcttaggacagaggtttactgaagaaaaaataggcgaaataaagaaccctcttcctatatatgatatggcatgtcgatactgcgtgatagataaaattcctttgctctttcagaagcagtttgaatcttacaaaagtagcttctcttctgaagagatagatgatgatggtaagcctgcaactagcaataacaaatatgtaaagagtgagttgttgggttatatgaagagtcaagaccctgtatttagcttttgggttgataaaaaatctggagaatttaagaagcatgtcagcgcaacagaaggatttaagaaagctatagaacttaagtggagcgaaggagtagaatatttttatagccttctaaatgaaaaagaaagagaaagagaaaggaaaattactgatgcagttactatattatcctctgttcaatgtgaccataatggtgctgttactttagacttttgtcttagtaaaatgagcgatcaagcaaaaaacaagctgtttaaagattctgagctatcaaaaaaagataaaggagtgtactctctctttagcgcgttgatacatcaaggtttttttgatacgatgcaagctatacttccgatgtttaaagataaaatactggaggataagatactttcacctaggagttatactcttcttctctcctcactttcggacatgatgctcgaaaattctgagtcaactattcaagctagggaagctataatgaaccttataaagtgtggtaatttcaataatcatgaggggcgtgaggaaaaagctgcggtatttttttctaatggaagggttccgattaagcgtgcgcttgcaggattgattgtcgattggcaacttggttgtacaaaaaaggaagaggtgttaaaggtactacagtttgccaaagagttttgtgcagttgaaagttttatgtattttaaaaaatctgttgttgataacctaaaaatggttggtagggatggtatgagaaaaaatatagactatggtaaattagcagaaaagttgtttgctgaattagatacggtatccgtgcctaacggaagaggtgattttggtggagctggtgacccacagtctacactaggaagcactgaagttagtagtttttctggtcgcaataagtag

SEQ ID NO: 10 - protein, 456 aa, Wolbachia pipientis
MPKKMERHAAVLSKLKSVIQHTDSKVMAERRSAIERWVKTYIRQVEYLKDDKLQFLYNIFRDESCWSGTRLNNTILGQRFTEEKIGEIKNPLPIYDMACRYCVIDKIPLLFQKQFESYKSSFSSEEIDDDGKPATSNNKYVKSELLGYMKSQDPVFSFWVDKKSGEFKKHVSATEGFKKAIELKWSEGVEYFYSLLNEKERERERKITDAVTILSSVQCDHNGAVTLDFCLSKMSDQAKNKLFKDSELSKKDKGVYSLFSALIHQGFFDTMQAILPMFKDKILEDKILSPRSYTLLLSSLSDMMLENSESTIQAREAIMNLIKCGNFNNHEGREEKAAVFFSNGRVPIKRALAGLIVDWQLGCTKKEEVLKVLQFAKEFCAVESFMYFKKSVVDNLKMVGRDGMRKNIDYGKLAEKLFAELDTVSVPNGRGDFGGAGDPQSTLGSTEVSSFSGRNK

SEQ ID NO: 11 - DNA, 2265 bp, Wolbachia pipientis
  source 1..2265: mol_type = genomic DNA
atgcatgggttagttagaagtttaataaatggaaattgtggagaattcacggaaaagtttgaatatttcttggattcatgtccatcttttctgcattcagttggcaaagatcacttttttcctgcgttcttttttggcatgtttgctactgcacatgattctggtgttgcaaacaatgatgaaagaatcttctttcgttttgataatgatccaggtagtcctggaaggggaaatctaaaggttgcaattctaacaactgatggaaataacagaagagttgtaaggtgctatactattgctgacagagagaatagctacggttctaggtttagccagcaggaaagggagcagctggaaggtatcctgcgagatgaagagcttgaatggcaagagtataaaacatttatatgggcggataatcaaggtgaagatgaagaagaggaagcagtaagatgtaggatatttcaggcaggacaagggccgtttactggaaatcatgcatcttatttaactcgtagacatagttttcaagagattaccagaacacctgggctgcaaaataattatttaccggatttgatgaatcagctagaaagtgatgatgcagatgatgtacacgacactactgaggaagtgtttcagcatattattggtgtctacgatagatatagtcaggcattggacttctatggtagagagtctgactatcatggttttgtttccggtgttttgatgcattttagatatcgcaatgtagccaatatttaccttgagctgtttgtaggtggtggatatgcagatattacttctattgtacgtggtacacagaggttaattaattctgttccctgtgtaactgaacttaaggcaggcagaagagcagataggaatgctggccgtgcattagagcaggctggaaattatgttaatggatgtcccgtttcatccatatctattccaacattatcaccaagagctgtctccgctggagtgaatttcgattttggtaacccaggacgtttacagcttggtgtgagggcttttttagcaaaaggttcttctttaatggaaagattatttgaacctgtagaggatgaggagattggagaaaatgttagggattatctactccatccagcctttggtgtacctgctgtaccaggtattaggaataggggtggtgttaacgctagagatagaagaatatttctctatacaagtggatttgctttcgcaagtattgcatttgcaaaaggaactgtgccaatagaaggaaatcgtgcaatagtagataagcacttgtttcactatgacggtaatgcaaaaatgttagatgagcaaagatacaatacacaagtaaatattggagatcgtgctttgactatggttttgcatgtatcacgaggtagagaccagaaggaggaggtgatcgtatttcatgttcgccacgtattggctaatcaactttttccggacaatggattggatctatcgcgttggccgaatgctatggtacatgaagtggtgtgtaatttgaccataaatagaaggacaagaggagtaaatgataatcttggtttaactgttaatgtagaaacatttgactcgcctgctgactacctgcttgatagaggtaatcagccttttcaaggtgagcttttacgaataggtggcgttagtaatgtgcatcgcgctgcaaatgtaatgatgaatactggctgggaaaatgaagatccagacagtcatgaacggttttaccaagcaatttccaacgtgctaaatccaccccagccaaataatgcaggactccaatcattagcatgggtagtgaacagagataatgctagagaagctgggtttcatgctgcattgcatggattattttacacttgcgataatcctgctagggtagttagtgaatttcaggttggaggaggaggaaagttagacttagtattgtcacgagctataggaaggatgggaggtacttatccgattggaacagagctaaagtttgctgccactgaagcagacgtacaaaatagagaagaagaagcagatgaacaggtggagggttatctgcagagtagagggtttgatcgcattactgatggagataaaatggttttctcgtatgccgtatttaatgatcaagcgccagcaccagcacaaaatgtcccaaatacccttatagcagttagtaatgttctacgcataaaagataacttaggaattgacactgtggacgactttccttatagataa

SEQ ID NO: 12 - protein, 754 aa, Wolbachia pipientis
MHGLVRSLINGNCGEFTEKFEYFLDSCPSFLHSVGKDHFFPAFFFGMFATAHDSGVANNDERIFFRFDNDPGSPGRGNLKVAILTTDGNNRRVVRCYTIADRENSYGSRFSQQEREQLEGILRDEELEWQEYKTFIWADNQGEDEEEEAVRCRIFQAGQGPFTGNHASYLTRRHSFQEITRTPGLQNNYLPDLMNQLESDDADDVHDTTEEVFQHIIGVYDRYSQALDFYGRESDYHGFVSGVLMHFRYRNVANIYLELFVGGGYADITSIVRGTQRLINSVPCVTELKAGRRADRNAGRALEQAGNYVNGCPVSSISIPTLSPRAVSAGVNFDFGNPGRLQLGVRAFLAKGSSLMERLFEPVEDEEIGENVRDYLLHPAFGVPAVPGIRNRGGVNARDRRIFLYTSGFAFASIAFAKGTVPIEGNRAIVDKHLFHYDGNAKMLDEQRYNTQVNIGDRALTMVLHVSRGRDQKEEVIVFHVRHVLANQLFPDNGLDLSRWPNAMVHEVVCNLTINRRTRGVNDNLGLTVNVETFDSPADYLLDRGNQPFQGELLRIGGVSNVHRAANVMMNTGWENEDPDSHERFYQAISNVLNPPQPNNAGLQSLAWVVNRDNAREAGFHAALHGLFYTCDNPARVVSEFQVGGGGKLDLVLSRAIGRMGGTYPIGTELKFAATEADVQNREEEADEQVEGYLQSRGFDRITDGDKMVFSYAVFNDQAPAPAQNVPNTLIAVSNVLRIKDNLGIDTVDDFPYR

SEQ ID NO: 13 - DNA, 1473 bp, Wolbachia pipientis
  source 1..1473: mol_type = genomic DNA
atgccaaaaagtaaaactaaacgtggaacggaagatttgaagggtaatgcaggcccaagcaaaagatctcgtctcagttctgatcctaaaaaaaataaagagattatctctagcaaagtaataagtaagctgaaggatgttgttaaaggtgatagaacttcagctattgaggaatgggtcaaggctcaccctgtcacagtagagggtctaatcgttgagcaatcggacctcttatgtaatgcgtttcgtgatgaatcttgttggtcaggtgcgacactagatgttgctaaattggtaggagaattagctaaatcaggtgtgttgaatccatttgctatatataaaatagcatgtattgagtgtgtagagagtgaaattaagcaattatttgacaaggcgttagagtcttttagatctgacttatctcataaaggtgcatgtgaggaagataggaatttagcttgcagtgataagcttgcaagagttgaattgttaagttccatgggaagacgtgatcctgttttcaatttctggattgatcaagaatcaggtaaccttagagaaaatatagaagcagaagatggatttaataaggctgtagatttcaagtggagtaagggagtggaacacttctataatcgtctgtgttctgaagaaaaattagtgaaagaagagagagaaaaattgctagtttctgctattgcaaaattatctccattgcaatctagctataaacttgcttctaccttaaattcccttctaggtaaagtcataagcgcaaaagtagatcataagtcactacttgggctaccgaataagagagataggggtgtgatctatcgtcctcttagttacttagtagagcacggttttctttgcacaactaagtatgttatccagtacttgagcgagggatgttcaagatctgaagtagagaaaatgctttcacctagaggatatgcacatcttctctcatcgctttcatttgttgtagtttctaaagattatgacttggataacaggaatgaagcaaggtcagctattagcagtctttgggaatctagtgtatttaaccaaaataaaataaatgttgtcgatccttttaaagataggattgcttttgttgcaatggaaaatgcaatttcaaatttgattgtagatcaggagaacagtaaggatactcaaagtgctggcgatggtgaaaaagttgatttggtcttgagtattttaaagtttgctaaagattgttgttcagacaaaagctttaaatcattaaaagcgaggatagcaaatagtttagataaaacaaggaattctaagatgatagatgcaactagctcctgcaatttaatagaagagttgtgtaagtcagcgagaaatttgaatttattctctgctagcactgaaggtcctcaatctacgttagtgggtactaatgttagtatttcgcctgctgcagttgttaacaaatag

SEQ ID NO: 14 - protein, 490 aa, Wolbachia pipientis
MPKSKTKRGTEDLKGNAGPSKRSRLSSDPKKNKEIISSKVISKLKDVVKGDRTSAIEEWVKAHPVTVEGLIVEQSDLLCNAFRDESCWSGATLDVAKLVGELAKSGVLNPFAIYKIACIECVESEIKQLFDKALESFRSDLSHKGACEEDRNLACSDKLARVELLSSMGRRDPVFNFWIDQESGNLRENIEAEDGFNKAVDFKWSKGVEHFYNRLCSEEKLVKEEREKLLVSAIAKLSPLQSSYKLASTLNSLLGKVISAKVDHKSLLGLPNKRDRGVIYRPLSYLVEHGFLCTTKYVIQYLSEGCSRSEVEKMLSPRGYAHLLSSLSFVVVSKDYDLDNRNEARSAISSLWESSVFNQNKINVVDPFKDRIAFVAMENAISNLIVDQENSKDTQSAGDGEKVDLVLSILKFAKDCCSDKSFKSLKARIANSLDKTRNSKMIDATSSCNLIEELCKSARNLNLFSASTEGPQSTLVGTNVSISPAAVVNK

SEQ ID NO: 15 - DNA, 2091 bp, Wolbachia pipientis
  source 1..2091: mol_type = genomic DNA
atgcatggtaataatgaagatcgtgaattagttagggctttattaagtggaggttgtgatgagtttagtagacaatttgtaggttttttaaacaactgtccatcttttttgcattcggctaataagcctggcttttttcctacattcttttttggtatgttttctactgcacatgatgcaggtatattagttgaaggtgaaagagtctattttcgttttgacaattatggaaatctaaaagttgctgttctcactaataaagaaaatagaagaatagtcaggtgttatactgttgctgataatgagaacagccctgggtcaaggtttagtgcagaagagaagcagcaggtagaagagaatcttccacaagaattacaggaagatgaggatctggattgggaagagtataaaatatttcggtttggagaagaatgtaggtttattcatgaaatagatagatttcctcaacgtgatgaacctggagctccaatttttcatgaaattaacccaatcagagaacaaggtgaattgttagacctgatgagtgagttggcaaatgacgatacaggagaagtgcgtactaatgttaaaagaattttggaatatgttattgatatccatgatgaacatgaagatagcttagtgtttcgtgcagagtctgactaccacggttttctgtgtgggtttttagtaaattttagataccgagctttggctgatttctacccagagctacttataggaaaaggttatgcagatgttgttttgcttgttcgtggtgttgatcagacaaatgattcggttccaattataattgagttgaaggttggtgatgaggaaggattagagcaagctaaagattatgctaaaagttgttctgtttcgtctttgcctattcatacctcatcaccaagtgctgtttgtgtagcgttaaattttcaattacgtggaggtgctggtctccgaacttctgtgcaggccttttcagaaggtggtctttccttaataccgggtttactacatcctcatggaaatggagttaggggaaatgtaaaacgttttttacaacccatagcatcagagttcactcaatcgcctcattgtaacactttttcctgtacttcatcgtttgtttttggaaatgttttatctacaaggagggacttagaaacaaatgatgggcgggaggtaagggttaccaagtatctatttaaccactctcagggagagaaaatgaaacgtacaggtggtagaggagatgcagcagatattgtaagccatgcgttaactttagctctatttttatcaaatattggttttgttgtgcttcacatttttcgtcgtttaaagtggcagactttaccagacaaggcattgaacctgtcgttactgcctcaagccacagatgatgctaaggtgcgtcaagtactttgtgaagtagatgtccagggtcatctggaagtggcttctgcaaagaaattcgaatcactacgtgcttactcacgttctcatagtgaaggttatttcgagggaaggttttcagaacaaatgggtaatgttaggaatttacatcaacttgcagatcagttgatgagtgctgagcctaattttggtaatgatggtaatgttaatggtgagtacagggctaggtatgaagttttatttaatgagatttctcgtctgttgtctccgttattaaatggaaaccgtctactcgtgaacaatgaagctaaatttcaggctttgttgcgtggaatatttcaaaattgcgataatcctgccaaggtaattattgagttccagctacagagaggaaggaaaatagacctagtattatcaaaatctgcggaaaatgatgatactcatccaattggaatagagttgaagtatgctaacaccgcagaacaagttgaacgaaaaagggtggaggcaaatcgacagttaagtgaatacgaattttgtggaggatgcaagcgtattactgggggagatgcgatggttttgttatacgctatattaaatgctgtaggacaagagcaggatctgatattgattggtgggcttcgtagagcatctgggttttctagatga

SEQ ID NO: 16 - protein, 696 aa, Wolbachia pipientis
MHGNNEDRELVRALLSGGCDEFSRQFVGFLNNCPSFLHSANKPGFFPTFFFGMFSTAHDAGILVEGERVYFRFDNYGNLKVAVLTNKENRRIVRCYTVADNENSPGSRFSAEEKQQVEENLPQELQEDEDLDWEEYKIFRFGEECRFIHEIDRFPQRDEPGAPIFHEINPIREQGELLDLMSELANDDTGEVRTNVKRILEYVIDIHDEHEDSLVFRAESDYHGFLCGFLVNFRYRALADFYPELLIGKGYADVVLLVRGVDQTNDSVPIIIELKVGDEEGLEQAKDYAKSCSVSSLPIHTSSPSAVCVALNFQLRGGAGLRTSVQAFSEGGLSLIPGLLHPHGNGVRGNVKRFLQPIASEFTQSPHCNTFSCTSSFVFGNVLSTRRDLETNDGREVRVTKYLFNHSQGEKMKRTGGRGDAADIVSHALTLALFLSNIGFVVLHIFRRLKWQTLPDKALNLSLLPQATDDAKVRQVLCEVDVQGHLEVASAKKFESLRAYSRSHSEGYFEGRFSEQMGNVRNLHQLADQLMSAEPNFGNDGNVNGEYRARYEVLFNEISRLLSPLLNGNRLLVNNEAKFQALLRGIFQNCDNPAKVIIEFQLQRGRKIDLVLSKSAENDDTHPIGIELKYANTAEQVERKRVEANRQLSEYEFCGGCKRITGGDAMVLLYAILNAVGQEQDLILIGGLRRASGFSR

SEQ ID NO: 17 - DNA, 1338 bp, Wolbachia pipientis
  source 1..1338: mol_type = genomic DNA
atggaatctggtttggatcacaattacaataaaatacttgatatattaaaaggtgctattaaaggcgacgataatcaagttaaagcaagaaaacaccttagagtagaaagatggttgagggcttatattcaattaattgaagattttgatgaggaaaaactaatttttttttctgatatattctctgataattcttgttgggatggaataaaattaaagaataaagctgttggtgaaaggctaactgaagaaaaaaataaaaatggaaaagaaaatccgcttgatcttgcagatagatattacttggcatgtaaatattgtctagaagataagattcctggattatttgaacaagtatttatgagatttaagagaagtgcctttgaagaagatggatctgatgatgatctgagaagagaattattggaaaatatcgaagaaactagccctatagaagctttctggtcttttcttattgataagcagattggaaaactaaacgaatataaatcagttgaaggtttgcaaaaatccatacagataaattctaataaaaactgggaagaaggtatagagttcttctataataaattacacaatgattccagtatttctagtcaagataaagatgatctgttaattgaagcagctttatctgcagtaaagggttacaaagaagtagacaccatagagttttgcctgtctaaaatggatgatgagcaaaagaaaaaattactagatagagattataaggaaaatacttattatgcagtgttgaatgtgctagtaggtcagtattactttgattcttttatggaattaagccgattgtgtagtcagattgaatgtgaacgttacacaacttttttatcttcattatcagatcaagtactgaagaatccagatctgtctgaagaaacaaaaaaatgtatgatgaatgtttgggaacgtataataaaattaaaaactcaagaccgcggggagcaatctatttcctctatttttgtagactattcagttacatatacaatagcaaatttaattgtggatccaagtagacaaggggtaagtaaagaagaaatattagggaagatattaaagcacgtaaaagaaatgagtggtgaagagatgataaaggttaaagattctgtattaagtaaaattcagttatttcatgggggtaaaaaattgcagttaggagaacaagtattttctaaattagctcaagaagcttctaaagaatcaattttgcgtgaagctggtgatactttgccacagtcaagtctcagtacgactgataccccatataatataaaatctttaagccatagcaaatag

SEQ ID NO: 18 - protein, 445 aa, Wolbachia pipientis
MESGLDHNYNKILDILKGAIKGDDNQVKARKHLRVERWLRAYIQLIEDFDEEKLIFFSDIFSDNSCWDGIKLKNKAVGERLTEEKNKNGKENPLDLADRYYLACKYCLEDKIPGLFEQVFMRFKRSAFEEDGSDDDLRRELLENIEETSPIEAFWSFLIDKQIGKLNEYKSVEGLQKSIQINSNKNWEEGIEFFYNKLHNDSSISSQDKDDLLIEAALSAVKGYKEVDTIEFCLSKMDDEQKKKLLDRDYKENTYYAVLNVLVGQYYFDSFMELSRLCSQIECERYTTFLSSLSDQVLKNPDLSEETKKCMMNVWERIIKLKTQDRGEQSISSIFVDYSVTYTIANLIVDPSRQGVSKEEILGKILKHVKEMSGEEMIKVKDSVLSKIQLFHGGKKLQLGEQVFSKLAQEASKESILREAGDTLPQSSLSTTDTPYNIKSLSHSK

SEQ ID NO: 19 - DNA, 2199 bp, Wolbachia pipientis
  source 1..2199: mol_type = genomic DNA
atgccaagtaatgtcaagccgcttgagttggtacagcttctgttaatgagaaataaatcaaaagacgagttcctagattttcaaaaaaggttccaatcgtttatcaatcaatctccttcttttttgcattcagttggaaagccaggctttttccctagtttcttttttggtatgtttgctactgtattagacacagaacttgctactaaaattggtattaaaaaacttcattttcgttttgatgataatagaactttaaaaatagctatattaactaatgagggacttaagtgtataacgatgtctgatcaagttgatggtaacatgcatctaaagttctctcaaggagagttagaaaaaatagcacagaaatggaaaatgggagcagagtttgataaactagaaaaagaagagcatgaaataacaattacaggaaaagaagtaaagcacggaaaggttgatccagcttttagtaaaaagactgattattcacaaaaaggttttacagaaatagaaaaagatcgtgaccaacaagacctagagagcttaatttcaaaattgagtaatcaagatttcgaagaagtaaaaaagaacgctagaagaatgtttaattatattacaaatgtctataagaaatatgaaaaagaaactctatttagcggtaaagaatcaagtcatcatgggtttttagctgggtttttgataaattttaagtatcgttttcacctaaaactttatctcgaattatttgctggaaaaggttacgcagacattattttgcttgtgcgcggttctgataagtcgctaagctctattcctattattattgagcttaaagcaggtactggtgagataagtacagtgataaaagcattgaagcaagcacaagattatgttaagggctctttttctaactctataagaatgattactatagctaatgaagctatttgtgtaggattaaattttgacatggttcatcacgaaaatgttaaaattgatgtagaaaattttcttagtcgagaaggtaattctgtaatagaaaagttacttggcactgaagcaacgaatgctgaggtgataagaacacagctagagtatctttactatggaattgtttggagcaatggtggaagtgataatattaattatgtcagcagaatgatcttaggtcagctagtacttatttctaatattattaagcgtgaaaagttaggtaaacatatttttatttatgatcaaaatgataaaatggttactggatcacagaaacgcccagaagcagcaaaagaaagtattgaggattgtgttacaactatagtgctaactttaggtaagaaggtgcttatactcaacataaatgaaaaaaatgaatttgcattgagagtgccagataataaaggaattcctattgaaaatattaggagaattcaaaacgtcaatgacataaagatacaagaaataacctgtaacttatacagtacgcctagtaataagaatccatttgatcagtactgtaataagaataagggaattacagtaaatacgtatgactcattggacaaatacaaaagaggtaaagaaattttacaaggtaattttactcgaattgtggaaaataaaaaatttaaagcagctttgagcaaagctatagaatctggtaaatatgatgattacaaaaaactatttgaagaaatttctcatatactacatcctttcaaatcattaataagcaatgaggctacatttcaagctgtattgcatggtttatttagtagctacggagaagataatataaaagttattactgaatttcaaataggtggtggagagaagttggatgttatgttggttataaatgctactgatcaaaaaaaagaataccccccagttggaatagagctaaaatttgctaagaaaggagaattggataaaaaagaaaaagatgctaaggaccagttgaaaagatataaagaaggtgaagcgtataaggtaattactgatgctggcaaagtgaaactgatatatgctgtttttaataaaggtgcaacagatgaaggttcccttataaaaattggtaatgagtttgtagaggtagatgtaagacatagctctgtggttgcttttggtcaacagccaggtagtctccaacaaccttatgttaaacaagcaggtctatctcgagcagttaatcagtga

SEQ ID NO: 20 - protein, 732 aa, Wolbachia pipientis
MPSNVKPLELVQLLLMRNKSKDEFLDFQKRFQSFINQSPSFLHSVGKPGFFPSFFFGMFATVLDTELATKIGIKKLHFRFDDNRTLKIAILTNEGLKCITMSDQVDGNMHLKFSQGELEKIAQKWKMGAEFDKLEKEEHEITITGKEVKHGKVDPAFSKKTDYSQKGFTEIEKDRDQQDLESLISKLSNQDFEEVKKNARRMFNYITNVYKKYEKETLFSGKESSHHGFLAGFLINFKYRFHLKLYLELFAGKGYADIILLVRGSDKSLSSIPIIIELKAGTGEISTVIKALKQAQDYVKGSFSNSIRMITIANEAICVGLNFDMVHHENVKIDVENFLSREGNSVIEKLLGTEATNAEVIRTQLEYLYYGIVWSNGGSDNINYVSRMILGQLVLISNIIKREKLGKHIFIYDQNDKMVTGSQKRPEAAKESIEDCVTTIVLTLGKKVLILNINEKNEFALRVPDNKGIPIENIRRIQNVNDIKIQEITCNLYSTPSNKNPFDQYCNKNKGITVNTYDSLDKYKRGKEILQGNFTRIVENKKFKAALSKAIESGKYDDYKKLFEEISHILHPFKSLISNEATFQAVLHGLFSSYGEDNIKVITEFQIGGGEKLDVMLVINATDQKKEYPPVGIELKFAKKGELDKKEKDAKDQLKRYKEGEAYKVITDAGKVKLIYAVFNKGATDEGSLIKIGNEFVEVDVRHSSVVAFGQQPGSLQQPYVKQAGLSRAVNQ

SEQ ID NO: 21 - DNA, 22 bp, synthetic construct
  source 1..22: mol_type = other DNA
gggcgtgcccttgagttctctc

SEQ ID NO: 22 - DNA, 21 bp, synthetic construct
  source 1..21: mol_type = other DNA
cgaggatcgcataccgcactg

SEQ ID NO: 23 - DNA, 22 bp, synthetic construct
  source 1..22: mol_type = other DNA
gggcgtgcccttgagttctctc

SEQ ID NO: 24 - DNA, 20 bp, synthetic construct
  source 1..20: mol_type = other DNA
aacgctttgctttctcgctg